SEQUENCE LISTING (2 sequences)

SEQ ID NO: 1 - protein, 44 aa, Artificial Sequence
  Synthetic or recombinant peptide
HGEGTFTSDLSKQMEEEAVRLFIEWLKNGGPSSGAPPSKKKKKK

SEQ ID NO: 2 - protein, 39 aa, Artificial Sequence
  misc_feature (39)..(39): Residue modified by AMIDATION
HGEGTFTSDLSKQMEEEAVRLFIEWLKNGGPSSGAPPPS

(19) United States
(12) Patent Application Publication
(10) Pub. No.: US 2013/0040878 A1
(43) Pub. Date: Feb. 14, 2013

(54) PHARMACEUTICAL COMBINATION FOR USE IN THE TREATMENT OF DIABETES TYPE 2 PATIENTS

(71) Applicants: Louise SILVESTRE, Paris (FR); Gabor BOKA, Paris (FR); Patrick MIOSSEC, Paris (FR)

(72) Inventors: Louise SILVESTRE, Paris (FR); Gabor BOKA, Paris (FR); Patrick MIOSSEC, Paris (FR)

(73) Assignee: SANOFI-AVENTIS DEUTSCHLAND GMBH, Frankfurt am Main (DE)

(21) Appl. No.: 13/468,422

(22) Filed: May 10, 2012

(30) Foreign Application Priority Data
May 13, 2011 (EP) EP 11 166 052.8

(51) Int. Cl. A61K 38/26; A61P 3/10

(52) U.S. Cl. 514/7.2

(57) ABSTRACT

The present invention refers to a pharmaceutical combination for use in the treatment of diabetes type 2 patients.

Subject of the present invention is a pharmaceutical combination for use in the treatment of a diabetes type 2 patient, said combination comprising (a) desPro³⁶Exendin-4(1-39)-Lys₆-NH₂ (AVE0010, lixisenatide) or/and a pharmaceutically acceptable salt thereof, and (b) metformin or/and a pharmaceutically acceptable salt thereof, wherein the compound (a) is administered once daily before an evening meal. Yet another aspect is a method for treatment of diabetes type 2 patients, said method comprising administering desPro³⁶Exendin-4(1-39)-Lys₆-NH₂ or/and a pharmaceutically acceptable salt thereof, in combination with metformin to a subject in need thereof, wherein compound (a) is administered once daily before an evening meal.

In a healthy person the release of insulin by the pancreas is strictly coupled to the concentration of blood glucose. An increased level of blood glucose, as appears after meals, is rapidly counterbalanced by a respective increase in insulin secretion. In fasting condition the plasma insulin level drops to a basal value which is sufficient to ensure the continuous supply of glucose to insulin-sensitive organs and tissues and to keep the hepatic glucose production at a low level at night.

In contrast to diabetes type 1, there is not generally a lack of insulin in diabetes type 2 but in many cases, particularly in progressive cases, the treatment with insulin is regarded as the most suitable therapy, if required in combination with orally administered anti-diabetic drugs.

An increased glucose level in the blood over several years without initial symptoms represents a significant health risk. It could clearly be shown by the large-scale DCCT study in the USA (The Diabetes Control and Complications Trial Research Group (1993) N. Engl. J. Med. 329, 977-986) that chronically increased levels of blood glucose are a main reason for the development of diabetes complications. Examples for diabetes complications are micro and macrovascular damages that possibly manifest themselves in retinopathies, nephropathies or neuropathies and lead to blindness, renal failure and the loss of extremities and are accompanied by an increased risk of cardiovascular diseases. It can thus be concluded that an improved therapy of diabetes primarily has to aim keeping blood glucose in the physiological range as closely as possible.

A particular risk exists for overweight patients suffering from diabetes type 2, e.g. patients with a body mass index (BMI)≧30. In these patients the risks of diabetes overlap with the risks of overweight, leading e.g. to an increase of cardiovascular diseases compared to diabetes type 2 patients being of a normal weight. Thus, it is particularly necessary to treat diabetes in these patients while reducing the overweight.

Metformin is a biguanide hypoglycemic agent used in the treatment of non-insulin-dependent diabetes mellitus (diabetes mellitus type 2) not responding to dietary modification. Metformin improves glycemic control by improving insulin sensitivity and decreasing intestinal absorption of glucose. Metformin is usually administered orally. However, control diabetes mellitus type 2 in obese patients by metformin may be insufficient. Thus, in these patients, additional measures for controlling diabetes mellitus type 2 may be required.

The compound desPro³⁶Exendin-4(1-39)-Lys₆-NH₂ (AVE0010, lixisenatide) is a derivative of Exendin-4. AVE0010 is disclosed as SEQ ID NO:93 in WO 01/04156:

SEQ ID NO: 1: AVE0010 (44 AS)
H-G-E-G-T-F-T-S-D-L-S-K-Q-M-E-E-E-A-V-R-L-F-I-E-W-
L-K-N-G-G-P-S-S-G-A-P-P-S-K-K-K-K-K-K-NH₂
SEQ ID NO: 2: Exendin-4 (39 AS)
H-G-E-G-T-F-T-S-D-L-S-K-Q-M-E-E-E-A-V-R-L-F-I-E-W-
L-K-N-G-G-P-S-S-G-A-P-P-P-S-NH₂

Exendins are a group of peptides which can lower blood glucose concentration. The Exendin analogue AVE0010 is characterised by C-terminal truncation of the native Exendin-4 sequence. AVE0010 comprises six C-terminal lysine residues not present in Exendin-4.

In the context of the present invention, AVE0010 includes pharmaceutically acceptable salts thereof. The person skilled in the art knows pharmaceutically acceptable salts of AVE0010. A preferred pharmaceutically acceptable salt of AVE0010 employed in the present invention is acetate.

In the Example of the present invention, it was demonstrated that AVE0010 (Lixisenatide) in an add-on therapy to metformin can be effectively administered 1 hour before an evening meal or 1 hour before a morning meal. Significantly improved glycemic control and decreased weight were observed:
HbA1c was significantly decreased.
Postprandial glucose control and glucose excursion was improved.
Significantly more lixisenatide patients achieved HbA1c targets.
Fasting plasma glucose (FPG) was significantly improved with lixisenatide.
Significant weight loss was induced.

A first aspect of the present invention is a pharmaceutical combination for use in the treatment of a diabetes type 2 patient, said combination comprising
(a) desPro³⁶Exendin-4(1-39)-Lys₆-NH₂ or/and a pharmaceutically acceptable salt thereof, and
(b) metformin or/and a pharmaceutically acceptable salt thereof,
   wherein the compound (a) is administered once daily before an evening meal.

In the context of the present invention, “administration before an evening meal” in particular refers to administration in a range from about 4 h, from about 3 h, from about 2 h, from about 1 h 30 min to about 15 min, to about 30 min, or to about 40 min before the evening meal, or about 1 hour before the evening meal.

Preferred is an administration in a range from about 2 h or from about 1 h 30 min, to about 30 min before the evening meal. More preferred is administration about 1 hour before the evening meal.

A further aspect of the present invention is a pharmaceutical combination for use in the treatment of a diabetes type 2 patient, said combination comprising
(a) desPro³⁶Exendin-4(1-39)-Lys₆-NH₂ or/and a pharmaceutically acceptable salt thereof, and
(b) metformin or/and a pharmaceutically acceptable salt thereof,
   wherein the compound (a) is administered once daily before a morning meal.

n the context of the present invention, “administration before a morning meal” in particular refers to administration in a range from about 4 h, from about 3 h, from about 2 h, from about 1 h 30 min to about 15 min, to about 30 min, or to about 40 min before the morning meal, or about 1 hour before the morning meal.

Preferred is an administration in a range from about 2 h or from about 1 h 30 min, to about 30 min before the morning meal. More preferred is administration about 1 hour before the morning meal.

In the present invention, metformin can be administered according to commonly known administration protocols of metformin. For example, metformin can be administrated once daily or twice daily.

Metformin is the international nonproprietary name of 1,1-dimethylbiguanide (CAS Number 657-24-9). In the present invention, the term “metformin” includes any pharmaceutically acceptable salt thereof.

In the present invention, metformin may be administered orally. The skilled person knows formulations of metformin suitable for treatment of diabetes type 2 by oral administration. Metformin may be administered to a subject in need thereof, in an amount sufficient to induce a therapeutic effect. Metformin may be administered in a dose of at least 1.0 g/day or at least 1.5 g/day. For oral administration, metformin may be formulated in a solid dosage form, such as a tablet or pill. Metformin may be formulated with suitable pharmaceutically acceptable carriers, adjuvants, or/and auxiliary substances.

In the present invention, desPro³⁶Exendin-4(1-39)-Lys₆-NH₂ or/and a pharmaceutically acceptable salt may be administered in an add-on therapy to administration of metformin.

In the present invention, the terms “add-on”, “add-on treatment” and “add-on therapy” relate to treatment of diabetes mellitus type 2 with metformin and AVE0010. Metformin and AVE0010 may be administered within a time interval of 24 h. Metformin and AVE0010 each may be administered in a once-a-day-dosage. Metformin and AVE0010 may be administered by different administration routes. Metformin may be administered orally, and AVE0010 may be administered parenterally.

The subject to be treated by the medicament of the present invention suffering from diabetes type 2 may be a subject suffering from diabetes type 2, wherein diabetes type 2 is not adequately controlled by treatment with metformin alone, for instance with a dose of at least 1.0 g/day metformin or at least 1.5 g/day metformin for 3 months. In the present invention, a subject the diabetes type 2 of which is not adequately controlled may have a HbA1c value in the range of 7% to 10%.

The subject to be treated by the medicament of the present invention suffering from diabetes type 2 may be an obese subject. In the present invention, an obese subject may have a body mass index of at least 30 kg/m².

The subject to be treated by the medicament of the present invention suffering from diabetes type 2 may have a normal body weight. In the present invention, a subject having normal body weight may have a body mass index in the range of 17 kg/m² to 25 kg/m², or 17 kg/m² to <30 kg/m².

The subject to be treated by the medicament of the present invention may be an adult subject. The subject may have an age of at least 18 years of may have an age in the range of 18 to 80 years, of 18 to 50 years, or 40 to 80 years, or 50 to 60 years. The subject may be younger than 50 years.

The subject to be treated by the medicament of the present invention preferably does not receive an antidiabetic treatment, for instance by insulin or/and related compounds.

The subject to be treated by the medicament of the present invention may suffer from diabetes mellitus type 2 for at least 1 year or at least 2 years. In particular, in the subject to be treated, diabetes mellitus type 2 has been diagnosed at least 1 year or at least 2 years before onset of therapy by the medicament of the present invention.

The subject to be treated may have a HbA_{1c} value of at least about 8% or at least about 7.5%. The subject may also have a HbA_{1c} value of about 7 to about 10%. The example of the present invention demonstrates that treatment by AVE0010 results in a reduction of the HbA_{1c} value in diabetes type 2 patients.

In yet another aspect of the present invention, the combination as described herein can be used for improving glycemic control. In the present invention, improvement of glycemic control in particular refers to improvement of postprandial plasma glucose concentration, improvement of fasting plasma glucose concentration, or/and improvement of the HbA_{1c} value.

In yet another aspect of the present invention, the combination as described herein can be used for improving the HbA_{1c} value in a patient suffering from diabetes type 2. Improving the HbA_{1c} value means that the HbA_{1c} value is reduced below 6.5% or 7%, for example after treatment for at least one month, at least two months, or at least three months.

In yet another aspect of the present invention, the combination as described herein can be used for improving glucose tolerance in a patient suffering from diabetes type 2. Improving glucose tolerance means that the postprandial plasma glucose concentration is reduced by the active agent of the present invention. Reduction means in particular that the plasma glucose concentration reaches normoglycemic values or at least approaches these values.

In the present invention, normoglycemic values are blood glucose concentrations of in particular 60-140 mg/dl (corresponding to 3,3 bis 7.8 mM/L). This range refers in particular to blood glucose concentrations under fasting conditions and postprandial conditions.

The subject to be treated may have a 2 hours postprandial plasma glucose concentration of at least 10 mmol/L, at least 12 mmol/L, or at least 14 mmol/L. These plasma glucose concentrations exceed normoglycemic concentrations.

The subject to be treated may have a glucose excursion of at least 2 mmol/L, at least 3 mmol/L, at least 4 mmol/L or at least 5 mmol/L. In the present invention, the glucose excursion is in particular the difference of the 2 hours postprandial plasma glucose concentration and the plasma glucose concentration 30 minutes prior to a meal test.

“Postprandial” is a term that is well known to a person skilled in the art of diabetology. The term “postprandial” describes in particular the phase after a meal or/and exposure to glucose under experimental conditions. In a healthy person this phase is characterised by an increase and subsequent decrease in blood glucose concentration. The term “postprandial” or “postprandial phase” typically ends up to 2 h after a meal or/and exposure to glucose.

The subject to be treated as disclosed herein may have a fasting plasma glucose concentration of at least 8 mmol/L, at least 8.5 mmol/L or at least 9 mmol/L. These plasma glucose concentrations exceed normoglycemic concentrations.

In another aspect of the present invention, the combination as described herein can be used for improving (i.e. reducing) fasting plasma glucose in a patient suffering from diabetes type 2. Reduction means in particular that the plasma glucose concentration reaches normoglycemic values or at least approaches these values.

The combination of the present invention can be used in the treatment of one or more of the medical indications described herein, for example in treatment of diabetes type 2 patients, as described herein, or for conditions associated with diabetes type 2, such as improvement of glycemic control, reduction of the fasting plasma glucose concentration, for the improvement of glucose excursion, reduction of the postprandial plasma glucose concentration, improvement of glucose tolerance, improving the HbA_{1c} value, weight loss or/and prevention of weight gain.

In the present invention, desPro³⁶Exendin-4(1-39)-Lys₆-NH₂ or/and the pharmaceutically acceptable salt thereof may be administered to a subject in need thereof, in an amount sufficient to induce a therapeutic effect.

In the present invention, desPro³⁶Exendin-4(1-39)-Lys₆-NH₂ or/and the pharmaceutically acceptable salt thereof may be formulated with suitable pharmaceutically acceptable carriers, adjuvants, or/and auxiliary substances.

The compound desPro³⁶Exendin-4(1-39)-Lys₆-NH₂ or/and a pharmaceutically acceptable salt thereof may be administered parenterally, e.g. by injection (such as by intramuscular or by subcutaneous injection). Suitable injection devices, for instance the so-called “pens” comprising a cartridge comprising the active ingredient, and an injection needle, are known. The compound desPro³⁶Exendin-4(1-39)-Lys₆-NH₂ or/and a pharmaceutically acceptable salt thereof may be administered in a suitable amount, for instance in an amount in the range of 10 to 15 μg per dose or 15 to 20 μg per dose.

In the present invention, desPro³⁶Exendin-4(1-39)-Lys₆-NH₂ or/and a pharmaceutically acceptable salt thereof may be administered in a daily dose in the range of 10 to 20 μg, in the range of 10 to 15 μg, or in the range of 15 to 20 μg. DesPro³⁶Exendin-4(1-39)-Lys₆-NH₂ or/and a pharmaceutically acceptable salt thereof may be administered by one injection per day.

In the present invention, desPro³⁶Exendin-4(1-39)-Lys₆-NH₂ or/and a pharmaceutically acceptable salt thereof may be provided in a liquid composition. The skilled person knows liquid compositions of AVE0010 suitable for parenteral administration. A liquid composition of the present invention may have an acidic or a physiologic pH. An acidic pH preferably is in the range of pH 1-6.8, pH 3.5-6.8, or pH 3.5-5. A physiologic pH preferably is in the range of pH 2.5-8.5, pH 4.0-8.5, or pH 6.0-8.5. The pH may be adjusted by a pharmaceutically acceptable diluted acid (typically HCl) or pharmaceutically acceptable diluted base (typically NaOH).

The liquid composition comprising desPro³⁶Exendin-4(1-39)-Lys₆-NH₂ or/and a pharmaceutically acceptable salt thereof may comprise a suitable preservative. A suitable preservative may be selected from phenol, m-cresol, benzyl alcohol and p-hydroxybenzoic acid ester. A preferred preservative is m-cresol.

The liquid composition comprising desPro³⁶Exendin-4(1-39)-Lys₆-NH₂ or/and a pharmaceutically acceptable salt thereof may comprise a tonicity agent. A suitable tonicity agent may be selected from glycerol, lactose, sorbitol, mannitol, glucose, NaCl, calcium or magnesium containing compounds such as CaCl₂. The concentration of glycerol, lactose, sorbitol, mannitol and glucose may be in the range of 100-250 mM. The concentration of NaCl may be up to 150 mM. A preferred tonicity agent is glycerol.

The liquid composition comprising desPro³⁶Exendin-4(1-39)-Lys₆-NH₂ or/and a pharmaceutically acceptable salt thereof may comprise methionine from 0.5 μg/mL to 20 μg/mL, preferably from 1 μg/ml to 5 μg/ml. Preferably, the liquid composition comprises L-methionine.

A further aspect of the present invention is a pharmaceutical combination as disclosed herein for use in inducing weight loss in diabetes type 2 patients or/and for preventing weight gain in diabetes type 2 patients.

A further aspect of the present invention is a method for inducing weight loss in diabetes type 2 patients or/and for preventing weight gain in diabetes type 2 patients, said method comprising administering desPro³⁶Exendin-4(1-39)-Lys₆-NH₂ or/and a pharmaceutically acceptable salt thereof, in combination with metformin to a subject in need thereof. In particular, the combination as described herein may be administered. In the method of the present invention, the subject may be the subject defined herein.

A further aspect of the present invention is a method for treatment of diabetes type 2 patients, said method comprising administering desPro³⁶Exendin-4(1-39)-Lys₆-NH₂ or/and a pharmaceutically acceptable salt thereof, in combination with metformin to a subject in need thereof, wherein desPro³⁶Exendin-4(1-39)-Lys₆-NH₂ or/and a pharmaceutically acceptable salt thereof is administered once daily before an evening meal. In particular, the combination as described herein may be administered. In the method of the present invention, the subject may be the subject defined herein.

A further aspect of the present invention is a method for treatment of diabetes type 2 patients, said method comprising administering desPro³⁶Exendin-4(1-39)-Lys₆-NH₂ or/and a pharmaceutically acceptable salt thereof, in combination with metformin to a subject in need thereof wherein desPro³⁶Exendin-4(1-39)-Lys₆-NH₂ or/and a pharmaceutically acceptable salt thereof is administered once daily before a morning meal. In particular, the combination as described herein may be administered. In the method of the present invention, the subject may be the subject defined herein.

Yet another aspect of the present invention refers to the use of the combination as described herein for the manufacture of a medicament for the treatment of a medical indication, as described herein. For example, the combination as described herein can be used for the manufacture of a medicament for the treatment of a diabetes type 2 patient, wherein the compound (a), as described herein, is administered once daily before an evening meal. In another example, the combination as described herein can be used for the manufacture of a medicament for the treatment of a diabetes type 2 patient, wherein the compound (a), as described herein, is administered once daily before a morning meal. In another example, the combination as described herein can be used for the manufacture of a medicament for inducing weight loss in diabetes type 2 patients or/and for preventing weight gain in diabetes type 2 patients. The combination of the present invention can also be used for the manufacture of a medicament for the treatment of diabetes type 2 patients, or for the treatment of conditions associated with diabetes type 2, such as improvement of glycemic control, reduction of the fasting plasma glucose concentration, for the improvement of glucose excursion, reduction of the postprandial plasma glucose concentration, improving the HbA_{1c} value, or/and improvement of glucose tolerance. The medicament can be formulated as described herein. For example the medicament can comprise a parenteral formulation of AVE0010 or/and a pharmaceutically acceptable salt thereof, and an oral formulation of metformin or/and a pharmaceutically acceptable salt thereof.

The invention is further illustrated by the following example and figures.

### FIGURE LEGENDS

FIG. 1—Study design

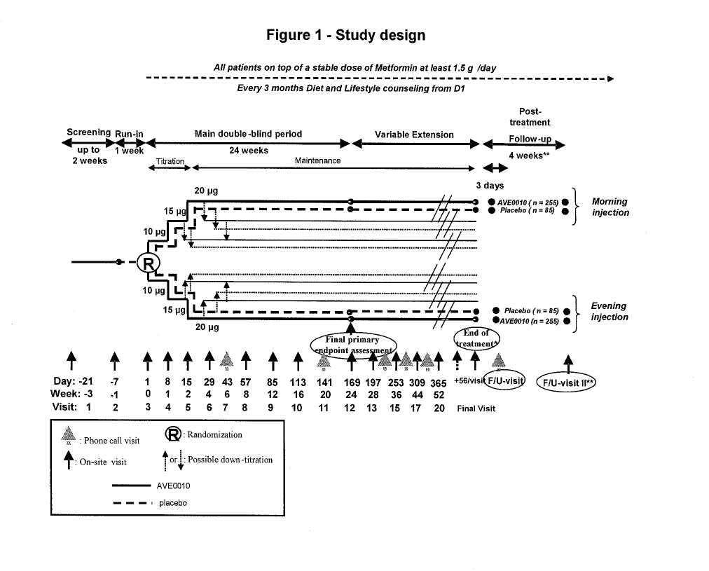

FIG. 2—Step-down testing procedure

FIG. 3—Kaplan-Meier plot of time to treatment discontinuation due to any reason—Randomized population

FIG. 4—Plot of mean change in HbA1c (%) from baseline by visit up to Week 24—mITT population

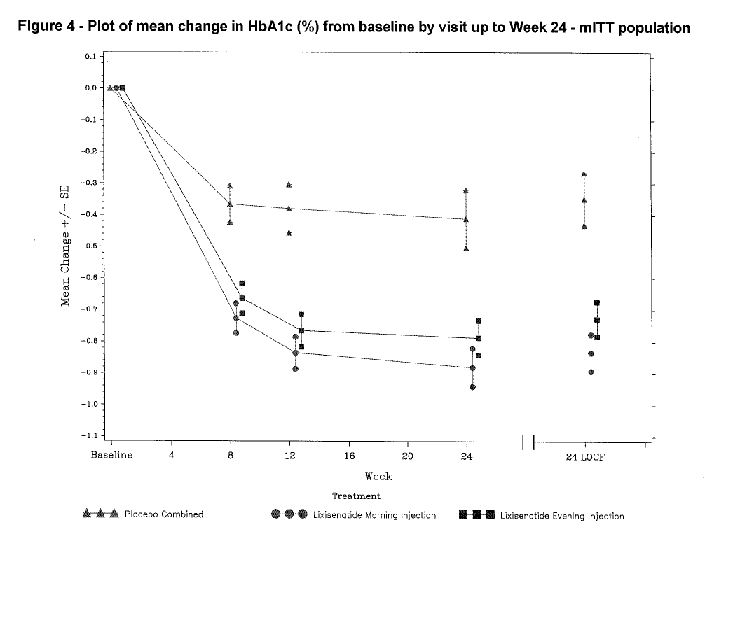

FIG. 5—Plot of mean change in fasting plasma glucose (mmol/L) from baseline by visit up to Week 24—mITT population

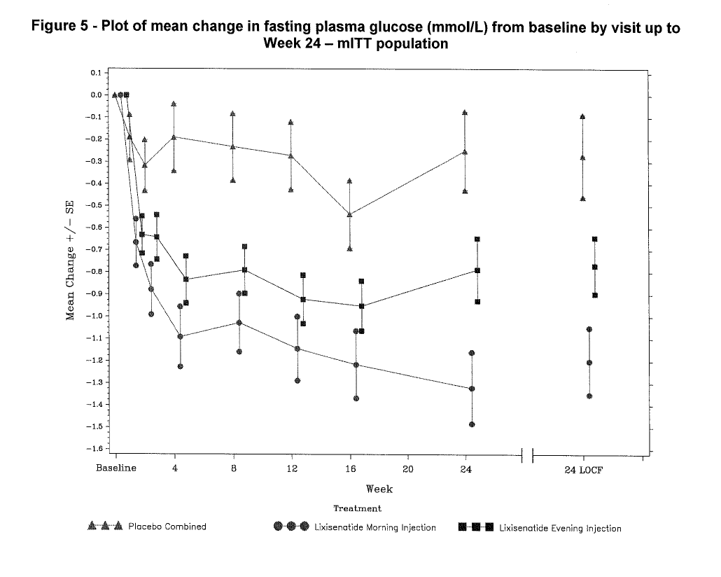

FIG. 6—Plot of mean change in body weight (kg) from baseline by visit up to Week 24—mITT population

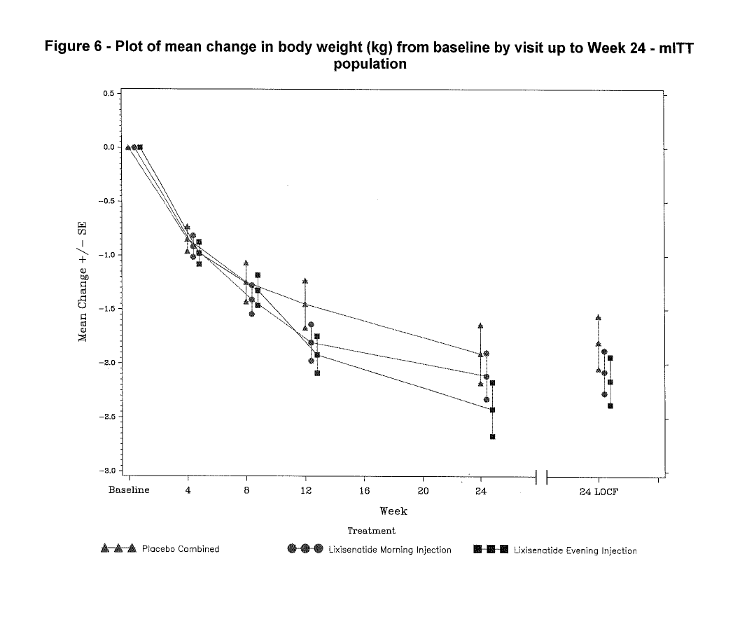

FIG. 7—Plot of mean change in HbA1c (%) from baseline by visit—mITT population

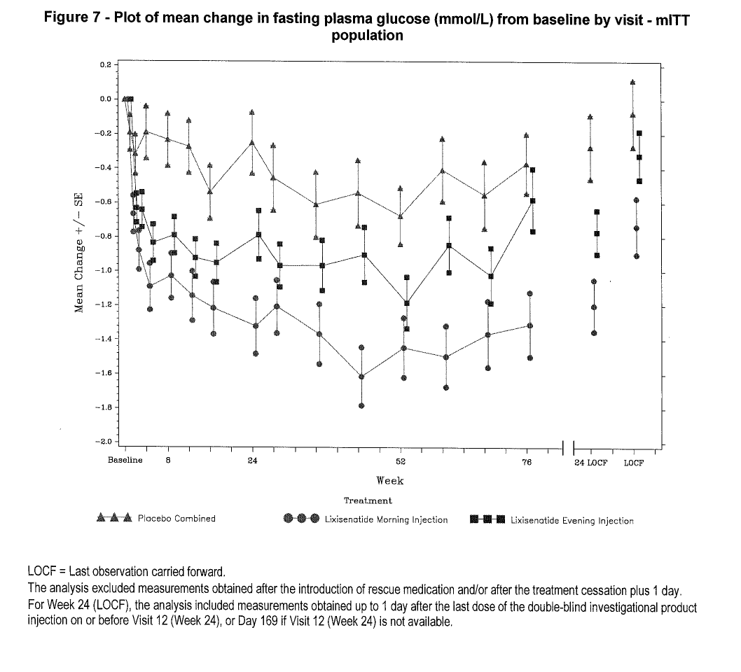

FIG. 8—Plot of mean change in fasting plasma glucose (mmol/L) from baseline by visit—mITT population

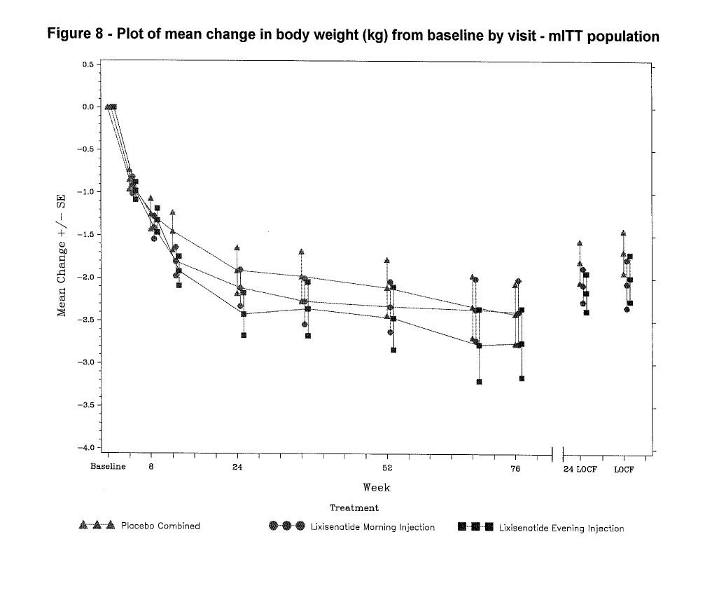

FIG. 9—Plot of mean change in body weight (kg) from baseline by visit—mITT population

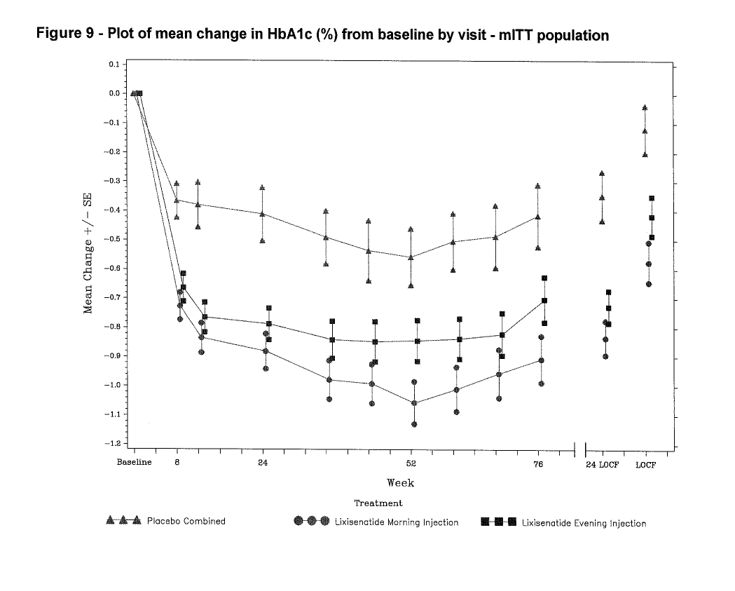

### EXAMPLE

A Randomized, Double-Blind, Placebo-Controlled, 4-Arm, Unbalanced Design, Parallel-Group, Multicenter, Multinational Study Assessing the Efficacy and Safety of Lixisenatide on Top of Metformin in Patients with Type 2 Diabetes, not Adequately Controlled with Metformin

### Summary

A randomized, double-blind, placebo-controlled, 4-arm, unbalanced design, parallel-group, multicenter, multinational study assessing the efficacy and safety of lixisenatide on top of metformin in patients with type 2 diabetes, not adequately controlled with metformin. The approximate minimum study duration per patient was 79 weeks (up to 2 weeks screening+1 week run-in +24-week main double-blind treatment+variable extension+3 days follow-up). A 4-week post-treatment follow-up was performed in patients from the morning injection arms. The extension period ended for all patients approximately at the scheduled date of week 76 visit (V25) for the last randomized patient.

The study was conducted in 133 centers in 16 countries. The primary objective of the study was to assess the efficacy of lixisenatide injected in the morning within 1 hour prior to breakfast on glycemic control in comparison to placebo in terms of HbA_{1c} reduction (absolute change) over a period of 24 weeks.

A total of 680 patients were randomized to one of four treatment arms (255 in each lixisenatide morning and evening injection arm and 85 in each placebo morning and evening injection arm). All randomized patients were exposed to the study treatment and included in the modified intent-to-treat (mITT) population. The placebo morning and evening injection arms were combined in the analyses. Demographics and baseline characteristics were generally similar across the treatment aims with fewer Hispanic and female patients in the combined placebo group. During the whole study treatment period, 169 (24.9%) patients prematurely discontinued the study treatment with a higher percentage in the lixisenatide evening injection arm (27.5%) and a lower percentage in the lixisenatide morning injection arm (22.4%) compared to the combined placebo group (24.7%). In lixisenatide-treated patients, the main reason for treatment discontinuation was “adverse events” (10.2% for evening injection and 8.2% for morning injection versus 3.5% for combined placebo) followed by “other reasons” (8.6% for each lixisenatide arm versus 11.2% for combined placebo).

Efficacy analyses are based on 24-week treatment. The least squared (LS) mean changes from baseline to Week 24 in HbA_{1c} were −0.87% in the lixisenatide morning injection aim. (LS mean difference vs. combined placebo=−0.48%, p-value=<0.0001), and −0.75% in the lixisenatide evening injection arm (LS mean difference vs. combined placebo=−0.37%, p-value=<0.0001), in comparison to −0.38% in the combined placebo group. The percentages of patients reaching HbA_{1c}≦6.5 or <7% at week 24 were significantly higher in both lixisenatide arms than in the combined placebo group (for HbA_{1c}≦6.5%, 23.8% in the lixisenatide morning injection arm and 19.2% in the lixisenatide evening injection arm, versus 10.4% in the combined placebo group; for HbA_{1c}<7%, 43% in the lixisenatide morning injection arm and 40.6% in the lixisenatide evening injection arm, versus 22% in the combined placebo group).

Treatment with lixisenatide also improved post-prandial glycemic control as shown by the results for 2-hour Post-Prandial Glucose (PPG) and for glucose excursion in the morning injection arms (meal test was not performed in the evening injection arms). 2-hour PPG was significantly decreased from baseline to Week 24 in the lixisenatide arm, compared to the placebo arm with a LS mean difference of −4.51 mmol/L (p-value<0.0001). Both lixisenatide arms demonstrated a statistically significant reduction from baseline to Week 24 in Fasting Plasma Glucose (FPG) compared to the combined placebo group (for lixisenatide morning injection, LS mean difference=−0.94 mmol/L, p-value=<0.0001; for lixisenatide evening injection, LS mean difference=−0.56 mmol/L, p-value=0.0046). The LS mean decrease in body weight was 2.01 kg in the lixisenatide morning injection arm and 2.02 kg in the lixisenatide evening injection arm, compared to 1.64 kg in the combined placebo group, with no significant difference observed. Per the testing strategy for multiplicity adjustment, the inferential testing for the subsequent efficacy variables was exploratory since the body weight analysis failed to show a statistically significant difference. A noticeable improvement in β-cell function assessed by HOMA-β was observed in both lixisenatide arms. The LS mean difference was 12.12 (p-value=0.0002 without adjustment for multiplicity) in the lixisenatide morning injection arm and 8.96 (p-value=0.0071 without adjustment for multiplicity) in the lixisenatide evening injection arm, when compared to the combined placebo group. In addition, both lixisenatide arms had substantially lower rates of patients requiring rescue therapy during the main 24-week double-blind treatment period (2.7% for morning injection and 3.9% for evening injection), compared to the combined placebo group (10.6%). No clinically relevant difference in Fasting Plasma Insulin (FPI) was observed between each lixisenatide arm and the combined placebo group.

Safety analyses are based on whole study treatment. Lixisenatide was well tolerated. The incidence of treatment emergent adverse events (TEAEs) was higher in lixisenatide arms (84.7% for morning injection and 83.5% for evening injection), compared to the combined placebo group (75.3%). One patient in the lixisenatide morning injection arm had a TEAE of pancreatic carcinoma leading to death. Two patients in the lixisenatide evening arm died due to post-treatment AEs (haemothorax and lymphoma respectively). A total of 58 patients had at least one serious TEAE, with higher rate in the lixisenatide evening injection arm (10.2%), followed by the lixisenatide morning injection arm (8.2%) and the combined placebo group (6.5%). The most commonly reported TEAE for lixisenatide-treated patients was nausea (64 [25.1%] patients for morning injection and 63 [24.7%] for evening injection, versus 16 [9.4%] for the combined placebo) followed by headache (49 [19.2%] patients for morning injection and 42 [16.5%] for evening injection, versus 28 [16.5%] for the combined placebo). Diarrhoea was reported in 39 (15.3%) patients for morning injection and 36 (14.1%) for evening injection, versus 20 (11.8%) for the combined placebo; and vomiting in 35 (13.7%) patients for morning injection and 40 (15.7%) for evening injection, versus 9 (5.3%) for the combined placebo. Eighteen (7.1%) patients in the lixisenatide morning injection arm and 22 (8.6%) in the lixisenatide evening injection arm had symptomatic hypoglycemia events per protocol definition, compared to 4 (2.4%) placebo-treated patients. None of the symptomatic hypoglycemia events were severe in intensity. A total of 10 patients (3 [1.2%] for lixisenatide morning injection, 4 [1.6%] for lixisenatide evening injection, and 3 [1.8%] for combined placebo) had reported 12 TEAEs that were adjudicated as allergic reactions by ARAC. Of these, 3 events (anaphylactic reaction and angioedema in one patient in the lixisenatide morning injection arm and urticaria in one patient in the lixisenatide evening injection arm) were adjudicated as possibly related to IP. No case of acute pancreatitis was reported in the study. There was no clinically relevant difference in terms of safety and tolerability between morning and evening injection regimen for lixisenatide.

### 1 Objectives

1.1 Primary Objective

The primary objective of this study was to assess the efficacy of lixisenatide on glycemic control when it was used in the morning within 1 hour prior to the meal in comparison to placebo as an add-on treatment to metformin in term's of HbA_{1c} reduction (absolute change) over a period of 24 weeks in patients with type 2 diabetes, not adequately controlled with metformin.

1.2 Key Secondary Objective(s)

The secondary objectives of this study were:
To assess the effect of lixisenatide on glycemic control when administered in the evening within 1 hour prior to the meal in comparison to placebo in terms of HbA_{1c} reduction.
To assess the effects of lixisenatide on:
Percentage of patients reaching HbA_{1c}<7% or HbA_{1c}≦6.5%,
2-hour post-prandial plasma glucose (PPG) in morning injection arms,
Fasting plasma glucose (FPG),
Body weight,
β-cell function assessed by HOMA-β,
Fasting plasma insulin (FPI).
To assess lixisenatide safety and tolerability.

### 2 Trial Design

This was a randomized, double-blind, placebo-controlled, 4-arm, unbalanced design (3:1:3:1), parallel-group, multicenter, multinational study: morning injection (255 lixisenatide treated vs. 85 placebo treated patients) and evening injection (255 lixisenatide vs. 85 placebo treated patients). The study was double-blind with regard to active and placebo treatments. The study drug volume (i.e. dose of active drug or matching placebo) and the time of injection (morning vs. evening) were not blinded. The patients were stratified by screening values of HbA_{1c} (<8%, ≧8%) and body mass index (BMI) (<30 kg/m², ≧30 kg/m²).

The approximate minimum double-blind study duration per patient was 79 weeks (up to 2 weeks screening+1 week run-in +24 weeks main double-blind treatment+variable extension+3 days follow-up). A 4-week follow-up was performed in patients from the morning injection arms only. Patients who completed the 24-week main double-blind period underwent a variable double-blind extension period, which ended for all patients approximately at the scheduled date of week 76 visit (V25) for the last randomized patient.

The standardized meal challenge test was performed in patients in the morning injection arms only.

### 3 Primary and Key Secondary Endpoints

3.1 Primary Endpoint

The primary efficacy variable was the absolute change in HbA_{1c} from baseline to Week 24, which is defined as: HbA_{1c} value at Week 24—HbA_{1c} value at baseline.

If a patient permanently discontinued the treatment, or received rescue therapy during the main 24-week double-blind treatment period, or did not have HbA_{1c} value at Week 24, the last post-baseline on-treatment HbA_{1c} measurement during the main 24-week double-blind on-treatment period was used as HbA_{1c} value at Week 24 (last observation carried forward [LOCF] procedure).

3.2 Secondary Endpoints

3.2.1 Key Efficacy Endpoints

For secondary efficacy variables, the same procedure for handling missing assessments/early discontinuation was applied as for the primary efficacy variable.

### Continuous Variables

Change in 2-hour PPG (mmol/L) after a standardized meal from baseline to Week 24 for the morning injection arms only,
Change in FPG (mmol/L) from baseline to Week 24,
Change in body weight (kg) from baseline to Week 24,
Change in 13-cell function assessed by HOMA-β from baseline to Week 24,
Change in FPI (pmol/L) from baseline to Week 24,
Change in glucose excursion (2-hour PPG-plasma glucose 30 minutes prior to the meal test before study drug administration) after a standardized meal from baseline to Week 24 in the morning injection arms.

### Categorical Variables

Percentage of patients with HbA_{1c}<7% at Week 24,
Percentage of patients with HbA_{1c}≦6.5% at Week 24,
Percentage of patients requiring rescue therapy during the main 24-week double-blind treatment period,
Percentage of patients with ≧5% weight loss (kg) from baseline to Week 24.

3.2.2 Safety Endpoints

The safety analysis was based on the reported TEAEs and other safety information including symptomatic hypoglycemia and severe symptomatic hypoglycemia, local tolerability at injection site, allergic events (as adjudicated by ARAC), suspected pancreatitis, increased calcitonin, vital signs, 12-lead ECG and laboratory tests.

Major cardiovascular events were also collected and adjudicated by a Cardiovascular events Adjudication Committee (CAC). The adjudicated and confirmed events by CAC from this study and other lixisenatide phase 3 studies will be pooled for analyses and summarized in a separate report based on the statistical analysis plan for the overall cardiovascular assessment of lixisenatide. The KRM/CSR will not present the summary of the adjudicated and confirmed CV events from this study.

### 4 Sample Size Calculation Assumptions

The sample size/power calculations were performed based on the primary efficacy variable, absolute change from baseline to week 24 in HbA_{1c}.

A total of 680 patients (255 in each lixisenatide morning or evening injection arm and 85 in each placebo morning or evening injection arm) provided a power of 97% (or 87%) to detect a difference of 0.5% (or 0.4%) in the absolute change in HbA_{1c} from baseline to Week 24 between lixisenatide and placebo. This calculation assumed a common standard deviation of 1.3% with a 2-sided test at the 5% significance level. The sample size calculations were based upon the 2-sample t test and made using nQuery® Advisor 5.0. Standard deviation was estimated in a conservative manner from previously conducted diabetes studies (based on published data of similarly designed study and on internal data, not published), taking into account early dropout.

### 5 Statistical Methods

5.1 Analysis Populations

The modified intent-to-treat (mITT) population consisted of all randomized patients who received at least one dose of double-blind investigational product (IP), and had both a baseline assessment and at least one post-baseline assessment of efficacy variables.

The safety population was defined as all randomized patients who took at least one dose of the double-blind IP.

5.2 Primary Efficacy Analysis

The primary efficacy variable (change in HbA_{1c} from baseline to Week 24) was analyzed using an analysis of covariance (ANCOVA) model with treatment arms (morning injection lixisenatide and placebo arms, evening injection lixisenatide and placebo arms), randomization strata of screening HbA_{1c} (<8.0, ≧8.0%), randomization strata of screening BMI (<30, ≧30 kg/m²) values, and country as fixed effects and using the baseline HbA_{1c} values as a covariate. Differences between each lixisenatide arm and the placebo combined group and its two-sided 95% confidence intervals as well as p-value were estimated within the framework of ANCOVA. In the ANCOVA model, the morning and evening injection placebo arms were included as separate treatments, but combined as one group when presenting results and making comparisons using appropriate contrast (e.g., [−0.5, −0.5, 1, 0] in the order of placebo morning injection, placebo evening injection, lixisenatide morning injection and lixisenatide evening injection when comparing the lixisenatide morning injection arm with the placebo combined group).

A stepwise testing procedure was applied in order to ensure type I error control. First, morning injection lixisenatide arm was compared to the combined placebo group (primary objective). If the test was statistically significant, the evening injection lixisenatide arm would be compared to the combined placebo group (secondary objective).

The primary analysis of the primary efficacy variable was performed based on the mITT population and the measurements obtained during the main 24-week double-blind on-treatment period for efficacy variables. The main 24-week double-blind on-treatment period for efficacy variables except those from the standardized meal test was defined as the time from the first dose of the double-blind IP up to 3 days (except for FPG, FPI, and HOMA-β by central laboratory, which was up to 1 day) after the last dose of the double-blind IP injection on or before Visit 12/Week 24 visit (or Day 169 if Visit 12/Week 24 visit was missing), or up to the introduction of the rescue therapy, whichever the earliest. The main 24-week double-blind on-treatment period for efficacy variables from the meal challenge test including PPG and glucose excursion was defined as the time from the first dose of the double-blind IP up to the date of the last dose of the double-blind IP injection on or before Visit 12/Week 24 visit (or Day 169 if Visit 12/Week 24 visit was missing), or up to the introduction of the rescue therapy, whichever the earliest. The LOCF procedure was used by taking the last available post-baseline on-treatment HbA_{1c} measurement (before the initiation of the new medication in the event of rescue therapy) as the HbA_{1c} value at week 24.

5.3 Secondary Efficacy Analysis

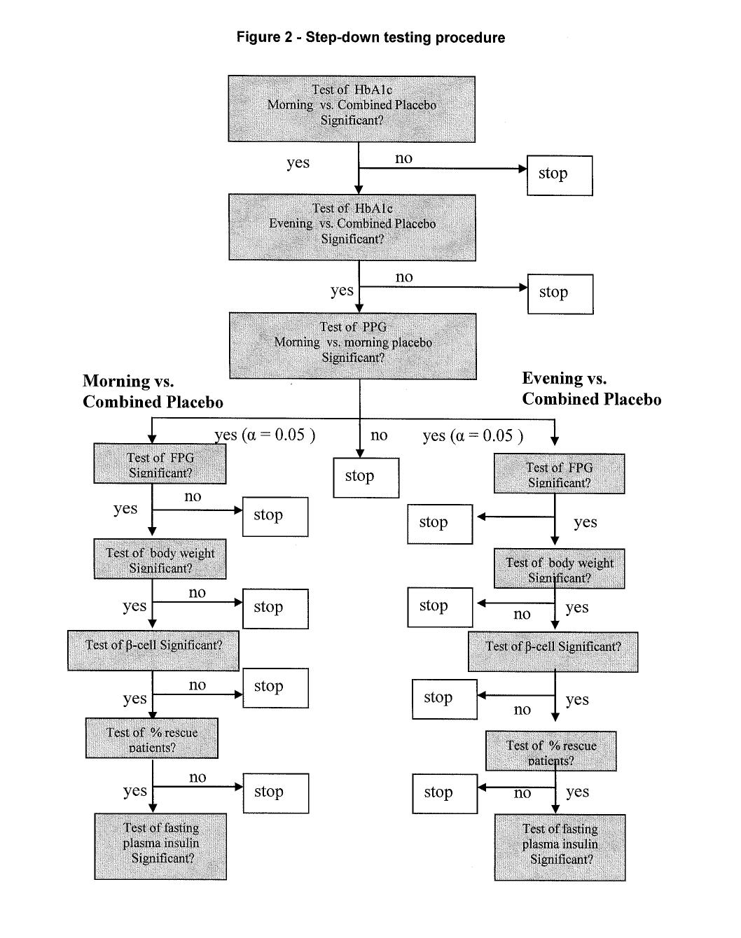

Once the primary efficacy variable was statistically significant at α=0.05 for both comparisons, the testing procedure was performed to test the change in 2-hour PPG (mmol/L) after a standardized meal test from baseline to Week 24 in the morning injection arms, then to test the remaining secondary efficacy variables by the following prioritized order in 2 separate branches: the morning injection arm versus the combined placebo and the evening injection arm versus the combined placebo. The tests stopped as soon as an endpoint was found not statistically significant at α=0.05 (FIG. 2).

All continuous secondary efficacy variables at week 24 as described in Section 3.2.1 were analyzed using the similar approach and ANCOVA model as described in Section 5.2 for the primary analysis of the primary efficacy endpoint. The adjusted estimates of the treatment mean difference between lixisenatide and placebo and two-sided 95% confidence intervals were provided.

The following categorical secondary efficacy variables at Week 24 were analyzed using a Cochran-Mantel-Haenszel (CMH) method stratified on randomization strata (screening HbA_{1c} [<8.0, ≧8%] and screening BMI [<30, ≧30 kg/m²]):
Percentage of patients with HbA_{1c}<7.0% at Week 24,
Percentage of patients with HbA_{1c}≦6.5% at Week 24,
Percentage of patients requiring rescue therapy during the main 24-week double-blind treatment period.

Number and percentage of patients with ≧5% weight loss from baseline at week 24 were presented by treatment groups.

All secondary endpoints at the end of treatment were only evaluated by descriptive statistics (mean, standard deviation, median and ranges provided in CSR).

5.4 Safety Analysis

The safety analyses were primarily based on the on-treatment period of the whole study. The on-treatment period of the whole study was defined as the time from the first dose of double-blind IP up to 3 days after the last dose of IP administration during the whole study period regardless of rescue status. The 3-day interval was chosen based on the half-life of the IP (approximately 5 times the half-life).

In addition, the safety analyses for the 24-week double-blind treatment period will be summarized in CSR.

The summary of safety results (descriptive statistics or frequency tables) is presented by treatment groups.

### 6 Results

6.1 Study Patients

6.1.1 Patient Accountability

The study was conducted in 133 centers in 16 countries (Australia, Canada, Chile, Czech Republic, Germany, Croatia, Mexico, Morocco, Philippines, Romania, Russian Federation, South Africa, Spain, Ukraine, United States and Venezuela). A total of 1374 patients were screened and 680 were randomized to one of the four treatment arms. The most common reason for non-randomization was HbA_{1c} value out of ranges at the screening visit as defined per protocol (483 [35.2%] out of 1374 screened patients).

All 680 randomized patients were exposed to the study treatment and included in mITT population. Table 1 provides the number of patients included in each analysis population.

TABLE 1
Analysis populations - Randomized population

| | Placebo | | | Lixisenatide | | | |
|---|---|---|---|---|---|---|---|
| | Morning | Evening | | Morning | Evening | | |
| | Injection | Injection | Combined | Injection | Injection | Combined | All |
| Randomized population | 85 (100%) | 85 (100%) | 170 (100%) | 255 (100%) | 255 (100%) | 510 (100%) | 680 (100%) |
| Efficacy populations | | | | | | | |
| Modified Intent-to-Treat | 85 (100%) | 85 (100%) | 170 (100%) | 255 (100%) | 255 (100%) | 510 (100%) | 680 (100%) |
| (mITT) | | | | | | | |
| Safety population | 85 | 85 | 170 | 255 | 255 | 510 | 680 |

Note:
The safety population patients are tabulated according to treatment actually received (as treated).
For the efficacy populations, patients are tabulated according to their randomized treatment (as randomized).

6.1.2 Study Disposition

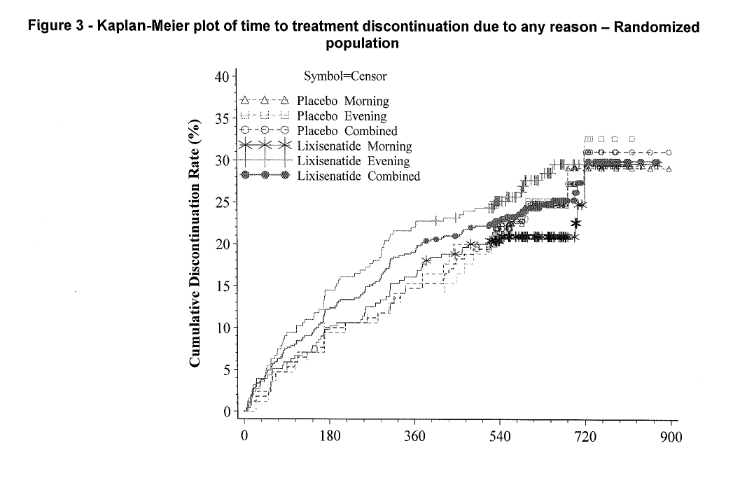

Table 2 provides the summary of patient disposition for each treatment group. During the overall treatment period, 169 (24.9%) patients prematurely discontinued the study treatment with a higher percentage in the lixisenatide evening injection arm (27.5%) and a lower percentage in the lixisenatide morning injection arm (22.4%) compared to the combined placebo group (24.7%). In lixisenatide treated patients, the main reason for treatment discontinuation was “adverse events” (10.2% for evening injection and 8.2% for morning injection versus 3.5% for combined placebo) followed by “other reasons” (8.6% for each lixisenatide arm versus 11.2% for combined placebo). Similar results were observed for the main 24-week treatment period, where a total of 65 (9.6%) patients prematurely discontinued the study treatment (12.2% in the lixisenatide evening injection arm, 8.6% in the lixisenatide morning injection arm versus 7.1% in the combined placebo group) with the main reason also being “adverse events” for lixisenatide arms (5.1% for evening injection and 4.7% for morning injection, versus 1.2% for the combined placebo). The time-to-onset of treatment discontinuation due to any reason for the overall treatment period is depicted in FIG. 3. A higher rate of discontinuation was observed in the lixisenatide evening injection arm during the whole treatment period, as compared to the lixisenatide morning injection arm and the combined placebo group, which looked alike.

Two patients in the lixisenatide evening injection arm were not counted in Table 22 because their treatments were discontinued due to AEs that occurred during the post-treatment period.

TABLE 2
Patient disposition - Randomized population

| | Placebo | | | | | | Lixisenatide | | | | | |
|---|---|---|---|---|---|---|---|---|---|---|---|---|
| | Morning | | Evening | | | | Morning | | Evening | | | |
| | Injection | | Injection | | Combined | | Injection | | Injection | | Combined | |
| | (N = 85) | | (N = 85) | | (N = 170) | | (N = 255) | | (N = 255) | | (N = 510) | |
| Randomized and treated | 85 | (100%) | 85 | (100%) | 170 | (100%) | 255 | (100%) | 255 | (100%) | 510 | (100%) |
| Did not complete 24-week double-blind study | 6 | (7.1%) | 6 | (7.1%) | 12 | (7.1%) | 22 | (8.6%) | 31 | (12.2%) | 53 | (10.4%) |
| treatment | | | | | | | | | | | | |
| Subject's request for 24-week treatment | 5 | (5.9%) | 2 | (2.4%) | 7 | (4.1%) | 17 | (6.7%) | 27 | (10.6%) | 44 | (8.6%) |
| discontinuation | | | | | | | | | | | | |
| Reason for 24-week study treatment discontinuation | 6 | (7.1%) | 6 | (7.1%) | 12 | (7.1%) | 22 | (8.6%) | 31 | (12.2%) | 53 | (10.4%) |
| Adverse event | 1 | (1.2%) | 1 | (1.2%) | 2 | (1.2%) | 12 | (4.7%) | 13 | (5.1%) | 25 | (4.9%) |
| Lack of efficacy | 1 | (1.2%) | 2 | (2.4%) | 3 | (1.8%) | 1 | (0.4%) | 0 | | 1 | (0.2%) |
| Poor compliance to protocol | 1 | (1.2%) | 2 | (2.4%) | 3 | (1.8%) | 2 | (0.8%) | 7 | (2.7%) | 9 | (1.8%) |
| Lost to follow-up | 0 | | 0 | | 0 | | 1 | (0.4%) | 0 | | 1 | (0.2%) |
| Other reasons | 3 | (3.5%) | 1 | (1.2%) | 4 | (2.4%) | 6 | (2.4%) | 11 | (4.3%) | 17 | (3.3%) |
| Did not complete double-blind study treatment | 21 | (24.7%) | 21 | (24.7%) | 42 | (24.7%) | 57 | (22.4%) | 70 | (27.5%) | 127 | (24.9%) |
| Subject's request for treatment discontinuation | 17 | (20.0%) | 16 | (18.8%) | 33 | (19.4%) | 41 | (16.1%) | 58 | (22.7%) | 99 | (19.4%) |
| Reason for study treatment discontinuation | 21 | (24.7%) | 21 | (24.7%) | 42 | (24.7%) | 57 | (22.4%) | 70 | (27.5%) | 127 | (24.9%) |
| Adverse event | 3 | (3.5%) | 3 | (3.5%) | 6 | (3.5%) | 21 | (8.2%) | 26 | (10.2%) | 47 | (9.2%) |
| Lack of efficacy | 2 | (2.4%) | 8 | (9.4%) | 10 | (5.9%) | 8 | (3.1%) | 6 | (2.4%) | 14 | (2.7%) |
| Poor compliance to protocol | 3 | (3.5%) | 3 | (3.5%) | 6 | (3.5%) | 4 | (1.6%) | 16 | (6.3%) | 20 | (3.9%) |
| Lost to follow-up | 1 | (1.2%) | 0 | | 1 | (0.6%) | 2 | (0.8%) | 0 | | 2 | (0.4%) |
| Other reasons | 12 | (14.1%) | 7 | (8.2%) | 19 | (11.2%) | 22 | (8.6%) | 22 | (8.6%) | 44 | (8.6%) |
| Status at last study contact | 85 | (100%) | 85 | (100%) | 170 | (100%) | 255 | (100%) | 255 | (100%) | 510 | (100%) |
| Alive | 83 | (97.6%) | 85 | (100%) | 168 | (98.8%) | 252 | (98.8%) | 251 | (98.4%) | 503 | (98.6%) |
| Dead | 0 | | 0 | | 0 | | 1 | (0.4%) | 2 | (0.8%) | 3 | (0.6%) |
| Lost to follow-up | 2 | (2.4%) | 0 | | 2 | (1.2%) | 2 | (0.8%) | 2 | (0.8%) | 4 | (0.8%) |

Note:
Percentages are calculated using the number of randomized patients as denominator.

6.1.3 Demographics and Baseline Characteristics

The demographic and patient baseline characteristics were generally similar across treatment arms for the safety population (Table 3), with however fewer Hispanic and female patients in the combined placebo group. The median age was 55 years and 56.9% were female. The study population was primarily Caucasian (88.8%). The majority of the patients (65.1%) were obese.

Disease characteristics including diabetic history were generally comparable across treatment arms (Table 4). Across all treatment arms, the median duration of diabetes was 4.74 years and the median age at onset of diabetes was 48 years. On average patients were on metformin for 3.61 years and the median daily metformin dose was 2000 mg.

HbA_{1c}, 2-hour PPG, FPG, body weight, HOMA-β at baseline were generally comparable across treatment arms for the safety population (Table 5). The average HbA_{1c} at baseline was 8.06%.

TABLE 3
Demographics and patient characteristics at screening or baseline - Safety population

| | Placebo | | | | | | Lixisenatide | | | | | | | |
|---|---|---|---|---|---|---|---|---|---|---|---|---|---|---|
| | Morning | | Evening | | | | Morning | | Evening | | | | | |
| | Injection | | Injection | | Combined | | Injection | | Injection | | Combined | | All | |
| | (N = 85) | | (N = 85) | | (N = 170) | | (N = 255) | | (N = 255) | | (N = 510) | | (N = 680) | |
| Age (years) | | | | | | | | | | | | | | |
| Number | 85 | | 85 | | 170 | | 255 | | 255 | | 510 | | 680 | |
| Mean (SD) | 54.5 | (9.8) | 55.5 | (9.0) | 55.0 | (9.4) | 54.5 | (9.2) | 54.8 | (10.4) | 54.6 | (9.8) | 54.7 | (9.7) |
| Median | 55.0 | | 56.0 | | 55.5 | | 55.0 | | 55.0 | | 55.0 | | 55.0 | |
| Min:Max | 25:73 | | 33:76 | | 25:76 | | 33:81 | | 23:87 | | 23:87 | | 23:87 | |
| Age Group | | | | | | | | | | | | | | |
| (years) [n (%)] | | | | | | | | | | | | | | |
| Number | 85 | | 85 | | 170 | | 255 | | 255 | | 510 | | 680 | |
| <50 | 26 | (30.6%) | 17 | (20.0%) | 43 | (25.3%) | 79 | (31.0%) | 77 | (30.2%) | 156 | (30.6%) | 199 | (29.3%) |
| ≧50 to <65 | 44 | (51.8%) | 55 | (64.7%) | 99 | (58.2%) | 147 | (57.6%) | 135 | (52.9%) | 282 | (55.3%) | 381 | (56.0%) |
| ≧65 to <75 | 15 | (17.6%) | 12 | (14.1%) | 27 | (15.9%) | 26 | (10.2%) | 36 | (14.1%) | 62 | (12.2%) | 89 | (13.1%) |
| ≧75 | 0 | | 1 | (1.2%) | 1 | (0.6%) | 3 | (1.2%) | 7 | (2.7%) | 10 | (2.0%) | 11 | (1.6%) |
| Sex [n (%)] | | | | | | | | | | | | | | |
| Number | 85 | | 85 | | 170 | | 255 | | 255 | | 510 | | 680 | |
| Male | 36 | (42.4%) | 45 | (52.9%) | 81 | (47.6%) | 98 | (38.4%) | 114 | (44.7%) | 212 | (41.6%) | 293 | (43.1%) |
| Female | 49 | (57.6%) | 40 | (47.1%) | 89 | (52.4%) | 157 | (61.6%) | 141 | (55.3%) | 298 | (58.4%) | 387 | (56.9%) |
| Race [n (%)] | | | | | | | | | | | | | | |
| Number | 85 | | 85 | | 170 | | 255 | | 255 | | 510 | | 680 | |
| Caucasian/ | 78 | (91.8%) | 77 | (90.6%) | 155 | (91.2%) | 221 | (86.7%) | 228 | (89.4%) | 449 | (88.0%) | 604 | (88.8%) |
| White | | | | | | | | | | | | | | |
| Black | 2 | (2.4%) | 2 | (2.4%) | 4 | (2.4%) | 7 | (2.7%) | 6 | (2.4%) | 13 | (2.5%) | 17 | (2.5%) |
| Asian/Oriental | 5 | (5.9%) | 6 | (7.1%) | 11 | (6.5%) | 22 | (8.6%) | 20 | (7.8%) | 42 | (8.2%) | 53 | (7.8%) |
| Other | 0 | | 0 | | 0 | | 5 | (2.0%) | 1 | (0.4%) | 6 | (1.2%) | 6 | (0.9%) |
| Ethnicity | | | | | | | | | | | | | | |
| [n (%)] | | | | | | | | | | | | | | |
| Number | 85 | | 85 | | 170 | | 255 | | 255 | | 510 | | 680 | |
| Hispanic | 23 | (27.1%) | 26 | (30.6%) | 49 | (28.8%) | 96 | (37.6%) | 98 | (38.4%) | 194 | (38.0%) | 243 | (35.7%) |
| Non Hispanic | 62 | (72.9%) | 59 | (69.4%) | 121 | (71.2%) | 159 | (62.4%) | 157 | (61.6%) | 316 | (62.0%) | 437 | (64.3%) |
| Screening | | | | | | | | | | | | | | |
| HbA1c (%) | | | | | | | | | | | | | | |
| Number | 85 | | 85 | | 170 | | 255 | | 255 | | 510 | | 680 | |
| Mean (SD) | 8.14 | (0.80) | 8.15 | (0.92) | 8.15 | (0.86) | 8.15 | (0.84) | 8.21 | (0.86) | 8.18 | (0.85) | 8.17 | (0.85) |
| Median | 8.00 | | 8.00 | | 8.00 | | 8.00 | | 8.00 | | 8.00 | | 8.00 | |
| Min:Max | 7.0:9.9 | | 7.0:10.0 | | 7.0:10.0 | | 5.7:10.0 | | 7.0:10.0 | | 5.7:10.0 | | 5.7:10.0 | |
| Randomization | | | | | | | | | | | | | | |
| strata of | | | | | | | | | | | | | | |
| screening | | | | | | | | | | | | | | |
| HbA1c | | | | | | | | | | | | | | |
| (%) [n (%)] | | | | | | | | | | | | | | |
| Number | 85 | | 85 | | 170 | | 255 | | 255 | | 510 | | 680 | |
| <8 | 42 | (49.4%) | 42 | (49.4%) | 84 | (49.4%) | 126 | (49.4%) | 126 | (49.4%) | 252 | (49.4%) | 336 | (49.4%) |
| ≧8 | 43 | (50.6%) | 43 | (50.6%) | 86 | (50.6%) | 129 | (50.6%) | 129 | (50.6%) | 258 | (50.6%) | 344 | (50.6%) |
| Screening | | | | | | | | | | | | | | |
| BMI (kg/m²) | | | | | | | | | | | | | | |
| Number | 85 | | 85 | | 170 | | 255 | | 255 | | 510 | | 680 | |
| Mean (SD) | 33.64 | (7.03) | 32.87 | (5.81) | 33.26 | (6.44) | 33.34 | (6.87) | 32.59 | (5.74) | 32.97 | (6.34) | 33.04 | (6.36) |
| Median | 32.20 | | 32.28 | | 32.24 | | 31.68 | | 32.04 | | 31.86 | | 31.93 | |
| Min:Max | 22.7:58.6 | | 22.9:47.8 | | 22.7:58.6 | | 21.0:63.2 | | 21.0:53.3 | | 21.0:63.2 | | 21.0:63.2 | |
| Randomization | | | | | | | | | | | | | | |
| strata of | | | | | | | | | | | | | | |
| screening BMI | | | | | | | | | | | | | | |
| (kg/m²) | | | | | | | | | | | | | | |
| [n (%)] | | | | | | | | | | | | | | |
| Number | 85 | | 85 | | 170 | | 255 | | 255 | | 510 | | 680 | |
| <30 | 30 | (35.3%) | 29 | (34.1%) | 59 | (34.7%) | 89 | (34.9%) | 89 | (34.9%) | 178 | (34.9%) | 237 | (34.9%) |
| ≧30 | 55 | (64.7%) | 56 | (65.9%) | 111 | (65.3%) | 166 | (65.1%) | 166 | (65.1%) | 332 | (65.1%) | 443 | (65.1%) |
| Baseline BMI | | | | | | | | | | | | | | |
| (kg/m²) | | | | | | | | | | | | | | |
| Number | 85 | | 85 | | 170 | | 255 | | 255 | | 510 | | 680 | |
| Mean (SD) | 33.53 | (7.02) | 32.71 | (5.83) | 33.12 | (6.45) | 33.22 | (6.85) | 32.47 | (5.77) | 32.84 | (6.34) | 32.91 | (6.36) |
| Median | 32.04 | | 31.89 | | 31.96 | | 31.60 | | 31.76 | | 31.67 | | 31.80 | |
| Min:Max | 22.6:59.5 | | 22.4:47.3 | | 22.4:59.5 | | 21.0:63.2 | | 20.8:53.4 | | 20.8:63.2 | | 20.8:63.2 | |
| Baseline BMI | | | | | | | | | | | | | | |
| Categories | | | | | | | | | | | | | | |
| (kg/m²) [n (%)] | | | | | | | | | | | | | | |
| Number | 85 | | 85 | | 170 | | 255 | | 255 | | 510 | | 680 | |
| <30 | 29 | (34.1%) | 30 | (35.3%) | 59 | (34.7%) | 95 | (37.3%) | 93 | (36.5%) | 188 | (36.9%) | 247 | (36.3%) |
| ≧30 | 56 | (65.9%) | 55 | (64.7%) | 111 | (65.3%) | 160 | (62.7%) | 162 | (63.5%) | 322 | (63.1%) | 433 | (63.7%) |

BMI = Body Mass Index.

TABLE 4
Disease characteristics at screening or baseline-Safety population

| Placebo | | | Lixisenatide | | | |
|---|---|---|---|---|---|---|
| Morning | Evening | | Morning | Evening | | |
| Injection | Injection | Combined | Injection | Injection | Combined | All |
| (N = 85) | (N = 85) | (N = 170) | (N = 255) | (N = 255) | (N = 510) | (N = 680) |

Duration of diabetes (years)

| | | | | | | | |
|---|---|---|---|---|---|---|---|
| Number | 85 | 85 | 170 | 255 | 255 | 510 | 680 |
| Mean (SD) | 5.62 (4.29) | 6.11 (5.13) | 5.87 (4.72) | 6.18 (5.25) | 6.21 (5.40) | 6.19 (5.32) | 6.11 (5.17) |
| Median | 4.55 | 4.08 | 4.29 | 5.12 | 4.54 | 4.93 | 4.74 |
| Min:Max | 1.1:24.4 | 0.8:23.8 | 0.8:24.4 | 0.9:52.1 | 1.0:29.0 | 0.9:52.1 | 0.8:52.1 |

Age at onset of type 2 diabetes (years)

| | | | | | | | |
|---|---|---|---|---|---|---|---|
| Number | 85 | 85 | 170 | 255 | 255 | 510 | 680 |
| Mean (SD) | 48.81 (9.09) | 49.32 (9.69) | 49.06 (9.37) | 48.32 (8.67) | 48.56 (9.96) | 48.44 (9.33) | 48.60 (9.33) |
| Median | 50.00 | 48.00 | 49.50 | 48.00 | 48.00 | 48.00 | 48.00 |
| Min:Max | 24.0:69.0 | 16.0:72.0 | 16.0:72.0 | 24.0:73.0 | 13.0:80.0 | 13.0:80.0 | 13.0:80.0 |

Duration of metformin treatment (years)

| | | | | | | | |
|---|---|---|---|---|---|---|---|
| Number | 85 | 85 | 170 | 255 | 255 | 510 | 680 |
| Mean (SD) | 3.00 (2.87) | 3.68 (3.93) | 3.34 (3.45) | 3.73 (3.34) | 3.68 (3.90) | 3.71 (3.63) | 3.61 (3.59) |
| Median | 2.06 | 2.25 | 2.21 | 2.64 | 2.17 | 2.39 | 2.35 |
| Min:Max | 0.3:11.8 | 0.3:19.1 | 0.3:19.1 | 0.2:20.5 | 0.3:19.3 | 0.2:20.5 | 0.2:20.5 |

Daily dose of metformin at baseline (mg)

| | | | | | | | |
|---|---|---|---|---|---|---|---|
| Number | 85 | 85 | 170 | 255 | 255 | 510 | 680 |
| Mean (SD) | 2005.00 | 1997.35 | 2001.18 | 1968.82 | 1942.65 | 1955.74 | 1967.10 |
| | (449.98) | (431.81) | (439.70) | (446.99) | (406.17) | (426.85) | (430.22) |
| Median | 1700.00 | 2000.00 | 2000.00 | 1700.00 | 2000.00 | 1925.00 | 2000.00 |
| Min:Max | 1500.0:3000.0 | 1500.0:3000.0 | 1500.0:3000.0 | 1500.0:3000.0 | 1500.0:3000.0 | 1500.0:3000.0 | 1500.0:3000.0 |

Categorized daily dose of metformin at baseline (mg) [n (%)]

| | | | | | | | |
|---|---|---|---|---|---|---|---|
| Number | 85 | 85 | 170 | 255 | 255 | 510 | 680 |
| <1500 | 0 | 0 | 0 | 0 | 0 | 0 | 0 |
| ≧1500-<2500 | 62 (72.9%) | 65 (76.5%) | 127 (74.7%) | 195 (76.5%) | 197 (77.3%) | 392 (76.9%) | 519 (76.3%) |
| ≧2500-<3000 | 17 (20.0%) | 14 (16.5%) | 31 (18.2%) | 42 (16.5%) | 51 (20.0%) | 93 (18.2%) | 124 (18.2%) |
| ≧3000 | 6 (7.1%) | 6 (7.1%) | 12 (7.1%) | 18 (7.1%) | 7 (2.7%) | 25 (4.9%) | 37 (5.4%) |

History of gestational diabetes

| | | | | | | | |
|---|---|---|---|---|---|---|---|
| Number (Female) | 49 | 40 | 89 | 157 | 141 | 298 | 387 |
| Yes (Female) | 0 | 3 (7.5%) | 3 (3.4%) | 11 (7.0%) | 9 (6.4%) | 20 (6.7%) | 23 (5.9%) |
| No (Female) | 49 (100%) | 37 (92.5%) | 86 (96.6%) | 146 (93.0%) | 132 (93.6%) | 278 (93.3%) | 364 (94.1%) |

Prior use of GLP-1 receptor agonist [n (%)]

| | | | | | | | |
|---|---|---|---|---|---|---|---|
| Number | 85 | 85 | 170 | 255 | 255 | 510 | 680 |
| Yes | 2 (2.4%) | 4 (4.7%) | 6 (3.5%) | 4 (1.6%) | 8 (3.1%) | 12 (2.4%) | 18(2.6%) |
| No | 83 (97.6%) | 81 (95.3%) | 164 (96.5%) | 251 (98.4%) | 247 (96.9%) | 498 (97.6%) | 662 (97.4%) |

Diabetic retinopathy [n (%)]

| | | | | | | | |
|---|---|---|---|---|---|---|---|
| Number | 85 | 85 | 170 | 252 | 255 | 507 | 677 |
| Yes | 6 (7.1%) | 6 (7.1%) | 12 (7.1%) | 16 (6.3%) | 18 (7.1%) | 34 (6.7%) | 46 (6.8%) |
| No | 74 (87.1%) | 70 (82.4%) | 144 (84.7%) | 231 (91.7%) | 219 (85.9%) | 450 (88.8%) | 594 (87.7%) |
| Unknown | 5 (5.9%) | 9 (10.6%) | 14 (8.2%) | 5 (2.0%) | 18 (7.1%) | 23 (4.5%) | 37 (5.5%) |

Diabetic sensory or motor neuropathy [n (%)]

| | | | | | | | |
|---|---|---|---|---|---|---|---|
| Number | 85 | 85 | 170 | 252 | 254 | 506 | 676 |
| Yes | 12 (14.1%) | 10 (11.8%) | 22 (12.9%) | 39 (15.5%) | 37 (14.6%) | 76 (15.0%) | 98 (14.5%) |
| No | 70 (82.4%) | 69 (81.2%) | 139 (81.8%) | 211 (83.7%) | 210 (82.7%) | 421 (83.2%) | 560 (82.8%) |
| Unknown | 3 (3.5%) | 6 (7.1%) | 9 (5.3%) | 2 (0.8%) | 7 (2.8%) | 9 (1.8%) | 18 (2.7%) |

Diabetic autonomic neuropathy [n (%)]

| | | | | | | | |
|---|---|---|---|---|---|---|---|
| Number | 85 | 85 | 170 | 252 | 255 | 507 | 677 |
| Yes | 3 (3.5%) | 1 (1.2%) | 4 (2.4%) | 1 (0.4%) | 6 (2.4%) | 7 (1.4%) | 11 (1.6%) |
| No | 77 (90.6%) | 77 (90.6%) | 154 (90.6%) | 247 (98.0%) | 242 (94.9%) | 489 (96.4%) | 643 (95.0%) |
| Unknown | 5 (5.9%) | 7 (8.2%) | 12 (7.1%) | 4 (1.6%) | 7 (2.7%) | 11 (2.2%) | 23 (3.4%) |

Diabetic nephropathy [n (%)]

| | | | | | | | |
|---|---|---|---|---|---|---|---|
| Number | 85 | 85 | 170 | 252 | 255 | 507 | 677 |
| Yes | 1 (1.2%) | 1 (1.2%) | 2 (1.2%) | 9 (3.6%) | 7 (2.7%) | 16 (3.2%) | 18 (2.7%) |
| Microalbuminuria | 1 (1.2%) | 0 | 1 (0.6%) | 8 (3.2%) | 5 (2.0%) | 13 (2.6%) | 14 (2.1%) |
| Overt proteinuria | 0 | 1 (1.2%) | 1 (0.6%) | 0 | 2 (0.8%) | 2 (0.4%) | 3 (0.4%) |
| Impaired renal | 0 | 0 | 0 | 0 | 0 | 0 | 0 |
| function | | | | | | | |
| Dialysis or | 0 | 0 | 0 | 0 | 0 | 0 | 0 |
| transplantation | | | | | | | |
| Unknown | 0 | 0 | 0 | 1 (0.4%) | 0 | 1 (0.2%) | 1 (0.1%) |
| No | 82 (96.5%) | 79 (92.9%) | 161 (94.7%) | 236 (93.7%) | 240 (94.1%) | 476 (93.9%) | 637 (94.1%) |
| Unknown | 2 (2.4%) | 5 (5.9%) | 7 (4.1%) | 7 (2.8%) | 8 (3.1%) | 15 (3.0%) | 22 (3.2%) |

Categorized albuminuria at randomization

| | | | | | | | |
|---|---|---|---|---|---|---|---|
| Number | 20 | 23 | 43 | 55 | 63 | 118 | 161 |
| <3 mg/L | 2 (10.0%) | 4 (17.4%) | 6 (14.0%) | 4 (7.3%) | 3 (4.8%) | 7 (5.9%) | 13 (8.1%) |
| (Not reportable) | | | | | | | |
| ≧ 3mg/L | 18 (90.0%) | 19 (82.6%) | 37 (86.0%) | 51 (92.7%) | 60 (95.2%) | 111 (94.1%) | 148 (91.9%) |
| (Reportable) | | | | | | | |
| <20 mg/L | 15 (75.0%) | 11 (47.8%) | 26 (60.5%) | 34 (61.8%) | 31 (49.2%) | 65 (55.1%) | 91 (56.5%) |
| ≧20-<200 mg/L | 3 (15.0%) | 7 (30.4%) | 10 (23.3%) | 12 (21.8%) | 24 (38.1%) | 36 (30.5%) | 46 (28.6%) |
| ≧ 200 mg/L | 0 | 1 (4.3%) | 1 (2.3%) | 5 (9.1%) | 5 (7.9%) | 10 (8.5%) | 11 (6.8%) |

Creatinine clearance at screening (ml/min)

| | | | | | | | |
|---|---|---|---|---|---|---|---|
| Number | 85 | 85 | 170 | 254 | 255 | 509 | 679 |
| Mean (SD) | 136.52 (49.86) | 128.90 (45.17) | 132.71 (47.58) | 133.29 (46.67) | 131.23 (50.24) | 132.26 (48.46) | 132.37 (48.21) |
| Median | 119.38 | 121.92 | 120.95 | 123.22 | 122.03 | 122.50 | 122.25 |
| Min:Max | 66.9:302.7 | 70.8:366.4 | 66.9:366.4 | 43.9:350.2 | 48.0:460.5 | 43.9:460.5 | 43.9:460.5 |

Creatinine clearance categories at screening [n (%)]

| | | | | | | | |
|---|---|---|---|---|---|---|---|
| Number | 85 | 85 | 170 | 254 | 255 | 509 | 679 |
| <30 ml/min (severe | 0 | 0 | 0 | 0 | 0 | 0 | 0 |
| renal impairment) | | | | | | | |
| ≧30-<50 ml/min | 0 | 0 | 0 | 1 (0.4%) | 1 (0.4%) | 2 (0.4%) | 2 (0.3%) |
| (moderate renal | | | | | | | |
| impairment) | | | | | | | |
| ≧50-≦80 ml/min (mild | 7 (8.2%) | 6 (7.1%) | 13 (7.6%) | 21 (8.3%) | 30 (11.8%) | 51 (10.0%) | 64 (9.4%) |
| renal impairment) | | | | | | | |
| >80 ml/min (no | 78 (91.8%) | 79 (92.9%) | 157 (92.4%) | 232 (91.3%) | 224 (87.8%) | 456 (89.6%) | 613 (90.3%) |
| renal impairment) | | | | | | | |

GLP-1 = Glucagon like peptide-1.
Creatinine clearance value is derived using the equation of Cockcroft and Gault.

TABLE 5
Baseline efficacy variables - Safety population

| | Placebo | | | Lixisenatide | | | |
|---|---|---|---|---|---|---|---|
| | Morning | Evening | | Morning | Evening | | |
| | Injection | Injection | Combined | Injection | Injection | Combined | All |
| | (N = 85) | (N = 85) | (N = 170) | (N = 255) | (N = 255) | (N = 510) | (N = 680) |
| HbA1c (%) | | | | | | | |
| Number | 85 | 85 | 170 | 255 | 255 | 510 | 680 |
| Mean (SD) | 8.08 (0.86) | 8.04 (0.95) | 8.06 (0.90) | 8.04 (0.86) | 8.09 (0.91) | 8.06 (0.89) | 8.06 (0.89) |
| Median | 7.90 | 7.80 | 7.85 | 7.90 | 8.00 | 8.00 | 7.90 |
| Min:Max | 6.4:10.5 | 6.5:10.4 | 6.4:10.5 | 5.3:10.4 | 6.5:12.0 | 5.3:12.0 | 5.3:12.0 |
| Weight (kg) | | | | | | | |
| Number | 85 | 85 | 170 | 255 | 255 | 510 | 680 |
| Mean (SD) | 91.02 (21.01) | 89.27 (19.32) | 90.15 (20.14) | 90.09 (21.10) | 89.05 (20.74) | 89.57 (20.91) | 89.71 (20.70) |
| Median | 88.50 | 90.60 | 89.70 | 86.50 | 87.00 | 87.00 | 87.65 |
| Min:Max | 51.2:156.3 | 50.9:138.7 | 50.9:156.3 | 51.0:152.2 | 49.9:168.0 | 49.9:168.0 | 49.9:168.0 |
| FPG (mmol/L) | | | | | | | |
| Number | 85 | 85 | 170 | 255 | 255 | 510 | 680 |
| Mean (SD) | 9.41 (2.13) | 9.61 (2.42) | 9.51 (2.28) | 9.43 (2.15) | 9.31 (2.25) | 9.37 (2.20) | 9.40 (2.22) |
| Median | 9.20 | 9.20 | 9.20 | 9.10 | 9.00 | 9.00 | 9.10 |
| Min:Max | 5.1:15.7 | 5.7:18.8 | 5.1:18.8 | 5.1:20.0 | 3.9:17.5 | 3.9:20.0 | 3.9:20.0 |
| FPI (pmol/L) | | | | | | | |
| Number | 84 | 84 | 168 | 251 | 253 | 504 | 672 |
| Mean (SD) | 73.29 (54.12) | 78.29 (41.21) | 75.79 (48.02) | 83.68 (63.60) | 77.90 (65.46) | 80.78 (64.54) | 79.53 (60.84) |
| Median | 57.65 | 69.35 | 66.76 | 68.95 | 64.14 | 66.15 | 66.55 |
| Min:Max | 15.5:353.9 | 17.4:218.6 | 15.5:353.9 | 13.1:542.4 | 12.2:826.5 | 12.2:826.5 | 12.2:826.5 |
| 2-hour post-prandial | | | | | | | |
| plasma glucose* | | | | | | | |
| (mmol/L) | | | | | | | |
| Number | 85 | 84 | 169 | 253 | 252 | 505 | 674 |
| Mean (SD) | 15.56 (3.61) | 15.46 (3.26 ) | 15.51 (3.43) | 15.62 (3.97) | 15.46 (4.03) | 15.54 (4.00) | 15.53 (3.86) |
| Median | 15.40 | 15.30 | 15.30 | 15.40 | 15.50 | 15.40 | 15.40 |
| Min:Max | 6.7:26.7 | 9.7:27.8 | 6.7:27.8 | 5.8:28.4 | 6.9:27.9 | 5.8:28.4 | 5.8:28.4 |
| Glucose excursion* | | | | | | | |
| (mmol/L) | | | | | | | |
| Number | 84 | 84 | 168 | 252 | 252 | 504 | 672 |
| Mean (SD) | 5.92 (2.96) | 5.35 (2.64) | 5.64 (2.81) | 6.09 (3.09) | 5.80 (3.13) | 5.94 (3.11) | 5.87 (3.04) |
| Median | 5.60 | 5.80 | 5.70 | 5.90 | 5.70 | 5.80 | 5.80 |
| Min:Max | −0.6:15.0 | −5.0:10.1 | −5.0:15.0 | −5.2:16.0 | −10.1:14.2 | −10.1:16.0 | −10.1:16.0 |
| HOMA-β | | | | | | | |
| Number | 84 | 84 | 168 | 251 | 252 | 503 | 671 |
| Mean (SD) | 42.28 (53.13) | 40.68 (27.28) | 41.48 (42.12) | 42.82 (32.33) | 45.21 (46.09) | 44.02 (39.80) | 43.38 (40.38) |
| Median | 27.73 | 34.52 | 32.17 | 34.93 | 32.48 | 34.04 | 33.68 |
| Min:Max | 5.8:429.0 | 5.5:185.2 | 5.5:429.0 | 2.5:300.4 | 4.7:420.0 | 2.5:420.0 | 2.5:429.0 |

*The meal challenge test was performed on patients in the morning injection arms.
FPG = Fasting Plasma Glucose.
FPI = Fasting Plasma Insulin.
Glucose excursion = 2-hour postprandial plasma glucose - plasma glucose 30 minutes prior to the meal test before study drug administration.

6.1.4 Dosage and Duration

The average treatment exposure was similar across treatment groups: 549.9 days (78.6 weeks) in the combined placebo, 543.9 days (77.7 weeks) in the lixisenatide morning injection arm, and 515.6 days (73.7 weeks) in the lixisenatide evening injection arm (Table 6). Of the 510 lixisenatide-treated patients, 450 (90.2% for morning injection and 86.3% for evening injection) were exposed to IP for 24 weeks (169 days) or longer, and 310 (62.4% and 59.2%, respectively) were exposed for 18 months (547 days) or longer. Five patients did not record the last administration date on CRF page “End of treatment” and hence their durations of exposure were set to missing following the SAP data handling convention.

At the end of double-blind treatment, the proportion of patients who reached the target daily dose of 20 μg was lower in lixisenatide arms (91.4% for morning injection and 91.8% for evening injection), compared to the combined placebo group (97.6%) (Table 7). Similar result was observed at the end of 24-week double-blind treatment period, with 92.2% in each lixisenatide arm versus 97.1% in the combined placebo group (Table 8). The dose at the end of titration is presented in Table 30.

TABLE 6
Exposure to investigational product - Safety population

| | Placebo | | | Lixisenatide | | |
|---|---|---|---|---|---|---|
| | Morning | Evening | | Morning | Evening | |
| | Injection | Injection | Combined | Injection | Injection | Combined |
| | (N = 85) | (N = 85) | (N = 170) | (N = 255) | (N = 255) | (N = 510) |
| Cumulative duration of | 125.7 | 127.2 | 253.0 | 376.7 | 358.5 | 735.3 |
| treatment exposure | | | | | | |
| (patient years) | | | | | | |
| Duration of study treatment (days) | | | | | | |
| Number | 83 | 85 | 168 | 253 | 254 | 507 |
| Mean (SD) | 553.4 (183.4) | 546.6 (182.7) | 549.9 (182.5) | 543.9 (196.7) | 515.6 (216.6) | 529.7 (207.2) |
| Median | 568.0 | 565.0 | 567.5 | 564.0 | 561.5 | 564.0 |
| Min:Max | 9:898 | 25:821 | 9:898 | 4:875 | 7:841 | 4:875 |
| Duration of study treatment | | | | | | |
| by category [n (%)] | | | | | | |
| Missing duration | 2 (2.4%) | 0 | 2 (1.2%) | 2 (0.8%) | 1 (0.4%) | 3 (0.6%) |
| 1-14 days | 1 (1.2%) | 0 | 1 (0.6%) | 5 (2.0%) | 4 (1.6%) | 9 (1.8%) |
| 15-28 days | 1 (1.2%) | 1 (1.2%) | 2 (1.2%) | 5 (2.0%) | 3 (1.2%) | 8 (1.6%) |
| 29-56 days | 0 | 2 (2.4%) | 2 (1.2%) | 1 (0.4%) | 7 (2.7%) | 8 (1.6%) |
| 57-84 days | 1 (1.2%) | 1 (1.2%) | 2 (1.2%) | 4 (1.6%) | 8 (3.1%) | 12 (2.4%) |
| 85-168 days | 2 (2.4%) | 4 (4.7%) | 6 (3.5%) | 8 (3.1%) | 12 (4.7%) | 20 (3.9%) |
| 169-364 days | 6 (7.1%) | 4 (4.7%) | 10 (5.9%) | 17 (6.7%) | 23 (9.0%) | 40 (7.8%) |
| 365-546 days | 21 (24.7%) | 19 (22.4%) | 40 (23.5%) | 54 (21.2%) | 46 (18.0%) | 100 (19.6%) |
| 547-728 days | 43 (50.6%) | 47 (55.3%) | 90 (52.9%) | 130 (51.0%) | 129 (50.6%) | 259 (50.8%) |
| >728 days | 8 (9.4%) | 7 (8.2%) | 15 (8.8%) | 29 (11.4%) | 22 (8.6%) | 51 (10.0%) |
| Cumulative duration of | | | | | | |
| study treatment by | | | | | | |
| category [n (%)] | | | | | | |
| Missing duration | 2 (2.4%) | 0 | 2 (1.2%) | 2 (0.8%) | 1 (0.4%) | 3 (0.6%) |
| ≧1 day | 83 (97.6%) | 85 (100%) | 168 (98.8%) | 253 (99.2%) | 254 (99.6%) | 507 (99.4%) |
| ≧15 days | 82 (96.5%) | 85 (100%) | 167 (98.2%) | 248 (97.3%) | 250 (98.0%) | 498 (97.6%) |
| ≧29 days | 81 (95.3%) | 84 (98.8%) | 165 (97.1%) | 243 (95.3%) | 247 (96.9%) | 490 (96.1%) |
| ≧57 days | 81 (95.3%) | 82 (96.5%) | 163 (95.9%) | 242 (94.9%) | 240 (94.1%) | 482 (94.5%) |
| ≧85 days | 80 (94.1%) | 81 (95.3%) | 161 (94.7%) | 238 (93.3%) | 232 (91.0%) | 470 (92.2%) |
| ≧169 days | 78 (91.8%) | 77 (90.6%) | 155 (91.2%) | 230 (90.2%) | 220 (86.3%) | 450 (88.2%) |
| ≧365 days | 72 (84.7%) | 73 (85.9%) | 145 (85.3%) | 213 (83.5%) | 197 (77.3%) | 410 (80.4%) |
| ≧547 days | 51 (60.0%) | 54 (63.5%) | 105 (61.8%) | 159 (62.4%) | 151 (59.2%) | 310 (60.8%) |
| ≧729 days | 8 (9.4%) | 7 (8.2%) | 15 (8.8%) | 29 (11.4%) | 22 (8.6%) | 51 (10.0%) |

Duration of exposure = (date of the last double-blind investigational product injection − date of the first double-blind investigational product injection) + 1.

TABLE 7
Number (%) of patients by final dose at the end of the double-blind treatment - Safety population

| | Placebo | | | Lixisenatide | | |
|---|---|---|---|---|---|---|
| | Morning | Evening | | Morning | Evening | |
| | Injection | Injection | Combined | Injection | Injection | Combined |
| Final Dose | (N = 85) | (N = 85) | (N = 170) | (N = 255) | (N = 255) | (N = 510) |
| 10 μg | 1 (1.2%) | 1 (1.2%) | 2 (1.2%) | 7 (2.7%) | 9 (3.5%) | 16 (3.1%) |
| 15 μg | 2 (2.4%) | 0 | 2 (1.2%) | 14 (5.5%) | 12 (4.7%) | 26 (5.1%) |
| 20 μg | 82 (96.5%) | 84 (98.8%) | 166 (97.6%) | 233 (91.4%) | 234 (91.8%) | 467 (91.6%) |
| >20 μg | 0 | 0 | 0 | 1 (0.4%) | 0 | 1 (0.2%) |

Dose = Dose of active drug or volume-matched placebo.
Note:
Percents are calculated using the number of safety patients as the denominator.

TABLE 8
Number (%) of patients by final dose at the end of the 24-week treatment - Safety population

| | Placebo | | | Lixisenatide | | |
|---|---|---|---|---|---|---|
| | Morning | Evening | | Morning | Evening | |
| Final Dose at the | Injection | Injection | Combined | Injection | Injection | Combined |
| end of Week 24 | (N = 85) | (N = 85) | (N = 170) | (N = 255) | (N = 255) | (N = 510) |
| 10 μg | 2 (2.4%) | 1 (1.2%) | 3 (1.8%) | 6 (2.4%) | 9 (3.5%) | 15 (2.9%) |
| 15 μg | 2 (2.4%) | 0 | 2 (1.2%) | 14 (5.5%) | 11 (4.3%) | 25 (4.9%) |
| 20 μg | 81 (95.3%) | 84 (98.8%) | 165 (97.1%) | 235 (92.2%) | 235 (92.2%) | 470 (92.2%) |

Dose = Dose of active drug or volume-matched placebo.
Note:
Percents are calculated using the number of safety patients as the denominator.

6.2 Efficacy

6.2.1 Primary Efficacy Endpoint

### Main Analysis

Table 9 summarizes the results of the primary efficacy parameter, change from baseline to Week 24 (LOCF) in HbA_{1c} using an ANCOVA analysis.

According to the pre-specified primary analysis, a statistically significant reduction of HbA_{1c}, was demonstrated from baseline to Week 24 in both lixisenatide arms, compared to the combined placebo group (for lixisenatide morning injection, LS mean difference=−0.48%; p-value=<0.0001; for lixisenatide evening injection, LS mean difference=−0.37%; p-value=<0.0001).

TABLE 9
Mean change in HbA1c (%) from baseline to Week 24 - mITT population

| | Placebo | Lixisenatide | |
|---|---|---|---|
| | Combined | Morning Injection | Evening Injection |
| HbA1c (%) | (N = 170) | (N = 255) | (N = 255) |
| Baseline | | | |
| Number | 164 | 244 | 239 |
| Mean (SD) | 8.02 (0.89) | 8.07 (0.90) | 8.07 (0.89) |
| Median | 7.80 | 8.00 | 7.90 |
| Min:Max | 6.4:10.5 | 5.3:12.0 | 6.5:10.2 |
| Week 24 | | | |
| (LOCF) | | | |
| Number | 164 | 244 | 239 |
| Mean (SD) | 7.67 (1.08) | 7.24 (0.99) | 7.34 (1.04) |
| Median | 7.55 | 7.10 | 7.10 |
| Min:Max | 5.2:13.8 | 5.1:11.0 | 5.4:11.7 |
| Change from | | | |
| baseline to | | | |
| Week 24 | | | |
| (LOCF) | | | |
| Number | 164 | 244 | 239 |
| Mean (SD) | −0.35 (1.06) | −0.83 (0.91) | −0.73 (0.84) |
| Median | −0.30 | −0.90 | −0.70 |
| Min:Max | −3.6:5.8 | −3.3:2.9 | −3.0:2.5 |
| LS Mean | −0.38 (0.075) | −0.87 (0.065) | −0.75 (0.066) |
| (SE)^{(a)} | | | |
| LS Mean | | −0.48 (0.088) | −0.37 (0.088) |
| difference | | | |
| (SE) vs. | | | |
| placebo | | | |
| combined^{(a)} | | | |
| 95% CI | | (−0.657 to −0.312) | (−0.540 to −0.193) |
| p-value | | <0.0001 | <0.0001 |

^{(a)}Analysis of covariance (ANCOVA) model with treatment groups (morning injection lixisenatide and placebo arms, evening injection lixisenatide and placebo arms), randomization strata of screening HbA1c (<8.0, ≧8.0%), randomization strata of screening BMI (<30, ≧30 kg/m²), and country as fixed effects and baseline HbA1c value as a covariate.
The comparison between each lixisenatide arm and the placebo combined group was achieved through appropriate contrasts.
LOCF = Last observation carried forward.
The analysis included measurements obtained before the introduction of rescue medication and up to 3 days after the last dose of the double-blind investigational product injection on or before Visit 12 (Week 24), or Day 169 if Visit 12 (Week 24) is not available.
Patients with both baseline and Week 24 (LOCF) measurements were included.

### Secondary Analysis

Table 10 summarizes the proportion of patients with treatment response HbA_{1c}≦6.5% or ≦7% at Week 24, respectively. Treatment responses were similar in lixisenatide arms. The analysis of HbA_{1c} responders using the CMH method showed a statistically significant treatment difference between each lixisenatide arm versus the combined placebo (for HbA_{1c}≦6.5% at Week 24, p-value=0.0003 for lixisenatide morning injection and p-value=0.0120 for lixisenatide evening injection; for HbA_{1c}<7% at Week 24, p-value=<0.0001 for both lixisenatide arms).

TABLE 10
Number (%) of patients with HbA1c value ≦6.5% or <7%
at Week 24 - mITT population

| | | Lixisenatide | |
|---|---|---|---|
| | Placebo | Morning | Evening |
| | Combined | Injection | Injection |
| HbA1c (%) | (N = 170) | (N = 255) | (N = 255) |
| Number | 164 | 244 | 239 |
| ≦6.5% | 17 (10.4%) | 58 (23.8%) | 46 (19.2%) |
| >6.5% | 147 (89.6%) | 186 (76.2%) | 193 (80.8%) |
| p-value vs. placebo | — | 0.0003 | 0.0120 |
| combined^{(a)} | | | |
| Number | 164 | 244 | 239 |
| <7.0% | 36 (22.0%) | 105 (43.0%) | 97 (40.6%) |
| ≧7.0% | 128 (78.0%) | 139 (57.0%) | 142 (59.4%) |
| p-value vs. placebo | — | <0.0001 | <0.0001 |
| combined^{(a)} | | | |

^{(a)}Cochran-Mantel-Haenszel (CMH) method stratified by randomization strata of screening HbA1c (<8.0 or ≧8.0%) and randomization strata of screening body mass index (<30 or ≧30 kg/m²).
The analysis included measurements obtained before the introduction of rescue medication and up to 3 days after the last dose of the double-blind investigational product injection on or before Visit 12 (Week 24), or Day 169 if Visit 12 (Week 24) is not available.

6.2.2 Key Secondary Efficacy Endpoints

The ANCOVA analyses of 2-hour PPG, FPG, body weight, HOMA-β, FPI and glucose excursion are presented in this section. Error! Reference source not found. and Error! Reference source not found. illustrate the mean (±SE) change from baseline in FPG and body weight over time during the main 24-week double-blind treatment period. Mean (±SE) changes from baseline in FPG and body weight over time up to Week 76 are depicted in Error! Reference source not found. and Error! Reference source not found. in the appendix respectively. The percentage of patients who were rescued during the main 24 week double-blind treatment period is presented in Table 16.

A statistically significant improvement in 2-hour PPG was demonstrated in the lixisenatide morning injection arm, compared to the placebo morning injection arm with a LS mean difference of −4.51 mmol/L (p-value<0.0001) (Table 11). Treatment with lixisenatide substantially decreased glucose excursion after a standardized meal from baseline to Week 24 compared to the combined placebo group (LS mean difference=−3.88 mmol/L with a 95% CI (−4.818 to −2.939) (Table 18).

For FPG, both lixisenatide arms showed a statistically significant decrease from baseline to Week 24 compared to the combined placebo group (in the lixisenatide morning injection arm, LS mean difference=−0.94 mmol/L and p-value=<0.0001; in the lixisenatide evening injection arm, LS mean difference=−0.56 mmol/L and p-value=0.0046) (Table 12).

The LS mean decrease in body weight was 2.01 kg in the lixisenatide morning injection arm and 2.02 kg in the lixisenatide evening injection arm, compared to 1.64 kg in the combined placebo group, with no significant difference observed (Table 13). The percentage of patients who had ≧5% weight loss from baseline to Week 24 was higher in both lixisenatide arms (14.9% for morning injection and 19.3% for evening injection) than in the combined placebo group (11.3%) (Table 14).

Per the testing strategy for multiplicity adjustment, the inferential testing for the subsequent efficacy variables was exploratory, since the body weight analysis failed to show a statistically significant difference (Error! Reference source not found).

A noticeable improvement in β-cell function assessed by HOMA-β was observed in both lixisenatide arms. LS mean difference was 12.12 (p-value=0.0002 without adjustment for multiplicity) in the lixisenatide morning injection arm and 8.96 (p-value=0.0071 without adjustment for multiplicity) in the evening injection arm, compared to the combined placebo group (Table 15).

Both lixisenatide arms had substantially lower rates of patients requiring rescue therapy during the main 24-week double-blind treatment period (2.7% for morning injection and 3.9% for evening injection), compared to the combined placebo group (10.6%) (Table 16).

No clinically relevant difference in FPI was observed between each lixisenatide arm and the combined placebo group (Table 17).

TABLE 11
Mean change in 2-hour post-prandial plasma glucose (mmol/L) from
baseline to Week 24 in morning injection arms - mITT population

| | Placebo | Lixisenatide |
|---|---|---|
| 2-hour post-prandial | Morning Injection | Morning Injection |
| plasma glucose (mmol/L) | (N = 85) | (N = 255) |
| Baseline | | |
| Number | 64 | 200 |
| Mean (SD) | 15.46 (3.88) | 15.81 (4.17) |
| Median | 15.00 | 15.65 |
| Min:Max | 6.7:26.7 | 5.8:28.4 |
| Week 24 (LOCF) | | |
| Number | 64 | 200 |
| Mean (SD) | 14.24 (4.06) | 10.14 (4.17) |
| Median | 14.05 | 9.40 |
| Min:Max | 6.1:23.3 | 4.1:23.7 |
| Change from baseline to | | |
| Week 24 (LOCF) | | |
| Number | 64 | 200 |
| Mean (SD) | −1.22 (4.12) | −5.67 (5.16) |
| Median | −0.90 | −5.55 |
| Min:Max | −11.2:6.6 | −22.2:11.8 |
| LS Mean (SE)^{(a)} | −1.41 (0.588) | −5.92 (0.415) |
| LS Mean difference (SE) vs. | | −4.51 (0.579) |
| placebo morning injection^{(a)} | | |
| 95% CI | | (−5.652 to −3.371) |
| p-value | | <0.0001 |

^{(a)}Analysis of covariance (ANCOVA) with treatment groups (morning injection lixisenatide and placebo arms), randomization strata of screening HbA1c (<8.0, ≧8.0%), randomization strata of screening body mass index (<30, ≧30 kg/m²), and country as fixed effects and 2-hour post-prandial plasma glucose value at baseline as a covariate.
LOCF = Last observation carried forward.
The analysis included measurements obtained before the introduction of rescue medication and up to the date of the last dose of the double-blind investigational product injection on or before Visit 12 (Week 24), or Day 169 if Visit 12 (Week 24) is not available.
Patients with both baseline and Week 24 (LOCF) measurements were included.

TABLE 12
Mean change in fasting plasma glucose (mmol/L) from baseline to Week 24 - mITT population

| | | Lixisenatide | |
|---|---|---|---|
| | Placebo Combined | Morning Injection | Evening Injection |
| Fasting plasma glucose (mmol/L) | (N = 170) | (N = 255) | (N = 255) |
| Baseline | | | |
| Number | 170 | 253 | 255 |
| Mean (SD) | 9.51 (2.28) | 9.46 (2.21) | 9.28 (2.19) |
| Median | 9.20 | 9.20 | 9.00 |
| Min:Max | 5.1:18.8 | 5.1:20.0 | 3.9:17.5 |
| Week 24 (LOCF) | | | |
| Number | 170 | 253 | 255 |
| Mean (SD) | 9.24 (2.48) | 8.26 (2.10) | 8.51 (2.23) |
| Median | 8.80 | 7.85 | 8.20 |
| Min:Max | 5.3:18.4 | 4.8:17.2 | 4.5:19.4 |
| Change from baseline to Week 24 | | | |
| (LOCF) | | | |
| Number | 170 | 253 | 255 |
| Mean (SD) | −0.27 (2.42) | −1.20 (2.40) | −0.77 (2.02) |
| Median | −0.30 | −1.10 | −0.90 |
| Min:Max | −8.8:10.4 | −10.3:7.0 | −7.7:7.5 |
| LS Mean (SE)^{(a)} | −0.25 (0.166) | −1.19 (0.145) | −0.81 (0.146) |
| LS Mean difference (SE) vs. | | −0.94 (0.196) | −0.56 (0.196) |
| placebo combined^{(a)} | | | |
| 95% CI | | (−1.329 to −0.559) | (−0.944 to −0.173) |
| p-value | | <0.0001 | 0.0046 |

^{(a)}Analysis of covariance (ANCOVA) with treatment groups (morning injection lixisenatide and placebo arms, evening injection lixisenatide and placebo arms), randomization strata of screening HbA1c (<8.0, ≧8.0%), randomization strata of screening body mass index (<30, ≧30 kg/m²), and country as fixed effects and fasting plasma glucose value at baseline as a covariate.
The comparison between each lixisenatide arm and the placebo combined group was achieved through appropriate contrasts.
LOCF = Last observation carried forward.
The analysis included measurements obtained before the introduction of rescue medication and up to 1 day after the date of the last dose of the double-blind investigational product injection on or before Visit 12 (Week 24), or Day 169 if Visit 12 (Week 24) is not available.
Patients with both baseline and Week 24 (LOCF) measurements were included.

TABLE 13
Mean change in body weight (kg) from baseline to
Week 24 - mITT population

| | | Lixisenatide | |
|---|---|---|---|
| | Placebo | Morning | |
| | Combined | Injection | Evening Injection |
| Body weight (kg) | (N = 170) | (N = 255) | (N = 255) |
| Baseline | | | |
| Number | 168 | 248 | 249 |
| Mean (SD) | 90.40 (20.12) | 90.14 (21.04) | 89.01 (20.72) |
| Median | 90.05 | 86.75 | 87.00 |
| Min:Max | 50.9:156.3 | 51.0:152.2 | 49.9:168.0 |
| Week 24 (LOCF) | | | |
| Number | 168 | 248 | 249 |
| Mean (SD) | 88.60 (19.45) | 88.07 (20.79) | 86.85 (19.84) |
| Median | 87.50 | 85.08 | 85.00 |
| Min:Max | 50.0:145.9 | 49.4:151.9 | 48.8:168.0 |
| Change from | | | |
| baseline to | | | |
| Week 24 (LOCF) | | | |
| Number | 168 | 248 | 249 |
| Mean (SD) | −1.80 (3.14) | −2.08 (3.11) | −2.16 (3.49) |
| Median | −1.65 | −1.70 | −1.50 |
| Min:Max | −15.5:6.0 | −18.0:7.0 | −16.5:11.5 |
| LS Mean (SE)^{(a)} | −1.64 (0.269) | −2.01 (0.234) | −2.02 (0.236) |
| LS Mean | | −0.38 (0.314) | −0.39 (0.315) |
| difference (SE) | | | |
| vs. placebo | | | |
| combined^{(a)} | | | |
| 95% CI | | (−0.995 to 0.239) | (−1.006 to 0.230) |
| p-value | | 0.2293 | 0.2181 |

^{(a)}Analysis of covariance (ANCOVA) with treatment groups (morning injection lixisenatide and placebo arms, evening injection lixisenatide and placebo arms), randomization strata of screening HbA1c (<8.0, ≧8.0%), randomization strata of screening body mass index (<30, ≧30 kg/m²), and country as fixed effects and body weight at baseline as a covariate.
The comparison between each lixisenatide arm and the placebo combined group was achieved through appropriate contrasts.
LOCF = Last observation carried forward.
The analysis included measurements obtained before the introduction of rescue medication and up to 3 days after the date of the last dose of the double-blind investigational product injection on or before Visit 12 (Week 24), or Day 169 if Visit 12 (Week 24) is not available.
Patients with both baseline and Week 24 (LOCF) measurements were included.

TABLE 14
Number (%) of patients with >=5% weight loss from
baseline to Week 24 - mITT population

| | | Lixisenatide | |
|---|---|---|---|
| | Placebo Combined | Morning Injection | Evening Injection |
| Weight loss | (N = 170) | (N = 255) | (N = 255) |
| Number | 168 | 248 | 249 |
| ≧5% | 19 (11.3%) | 37 (14.9%) | 48 (19.3%) |
| <5% | 149 (88.7%) | 211 (85.1%) | 201 (80.7%) |

The analysis included measurements obtained before the introduction of rescue medication and up to 3 days after the last dose of the double-blind investigational product injection on or before Visit 12 (Week 24), or Day 169 if Visit 12 (Week 24) is not available.

TABLE 15
Mean change in HOMA-β from baseline to Week 24 - mITT population

| | Placebo | Lixisenatide | |
|---|---|---|---|
| | Combined | Morning Injection | Evening Injection |
| HOMA-β | (N = 170) | (N = 255) | (N = 255) |
| Baseline | | | |
| Number | 157 | 235 | 226 |
| Mean (SD) | 41.66 (42.91) | 42.97 (33.15) | 45.45 (42.76) |
| Median | 32.34 | 34.92 | 33.56 |
| Min:Max | 5.5:429.0 | 2.5:300.4 | 4.7:420.0 |
| Week 24 (LOCF) | | | |
| Number | 157 | 235 | 226 |
| Mean (SD) | 39.82 (29.67) | 52.64 (44.56) | 52.19 (41.83) |
| Median | 34.60 | 41.96 | 39.63 |
| Min:Max | 4.8:221.6 | 7.2:352.6 | 6.1:335.4 |
| Change from | | | |
| baseline to | | | |
| Week 24 (LOCF) | | | |
| Number | 157 | 235 | 226 |
| Mean (SD) | −1.85 (31.57) | 9.67 (36.36) | 6.75 (36.56) |
| Median | 1.64 | 5.31 | 6.01 |
| Min:Max | −307.8:76.6 | −179.3:246.7 | −368.0:178.4 |
| LS Mean (SE)^{(a)} | −4.16 (2.823) | 7.96 (2.450) | 4.80 (2.486) |
| LS Mean | | 12.12 (3.278) | 8.96 (3.317) |
| difference (SE) | | | |
| vs. placebo | | | |
| combined^{(a)} | | | |
| 95% CI | | (5.685 to 18.559) | (2.450 to 15.477) |
| p-value | | 0.0002 | 0.0071 |

^{(a)}Analysis of covariance (ANCOVA) model with treatment groups (morning injection lixisenatide and placebo arms, evening injection lixisenatide and placebo arms), randomization strata of screening HbA1c (<8.0, ≧8.0%), randomization strata of screening body mass index (<30, ≧30 kg/m²), and country as fixed effects and baseline HOMA-β value as a covariate.
The comparison between each lixisenatide arm and the placebo combined group was achieved through appropriate contrasts.
LOCF = Last observation carried forward.
The analysis included measurements obtained before the introduction of rescue medication and up to 1 day after the last dose of the double-blind investigational product injection on or before Visit 12 (Week 24), or Day 169 if Visit 12 (Week 24) is not available.
Patients with both baseline and Week 24 (LOCF) measurements were included.

TABLE 16
Number (%) of patients requiring rescue therapy during the
main 24-week double-blind treatment period - mITT population

| | Placebo | Lixisenatide | |
|---|---|---|---|
| Requiring | Combined | Morning Injection | Evening Injection |
| rescue therapy | (N = 170) | (N = 255) | (N = 255) |
| Number | 170 | 255 | 255 |
| Yes | 18 (10.6%) | 7 (2.7%) | 10 (3.9%) |
| No | 152 (89.4%) | 248 (97.3%) | 245 (96.1%) |
| p-value vs. placebo | — | 0.0007 | 0.0063 |
| combined^{(a)} | | | |

^{(a)}Cochran-Mantel-Haenszel (CMH) method stratified by randomization strata of screening HbA1c (<8.0 or ≧8.0%) and randomization strata of screening body mass index (<30 or ≧30 kg/m²).

TABLE 17
Mean change in fasting plasma insulin (pmol/L) from baseline
to Week 24 - mITT population

| Fasting | Placebo | Lixisenatide | |
|---|---|---|---|
| plasma insulin | Combined | Morning Injection | Evening Injection |
| (pmol/L) | (N = 170) | (N = 255) | (N = 255) |
| Baseline | | | |
| Number | 157 | 237 | 229 |
| Mean (SD) | 75.51 (48.19) | 84.41 (64.97) | 75.92 (46.05) |
| Median | 66.73 | 69.02 | 64.22 |
| Min:Max | 16.8:353.9 | 13.1:542.4 | 12.3:267.6 |
| Week 24 | | | |
| (LOCF) | | | |
| Number | 157 | 237 | 229 |
| Mean (SD) | 72.18 (45.40) | 78.51 (58.38) | 76.36 (46.36) |
| Median | 60.99 | 65.94 | 64.29 |
| Min:Max | 13.3:282.9 | 11.3:518.6 | 10.2:279.0 |
| Change from | | | |
| baseline to | | | |
| Week 24 | | | |
| (LOCF) | | | |
| Number | 157 | 237 | 229 |
| Mean (SD) | −3.33 (37.59) | −5.89 (46.86) | 0.44 (36.72) |
| Median | −0.14 | −3.37 | 0.36 |
| Min:Max | −214.9:159.8 | −351.3:233.8 | −151.3:251.1 |
| LS Mean | −6.23 (3.254) | −5.09 (2.812) | −1.88 (2.862) |
| (SE)^{(a)} | | | |
| LS Mean | | 1.14 (3.777) | 4.35 (3.805) |
| difference (SE) | | | |
| vs. placebo | | | |
| combined^{(a)} | | | |
| 95% CI | | (−6.275 to 8.561) | (−3.121 to 11.826) |
| p-value | | 0.7622 | 0.2532 |

^{(a)}Analysis of covariance (ANCOVA) with treatment groups (morning injection lixisenatide and placebo arms, evening injection lixisenatide and placebo arms), randomization strata of screening HbA1c (<8.0, ≧8.0%), randomization strata of screening body mass index (<30, ≧30 kg/m²), and country as fixed effects and fasting plasma insulin value at baseline as a covariate.
The comparison between each lixisenatide arm and the placebo combined group was achieved through appropriate contrasts.
LOCF = Last observation carried forward.
The analysis included measurements obtained before the introduction of rescue medication and up to 1 day after the date of the last dose of the double-blind investigational product injection on or before Visit 12 (Week 24), or Day 169 if Visit 12 (Week 24) is not available.
Patients with both baseline and Week 24 (LOCF) measurements were included.

TABLE 18
Mean change in glucose excursion (mmol/L) from baseline
to week 24 in morning injection arms - mITT population

| | Placebo | Lixisenatide |
|---|---|---|
| | Morning Injection | Morning Injection |
| Glucose excursion (mmol/L) | (N = 85) | (N = 255) |
| Baseline | | |
| Number | 63 | 198 |
| Mean (SD) | 5.80 (2.95) | 6.17 (3.20) |
| Median | 5.50 | 6.10 |
| Min:Max | −0.6:15.0 | −5.2:16.0 |
| Week 24 (LOCF) | | |
| Number | 63 | 198 |
| Mean (SD) | 5.24 (2.73) | 1.85 (3.48) |
| Median | 5.50 | 1.32 |
| Min:Max | −0.7:11.0 | −5.2:11.1 |
| Change from baseline | | |
| to Week 24 (LOCF) | | |
| Number | 63 | 198 |
| Mean (SD) | −0.56 (2.85) | −4.32 (4.46) |
| Median | −0.20 | −4.15 |
| Min:Max | −11.1:4.4 | −18.6:9.4 |
| LS Mean (SE)^{(a)} | −0.76 (0.483) | −4.64 (0.340) |
| LS Mean difference (SE) vs. | | −3.88 (0.477) |
| placebo morning injection^{(a)} | | |
| 95% CI | | (−4.818 to −2.939) |

^{(a)}Analysis of covariance (ANCOVA) model with treatment groups (morning injection lixisenatide and placebo arms), randomization strata of screening HbA1c (<8.0, ≧8.0%), randomization strata of screening body mass index (<30, ≧30 kg/m²), and country as fixed effects and baseline glucose excursion value as a covariate.
LOCF = Last observation carried forward.
Glucose excursion = 2-hour postprandial plasma glucose − plasma glucose 30 minutes prior to the meal test before study drug administration.
The analysis included measurements obtained before the introduction of rescue medication and up to the date of the last dose of the double-blind investigational product injection on or before Visit 12 (Week 24), or Day 169 if Visit 12 (Week 24) is not available.
Patients with both baseline and Week 24 (LOCF) measurements were included.

6.3 Safety

An overview of the adverse events observed during the on-treatment period of the whole study is provided in Table 19. The proportion of patients who experienced TEAEs was higher in the lixisenatide-treated patients (84.7% for morning injection and 83.5% for evening injection), compared to the combined placebo group (75.3%). One patient in the lixisenatide evening arm had a TEAE of pancreatic carcinoma leading to death. Two patients in the lixisenatide evening arm died due to post-treatment AEs (haemothorax and lymphoma respectively). The lixisenatide evening injection arm had higher rate of serious TEAEs (10.2%), followed by the lixisenatide morning injection arm (8.2%) and the combined placebo group (6.5%). Similar pattern was also observed in TEAEs leading to treatment discontinuation with 9.4% in the lixisenatide evening injection arm, 8.2% in the lixisenatide morning injection arm, compared to 3.5% in the combined placebo group. Table 20, Table 21, and Table 22 summarize TEAEs leading to death, serious TEAEs, and TEAEs leading to treatment discontinuation by primary SOC, HLGT, HLT and PT, respectively. The most common TEAE leading to treatment discontinuation was nausea in both lixisenatide-treated groups (6 [2.4%] patients for morning injection and 7 [2.7%] for evening injection), while no patients discontinued treatment due to nausea in the combined placebo group.

Table 32 in the appendix presents the incidences of TEAEs during the on-treatment period of the whole study occurring in at least 1% of patients in the combined placebo group or any individual lixisenatide group. Nausea was the most frequently reported TEAE in both lixisenatide-treated groups (64 [25.1%] patients for morning injection and 63 [24.7%] for evening injection). Sixteen placebo-treated patients (9.4%) reported nausea. The second most frequently reported TEAE in the lixisenatide-treated patients was headache (49 [19.2%] patients for morning injection and 42 [16.5%] for evening injection), followed by diarrhoea (39 [15.3%] patients for morning injection and 36 [14.1%] for evening injection) and vomiting (35 [13.7%] patients for morning injection and 40 [15.7%] for evening injection). In the combined placebo group, 28 [16.5%] patients reported headache, 20 [11.8%] diarrhoea, and 9 [5.3%] vomiting.
Patient #276407001 (lixisenatide morning injection), a 73-year-old male, with a history of left intracranial aneurysm, benign prostatic hyperplasia, atrial fibrillation and haemorroids, developed icterus on 15 Jan. 2010 (32 weeks after the first administration of study drug). He did not feel well and had familial problems, leading him to consider stopping participation in the study. On 12 Feb. 2010, he was found to be icteric with high levels of tumor marker leading to suspect a pancreas cancer and was hospitalized. No corrective treatment was given, and study medication was permanently discontinued on 12 Feb. 2010. A CT examination confirmed the high suspicion of pancreatic carcinoma. On 24 Feb. 2010, surgery for malignant tumor of the pancreas head was performed. The patient started chemotherapy on 12 Apr. 2010, as an out-patient. The patient, tried to commit suicide with tablets on 23 May 2010. Chemotherapy (10 cycles until July 2010) was unsuccessful and stopped. Early October 2010, the patient experienced intestinal obstruction, and underwent surgery. After being transferred to a hospice, the patient died of malignancy on 21 Nov. 2010. Death was clinically expected, no resuscitation was attempted and no autopsy was performed. Per investigator, the causal assessment was “not associated”. The Sponsor's causal assessment was “excluded”.

TABLE 19
Overview of adverse event profile: treatment emergent adverse events during the on-treatment period of the whole study-
Safety population

| | Placebo | | | Lixisenatide | | |
|---|---|---|---|---|---|---|
| | Morning | Evening | | Morning | Evening | |
| | Injection | Injection | Combined | Injection | Injection | Combined |
| | (N = 85) | (N = 85) | (N = 170) | (N = 255) | (N = 255) | (N = 510) |
| Patients with any TEAE | 60 (70.6%) | 68 (80.0%) | 128 (75.3%) | 216 (84.7%) | 213 (83.5%) | 429 (84.1%) |
| Patients with any serious TEAE | 2 (2.4%) | 9 (10.6%) | 11 (6.5%) | 21 (8.2%) | 26 (10.2%) | 47 (9.2%) |
| Patients with any TEAE leading to death | 0 | 0 | 0 | 1 (0.4%) | 0 | 1 (0.2%) |
| Patients with any TEAE leading to permanent | 3 (3.5%) | 3 (3.5%) | 6 (3.5%) | 21 (8.2%) | 24 (9.4%) | 45 (8.8%) |
| treatment discontinuation | | | | | | |

TEAE: Treatment emergent adverse event.
On-treatment period of the whole study = the time from the first dose of double-blind study medication up to 3 days after the last dose administration.
n (%) = number and percentage of patients with at least one adverse event.

TABLE 20
Number (%) of patients experiencing TEAE(s) leading to death by primary SOC, HLGT, HLT, and PT
during the on-treatment period of the whole study - Safety population

| PRIMARY SYSTEM ORGAN CLASS | Placebo | | | Lixisenatide | | |
|---|---|---|---|---|---|---|
| HLGT: High Level Group Term | Morning | Evening | | Morning | Evening | |
| HLT: High Level Term | Injection | Injection | Combined | Injection | Injection | Combined |
| Preferred Term | (N = 85) | (N = 85) | (N = 170) | (N = 255) | (N = 255) | (N = 510) |
| Any class | 0 | 0 | 0 | 1 (0.4%) | 0 | 1 (0.2%) |
| NEOPLASMS BENIGN, MALIGNANT AND | 0 | 0 | 0 | 1 (0.4%) | 0 | 1 (0.2%) |
| UNSPECIFIED (INCL CYSTS AND POLYPS) | | | | | | |
| HLGT: Gastrointestinal neoplasms malignant and | 0 | 0 | 0 | 1 (0.4%) | 0 | 1 (0.2%) |
| unspecified | | | | | | |
| HLT: Pancreatic neoplasms malignant (excl | 0 | 0 | 0 | 1 (0.4%) | 0 | 1 (0.2%) |
| islet cell and carcinoid) | | | | | | |
| Pancreatic carcinoma | 0 | 0 | 0 | 1 (0.4%) | 0 | 1 (0.2%) |

TEAE: Treatment emergent adverse event,
SOC: System Organ Class,
HLGT: High Level Group Term,
HLT: High Level Term,
PT: Preferred Term.
On-treatment period of the whole study = the time from the first dose of double-blind study medication up to 3 days after the last dose administration.
MedDRA version: 13.1.
n (%) = number and percentage of patients with at least one TEAE leading to death.
Note:
Table sorted by SOC internationally agreed order and HLGT, HLT, PT alphabetic order.

TABLE 21
Number (%) of patients experiencing serious TEAE presented by primary SOC, HLGT, HLT, and PT during the on-treatment
period of the whole study - Safety population

| PRIMARY SYSTEM ORGAN CLASS | Placebo | | | | | | Lixisenatide | | | | | |
|---|---|---|---|---|---|---|---|---|---|---|---|---|
| HLGT: High Level Group Term | Morning | | Evening | | | | Morning | | Evening | | | |
| HLT: High Level Term | Injection | | Injection | | Combined | | Injection | | Injection | | Combined | |
| Preferred Term | (N = 85) | | (N = 85) | | (N = 170) | | (N = 255) | | (N = 255) | | (N = 510) | |
| Any class | 2 | (2.4%) | 9 | (10.6%) | 11 | (6.5%) | 21 | (8.2%) | 26 | (10.2%) | 47 | (9.2%) |
| INFECTIONS AND INFESTATIONS | 0 | | 2 | (2.4%) | 2 | (1.2%) | 2 | (0.8%) | 4 | (1.6%) | 6 | (1.2%) |
| HLGT: Bacterial infectious disorders | 0 | | 1 | (1.2%) | 1 | (0.6%) | 0 | | 1 | (0.4%) | 1 | (0.2%) |
| HLT: Escherichia infections | 0 | | 1 | (1.2%) | 1 | (0.6%) | 0 | | 0 | | 0 | |
| Escherichia urinary tract infection | 0 | | 1 | (1.2%) | 1 | (0.6%) | 0 | | 0 | | 0 | |
| HLT: Staphylococcal infections | 0 | | 0 | | 0 | | 0 | | 1 | (0.4%) | 1 | (0.2%) |
| Staphylococcal sepsis | 0 | | 0 | | 0 | | 0 | | 1 | (0.4%) | 1 | (0.2%) |
| HLGT: Infections - pathogen unspecified | 0 | | 1 | (1.2%) | 1 | (0.6%) | 1 | (0.4%) | 3 | (1.2%) | 4 | (0.8%) |
| HLT: Abdominal and gastrointestinal | 0 | | 0 | | 0 | | 0 | | 1 | (0.4%) | 1 | (0.2%) |
| infections | | | | | | | | | | | | |
| Anal abscess | 0 | | 0 | | 0 | | 0 | | 1 | (0.4%) | 1 | (0.2%) |
| HLT: Lower respiratory tract and lung | 0 | | 1 | (1.2%) | 1 | (0.6%) | 1 | (0.4%) | 2 | (0.8%) | 3 | (0.6%) |
| infections | | | | | | | | | | | | |
| Pneumonia | 0 | | 1 | (1.2%) | 1 | (0.6%) | 1 | (0.4%) | 2 | (0.8%) | 3 | (0.6%) |
| HLT: Sepsis, bacteraemia, viraemia and | 0 | | 1 | (1.2%) | 1 | (0.6%) | 0 | | 0 | | 0 | |
| fungaemia NEC | | | | | | | | | | | | |
| Bacterial sepsis | 0 | | 1 | (1.2%) | 1 | (0.6%) | 0 | | 0 | | 0 | |
| HLGT: Viral infectious disorders | 0 | | 0 | | 0 | | 1 | (0.4%) | 0 | | 1 | (0.2%) |
| HLT: Influenza viral infections | 0 | | 0 | | 0 | | 1 | (0.4%) | 0 | | 1 | (0.2%) |
| Influenza | 0 | | 0 | | 0 | | 1 | (0.4%) | 0 | | 1 | (0.2%) |
| NEOPLASMS BENIGN, MALIGNANT AND | 1 | (1.2%) | 1 | (1.2%) | 2 | (1.2%) | 3 | (1.2%) | 0 | | 3 | (0.6%) |
| UNSPECIFIED (INCL CYSTS AND POLYPS) | | | | | | | | | | | | |
| HLGT: Endocrine neoplasms malignant and | 0 | | 0 | | 0 | | 1 | (0.4%) | 0 | | 1 | (0.2%) |
| unspecified | | | | | | | | | | | | |
| HLT: Endocrine neoplasms malignant and | 0 | | 0 | | 0 | | 1 | (0.4%) | 0 | | 1 | (0.2%) |
| unspecified NEC | | | | | | | | | | | | |
| Thyroid neoplasm | 0 | | 0 | | 0 | | 1 | (0.4%) | 0 | | 1 | (0.2%) |
| HLGT: Gastrointestinal neoplasms malignant and | 0 | | 1 | (1.2%) | 1 | (0.6%) | 1 | (0.4%) | 0 | | 1 | (0.2%) |
| unspecified | | | | | | | | | | | | |
| HLT: Pancreatic neoplasms malignant (excl | 0 | | 0 | | 0 | | 1 | (0.4%) | 0 | | 1 | (0.2%) |
| islet cell and carcinoid) | | | | | | | | | | | | |
| Pancreatic carcinoma | 0 | | 0 | | 0 | | 1 | (0.4%) | 0 | | 1 | (0.2%) |
| HLT: Rectal neoplasms malignant | 0 | | 1 | (1.2%) | 1 | (0.6%) | 0 | | 0 | | 0 | |
| Rectal cancer | 0 | | 1 | (1.2%) | 1 | (0.6%) | 0 | | 0 | | 0 | |
| HLGT: Miscellaneous and site unspecified | 1 | (1.2%) | 0 | | 1 | (0.6%) | 0 | | 0 | | 0 | |
| neoplasms malignant and unspecified | | | | | | | | | | | | |
| HLT: Neoplasms malignant site unspecified | 1 | (1.2%) | 0 | | 1 | (0.6%) | 0 | | 0 | | 0 | |
| NEC | | | | | | | | | | | | |
| Signet-ring cell carcinoma | 1 | (1.2%) | 0 | | 1 | (0.6%) | 0 | | 0 | | 0 | |
| HLGT: Renal and urinary tract neoplasms | 0 | | 0 | | 0 | | 1 | (0.4%) | 0 | | 1 | (0.2%) |
| malignant and unspecified | | | | | | | | | | | | |
| HLT: Renal neoplasms malignant | 0 | | 0 | | 0 | | 1 | (0.4%) | 0 | | 1 | (0.2%) |
| Renal cell carcinoma | 0 | | 0 | | 0 | | 1 | (0.4%) | 0 | | 1 | (0.2%) |
| HLGT: Reproductive neoplasms male malignant | 0 | | 0 | | 0 | | 1 | (0.4%) | 0 | | 1 | (0.2%) |
| and unspecified | | | | | | | | | | | | |
| HLT: Prostatic neoplasms malignant | 0 | | 0 | | 0 | | 1 | (0.4%) | 0 | | 1 | (0.2%) |
| Prostate cancer | 0 | | 0 | | 0 | | 1 | (0.4%) | 0 | | 1 | (0.2%) |
| BLOOD AND LYMPHATIC SYSTEM | 0 | | 0 | | 0 | | 0 | | 1 | (0.4%) | 1 | (0.2%) |
| DISORDERS | | | | | | | | | | | | |
| HLGT: Spleen, lymphatic and reticuloendothelial | 0 | | 0 | | 0 | | 0 | | 1 | (0.4%) | 1 | (0.2%) |
| system disorders | | | | | | | | | | | | |
| HLT: Lymphatic system disorders NEC | 0 | | 0 | | 0 | | 0 | | 1 | (0.4%) | 1 | (0.2%) |
| Lymphadenitis | 0 | | 0 | | 0 | | 0 | | 1 | (0.4%) | 1 | (0.2%) |
| ENDOCRINE DISORDERS | 1 | (1.2%) | 0 | | 1 | (0.6%) | 0 | | 0 | | 0 | |
| HLGT: Thyroid gland disorders | 1 | (1.2%) | 0 | | 1 | (0.6%) | 0 | | 0 | | 0 | |
| HLT: Thyroid hypofunction disorders | 1 | (1.2%) | 0 | | 1 | (0.6%) | 0 | | 0 | | 0 | |
| Hypothyroidism | 1 | (1.2%) | 0 | | 1 | (0.6%) | 0 | | 0 | | 0 | |
| METABOLISM AND NUTRITION DISORDERS | 0 | | 0 | | 0 | | 0 | | 1 | (0.4%) | 1 | (0.2%) |
| HLGT: Appetite and general nutritional disorders | 0 | | 0 | | 0 | | 0 | | 1 | (0.4%) | 1 | (0.2%) |
| HLT: General nutritional disorders NEC | 0 | | 0 | | 0 | | 0 | | 1 | (0.4%) | 1 | (0.2%) |
| Obesity | 0 | | 0 | | 0 | | 0 | | 1 | (0.4%) | 1 | (0.2%) |
| PSYCHIATRIC DISORDERS | 0 | | 0 | | 0 | | 2 | (0.8%) | 1 | (0.4%) | 3 | (0.6%) |
| HLGT: Sleep disorders and disturbances | 0 | | 0 | | 0 | | 0 | | 1 | (0.4%) | 1 | (0.2%) |
| HLT: Disturbances in initiating and | 0 | | 0 | | 0 | | 0 | | 1 | (0.4%) | 1 | (0.2%) |
| maintaining sleep | | | | | | | | | | | | |
| Insomnia | 0 | | 0 | | 0 | | 0 | | 1 | (0.4%) | 1 | (0.2%) |
| HLGT: Somatoform and factitious disorders | 0 | | 0 | | 0 | | 1 | (0.4%) | 0 | | 1 | (0.2%) |
| HLT: Somatoform disorders | 0 | | 0 | | 0 | | 1 | (0.4%) | 0 | | 1 | (0.2%) |
| Psychosomatic disease | 0 | | 0 | | 0 | | 1 | (0.4%) | 0 | | 1 | (0.2%) |
| HLGT: Suicidal and self-injurious behaviours | 0 | | 0 | | 0 | | 1 | (0.4%) | 0 | | 1 | (0.2%) |
| NEC | | | | | | | | | | | | |
| HLT: Suicidal and self-injurious behaviour | 0 | | 0 | | 0 | | 1 | (0.4%) | 0 | | 1 | (0.2%) |
| Suicide attempt | 0 | | 0 | | 0 | | 1 | (0.4%) | 0 | | 1 | (0.2%) |
| NERVOUS SYSTEM DISORDERS | 0 | | 2 | (2.4%) | 2 | (1.2%) | 3 | (1.2%) | 2 | (0.8%) | 5 | (1.0%) |
| HLGT: Central nervous system vascular disorders | 0 | | 2 | (2.4%) | 2 | (1.2%) | 1 | (0.4%) | 1 | (0.4%) | 2 | (0.4%) |
| HLT: Central nervous system haemorrhages | 0 | | 2 | (2.4%) | 2 | (1.2%) | 1 | (0.4%) | 1 | (0.4%) | 2 | (0.4%) |
| and cerebrovascular accidents | | | | | | | | | | | | |
| Cerebral infarction | 0 | | 1 | (1.2%) | 1 | (0.6%) | 0 | | 0 | | 0 | |
| Lacunar infarction | 0 | | 1 | (1.2%) | 1 | (0.6%) | 0 | | 1 | (0.4%) | 1 | (0.2%) |
| Ruptured cerebral aneurysm | 0 | | 0 | | 0 | | 1 | (0.4%) | 0 | | 1 | (0.2%) |
| HLGT: Encephalopathies | 0 | | 0 | | 0 | | 1 | (0.4%) | 0 | | 1 | (0.2%) |
| HLT: Encephalopathies NEC | 0 | | 0 | | 0 | | 1 | (0.4%) | 0 | | 1 | (0.2%) |
| Hypertensive encephalopathy | 0 | | 0 | | 0 | | 1 | (0.4%) | 0 | | 1 | (0.2%) |
| HLGT: Neurological disorders NEC | 0 | | 0 | | 0 | | 1 | (0.4%) | 0 | | 1 | (0.2%) |
| HLT: Neurological signs and symptoms NEC | 0 | | 0 | | 0 | | 1 | (0.4%) | 0 | | 1 | (0.2%) |
| Dizziness | 0 | | 0 | | 0 | | 1 | (0.4%) | 0 | | 1 | (0.2%) |
| HLGT: Peripheral neuropathies | 0 | | 0 | | 0 | | 0 | | 1 | (0.4%) | 1 | (0.2%) |
| HLT: Mononeuropathies | 0 | | 0 | | 0 | | 0 | | 1 | (0.4%) | 1 | (0.2%) |
| Carpal tunnel syndrome | 0 | | 0 | | 0 | | 0 | | 1 | (0.4%) | 1 | (0.2%) |
| EYE DISORDERS | 0 | | 0 | | 0 | | 1 | (0.4%) | 0 | | 1 | (0.2%) |
| HLGT: Retina, choroid and vitreous | 0 | | 0 | | 0 | | 1 | (0.4%) | 0 | | 1 | (0.2%) |
| haemorrhages and vascular disorders | | | | | | | | | | | | |
| HLT: Choroid and vitreous haemorrhages | 0 | | 0 | | 0 | | 1 | (0.4%) | 0 | | 1 | (0.2%) |
| and vascular disorders | | | | | | | | | | | | |
| Vitreous haemorrhage | 0 | | 0 | | 0 | | 1 | (0.4%) | 0 | | 1 | (0.2%) |
| HLT: Retinal bleeding and vascular disorders | 0 | | 0 | | 0 | | 1 | (0.4%) | 0 | | 1 | (0.2%) |
| (excl retinopathy) | | | | | | | | | | | | |
| Retinal haemorrhage | 0 | | 0 | | 0 | | 1 | (0.4%) | 0 | | 1 | (0.2%) |
| CARDIAC DISORDERS | 0 | | 2 | (2.4%) | 2 | (1.2%) | 3 | (1.2%) | 4 | (1.6%) | 7 | (1.4%) |
| HLGT: Cardiac arrhythmias | 0 | | 1 | (1.2%) | 1 | (0.6%) | 1 | (0.4%) | 1 | (0.4%) | 2 | (0.4%) |
| HLT: Cardiac conduction disorders | 0 | | 1 | (1.2%) | 1 | (0.6%) | 0 | | 0 | | 0 | |
| Atrioventricular block first degree | 0 | | 1 | (1.2%) | 1 | (0.6%) | 0 | | 0 | | 0 | |
| HLT: Supraventricular arrhythmias | 0 | | 0 | | 0 | | 1 | (0.4%) | 0 | | 1 | (0.2%) |
| Atrial fibrillation | 0 | | 0 | | 0 | | 1 | (0.4%) | 0 | | 1 | (0.2%) |
| HLT: Ventricular arrhythmias and cardiac | 0 | | 0 | | 0 | | 0 | | 1 | (0.4%) | 1 | (0.2%) |
| arrest | | | | | | | | | | | | |
| Ventricular extrasystoles | 0 | | 0 | | 0 | | 0 | | 1 | (0.4%) | 1 | (0.2%) |
| HLGT: Coronary artery disorders | 0 | | 1 | (1.2%) | 1 | (0.6%) | 3 | (1.2%) | 2 | (0.8%) | 5 | (1.0%) |
| HLT: Coronary artery disorders NEC | 0 | | 0 | | 0 | | 1 | (0.4%) | 0 | | 1 | (0.2%) |
| Coronary artery disease | 0 | | 0 | | 0 | | 1 | (0.4%) | 0 | | 1 | (0.2%) |
| HLT: Ischaemic coronary artery disorders | 0 | | 1 | (1.2%) | 1 | (0.6%) | 2 | (0.8%) | 2 | (0.8%) | 4 | (0.8%) |
| Acute myocardial infarction | 0 | | 1 | (1.2%) | 1 | (0.6%) | 1 | (0.4%) | 1 | (0.4%) | 2 | (0.4%) |
| Angina unstable | 0 | | 1 | (1.2%) | 1 | (0.6%) | 1 | (0.4%) | 1 | (0.4%) | 2 | (0.4%) |
| HLGT: Heart failures | 0 | | 1 | (1.2%) | 1 | (0.6%) | 0 | | 1 | (0.4%) | 1 | (0.2%) |
| HLT: Heart failures NEC | 0 | | 1 | (1.2%) | 1 | (0.6%) | 0 | | 1 | (0.4%) | 1 | (0.2%) |
| Cardiac failure congestive | 0 | | 1 | (1.2%) | 1 | (0.6%) | 0 | | 1 | (0.4%) | 1 | (0.2%) |
| HLGT: Myocardial disorders | 0 | | 0 | | 0 | | 0 | | 1 | (0.4%) | 1 | (0.2%) |
| HLT: Cardiomyopathies | 0 | | 0 | | 0 | | 0 | | 1 | (0.4%) | 1 | (0.2%) |
| Cardiomyopathy | 0 | | 0 | | 0 | | 0 | | 1 | (0.4%) | 1 | (0.2%) |
| VASCULAR DISORDERS | 0 | | 0 | | 0 | | 1 | (0.4%) | 3 | (1.2%) | 4 | (0.8%) |
| HLGT: Arteriosclerosis, stenosis, vascular | 0 | | 0 | | 0 | | 0 | | 1 | (0.4%) | 1 | (0.2%) |
| insufficiency and necrosis | | | | | | | | | | | | |
| HLT: Peripheral vasoconstriction, necrosis | 0 | | 0 | | 0 | | 0 | | 1 | (0.4%) | 1 | (0.2%) |
| and vascular insufficiency | | | | | | | | | | | | |
| Peripheral arterial occlusive disease | 0 | | 0 | | 0 | | 0 | | 1 | (0.4%) | 1 | (0.2%) |
| HLGT: Vascular hypertensive disorders | 0 | | 0 | | 0 | | 1 | (0.4%) | 2 | (0.8%) | 3 | (0.6%) |
| HLT: Accelerated and malignant | 0 | | 0 | | 0 | | 1 | (0.4%) | 2 | (0.8%) | 3 | (0.6%) |
| hypertension | | | | | | | | | | | | |
| Hypertensive crisis | 0 | | 0 | | 0 | | 1 | (0.4%) | 2 | (0.8%) | 3 | (0.6%) |
| RESPIRATORY, THORACIC AND | 0 | | 0 | | 0 | | 0 | | 2 | (0.8%) | 2 | (0.4%) |
| MEDIASTINAL DISORDERS | | | | | | | | | | | | |
| HLGT: Bronchial disorders (excl neoplasms) | 0 | | 0 | | 0 | | 0 | | 1 | (0.4%) | 1 | (0.2%) |
| HLT: Bronchospasm and obstruction | 0 | | 0 | | 0 | | 0 | | 1 | (0.4%) | 1 | (0.2%) |
| Bronchial obstruction | 0 | | 0 | | 0 | | 0 | | 1 | (0.4%) | 1 | (0.2%) |
| HLGT: Lower respiratory tract disorders (excl | 0 | | 0 | | 0 | | 0 | | 1 | (0.4%) | 1 | (0.2%) |
| obstruction and infection) | | | | | | | | | | | | |
| HLT: Pulmonary oedemas | 0 | | 0 | | 0 | | 0 | | 1 | (0.4%) | 1 | (0.2%) |
| Pulmonary oedema | 0 | | 0 | | 0 | | 0 | | 1 | (0.4%) | 1 | (0.2%) |
| GASTROINTESTINAL DISORDERS | 1 | (1.2%) | 0 | | 1 | (0.6%) | 2 | (0.8%) | 4 | (1.6%) | 6 | (1.2%) |
| HLGT: Abdominal hernias and other abdominal | 0 | | 0 | | 0 | | 1 | (0.4%) | 1 | (0.4%) | 2 | (0.4%) |
| wall conditions | | | | | | | | | | | | |
| HLT: Inguinal hernias | 0 | | 0 | | 0 | | 1 | (0.4%) | 1 | (0.4%) | 2 | (0.4%) |
| Inguinal hernia | 0 | | 0 | | 0 | | 0 | | 1 | (0.4%) | 1 | (0.2%) |
| Inguinal hernia, obstructive | 0 | | 0 | | 0 | | 1 | (0.4%) | 0 | | 1 | (0.2%) |
| HLGT: Benign neoplasms gastrointestinal | 0 | | 0 | | 0 | | 0 | | 1 | (0.4%) | 1 | (0.2%) |
| HLT: Benign neoplasms gastrointestinal | 0 | | 0 | | 0 | | 0 | | 1 | (0.4%) | 1 | (0.2%) |
| (excl oral cavity) | | | | | | | | | | | | |
| Rectal polyp | 0 | | 0 | | 0 | | 0 | | 1 | (0.4%) | 1 | (0.2%) |
| HLGT: Gastrointestinal haemorrhages NEC | 1 | (1.2%) | 0 | | 1 | (0.6%) | 0 | | 0 | | 0 | |
| HLT: Gastric and oesophageal haemorrhages | 1 | (1.2%) | 0 | | 1 | (0.6%) | 0 | | 0 | | 0 | |
| Gastric haemorrhage | 1 | (1.2%) | 0 | | 1 | (0.6%) | 0 | | 0 | | 0 | |
| HLGT: Gastrointestinal inflammatory conditions | 0 | | 0 | | 0 | | 0 | | 1 | (0.4%) | 1 | (0.2%) |
| HLT: Gastritis (excl infective) | 0 | | 0 | | 0 | | 0 | | 1 | (0.4%) | 1 | (0.2%) |
| Gastritis | 0 | | 0 | | 0 | | 0 | | 1 | (0.4%) | 1 | (0.2%) |
| HLGT: Gastrointestinal vascular conditions | 0 | | 0 | | 0 | | 1 | (0.4%) | 0 | | 1 | (0.2%) |
| HLT: Haemorrhoids and gastrointestinal | 0 | | 0 | | 0 | | 1 | (0.4%) | 0 | | 1 | (0.2%) |
| varices (excl oesophageal) | | | | | | | | | | | | |
| Haemorrhoids | 0 | | 0 | | 0 | | 1 | (0.4%) | 0 | | 1 | (0.2%) |
| HLGT: Peritoneal and retroperitoneal conditions | 0 | | 0 | | 0 | | 0 | | 1 | (0.4%) | 1 | (0.2%) |
| HLT: Peritoneal and retroperitoneal fibrosis | 0 | | 0 | | 0 | | 0 | | 1 | (0.4%) | 1 | (0.2%) |
| and adhesions | | | | | | | | | | | | |
| Abdominal adhesions | 0 | | 0 | | 0 | | 0 | | 1 | (0.4%) | 1 | (0.2%) |
| HEPATOBILIARY DISORDERS | 0 | | 2 | (2.4%) | 2 | (1.2%) | 1 | (0.4%) | 0 | | 1 | (0.2%) |
| HLGT: Gallbladder disorders | 0 | | 2 | (2.4%) | 2 | (1.2%) | 1 | (0.4%) | 0 | | 1 | (0.2%) |
| HLT: Cholecystitis and cholelithiasis | 0 | | 2 | (2.4%) | 2 | (1.2%) | 1 | (0.4%) | 0 | | 1 | (0.2%) |
| Cholecystitis | 0 | | 0 | | 0 | | 1 | (0.4%) | 0 | | 1 | (0.2%) |
| Cholecystitis acute | 0 | | 1 | (1.2%) | 1 | (0.6%) | 0 | | 0 | | 0 | |
| Cholelithiasis | 0 | | 1 | (1.2%) | 1 | (0.6%) | 0 | | 0 | | 0 | |
| SKIN AND SUBCUTANEOUS TISSUE | 0 | | 0 | | 0 | | 1 | (0.4%) | 0 | | 1 | (0.2%) |
| DISORDERS | | | | | | | | | | | | |
| HLGT: Angioedema and urticaria | 0 | | 0 | | 0 | | 1 | (0.4%) | 0 | | 1 | (0.2%) |
| HLT: Angioedemas | 0 | | 0 | | 0 | | 1 | (0.4%) | 0 | | 1 | (0.2%) |
| Angioedema | 0 | | 0 | | 0 | | 1 | (0.4%) | 0 | | 1 | (0.2%) |
| HLGT: Epidermal and dermal conditions | 0 | | 0 | | 0 | | 1 | (0.4%) | 0 | | 1 | (0.2%) |
| HLT: Rashes, eruptions and exanthems NEC | 0 | | 0 | | 0 | | 1 | (0.4%) | 0 | | 1 | (0.2%) |
| Rash maculo-papular | 0 | | 0 | | 0 | | 1 | (0.4%) | 0 | | 1 | (0.2%) |
| MUSCULOSKELETAL AND CONNECTIVE | 0 | | 0 | | 0 | | 1 | (0.4%) | 3 | (1.2%) | 4 | (0.8%) |
| TISSUE DISORDERS | | | | | | | | | | | | |
| HLGT: Joint disorders | 0 | | 0 | | 0 | | 1 | (0.4%) | 3 | (1.2%) | 4 | (0.8%) |
| HLT: Osteoarthropathies | 0 | | 0 | | 0 | | 1 | (0.4%) | 3 | (1.2%) | 4 | (0.8%) |
| Osteoarthritis | 0 | | 0 | | 0 | | 1 | (0.4%) | 3 | (1.2%) | 4 | (0.8%) |
| RENAL AND URINARY DISORDERS | 0 | | 1 | (1.2%) | 1 | (0.6%) | 0 | | 0 | | 0 | |
| HLGT: Urolithiases | 0 | | 1 | (1.2%) | 1 | (0.6%) | 0 | | 0 | | 0 | |
| HLT: Renal lithiasis | 0 | | 1 | (1.2%) | 1 | (0.6%) | 0 | | 0 | | 0 | |
| Nephrolithiasis | 0 | | 1 | (1.2%) | 1 | (0.6%) | 0 | | 0 | | 0 | |
| GENERAL DISORDERS AND | 0 | | 0 | | 0 | | 0 | | 1 | (0.4%) | 1 | (0.2%) |
| ADMINISTRATION SITE CONDITIONS | | | | | | | | | | | | |
| HLGT: Tissue disorders NEC | 0 | | 0 | | 0 | | 0 | | 1 | (0.4%) | 1 | (0.2%) |
| HLT: Necrosis NEC | 0 | | 0 | | 0 | | 0 | | 1 | (0.4%) | 1 | (0.2%) |
| Necrobiosis | 0 | | 0 | | 0 | | 0 | | 1 | (0.4%) | 1 | (0.2%) |
| INVESTIGATIONS | 0 | | 0 | | 0 | | 0 | | 1 | (0.4%) | 1 | (0.2%) |
| HLGT: Gastrointestinal investigations | 0 | | 0 | | 0 | | 0 | | 1 | (0.4%) | 1 | (0.2%) |
| HLT: Digestive enzymes | 0 | | 0 | | 0 | | 0 | | 1 | (0.4%) | 1 | (0.2%) |
| Pancreatic enzymes increased | 0 | | 0 | | 0 | | 0 | | 1 | (0.4%) | 1 | (0.2%) |
| INJURY, POISONING AND PROCEDURAL | 0 | | 1 | (1.2%) | 1 | (0.6%) | 2 | (0.8%) | 4 | (1.6%) | 6 | (1.2%) |
| COMPLICATIONS | | | | | | | | | | | | |
| HLGT: Bone and joint injuries | 0 | | 1 | (1.2%) | 1 | (0.6%) | 2 | (0.8%) | 4 | (1.6%) | 6 | (1.2%) |
| HLT: Limb injuries NEC (incl traumatic | 0 | | 1 | (1.2%) | 1 | (0.6%) | 0 | | 0 | | 0 | |
| amputation) | | | | | | | | | | | | |
| Joint injury | 0 | | 1 | (1.2%) | 1 | (0.6%) | 0 | | 0 | | 0 | |
| HLT: Skull fractures, facial bone fractures | 0 | | 0 | | 0 | | 0 | | 1 | (0.4%) | 1 | (0.2%) |
| and dislocations | | | | | | | | | | | | |
| Skull fracture | 0 | | 0 | | 0 | | 0 | | 1 | (0.4%) | 1 | (0.2%) |
| HLT: Spinal fractures and dislocations | 0 | | 0 | | 0 | | 1 | (0.4%) | 1 | (0.4%) | 2 | (0.4%) |
| Dislocation of vertebra | 0 | | 0 | | 0 | | 0 | | 1 | (0.4%) | 1 | (0.2%) |
| Spinal fracture | 0 | | 0 | | 0 | | 1 | (0.4%) | 0 | | 1 | (0.2%) |
| HLT: Thoracic cage fractures and | 0 | | 0 | | 0 | | 1 | (0.4%) | 0 | | 1 | (0.2%) |
| dislocations | | | | | | | | | | | | |
| Rib fracture | 0 | | 0 | | 0 | | 1 | (0.4%) | 0 | | 1 | (0.2%) |
| HLT: Upper limb fractures and dislocations | 0 | | 0 | | 0 | | 0 | | 2 | (0.8%) | 2 | (0.4%) |
| Humerus fracture | 0 | | 0 | | 0 | | 0 | | 1 | (0.4%) | 1 | (0.2%) |
| Wrist fracture | 0 | | 0 | | 0 | | 0 | | 1 | (0.4%) | 1 | (0.2%) |
| HLGT: Injuries NEC | 0 | | 0 | | 0 | | 1 | (0.4%) | 1 | (0.4%) | 2 | (0.4%) |
| HLT: Non-site specific injuries NEC | 0 | | 0 | | 0 | | 0 | | 1 | (0.4%) | 1 | (0.2%) |
| Fall | 0 | | 0 | | 0 | | 0 | | 1 | (0.4%) | 1 | (0.2%) |
| HLT: Spinal cord injuries NEC | 0 | | 0 | | 0 | | 1 | (0.4%) | 0 | | 1 | (0.2%) |
| Spinal cord injury | 0 | | 0 | | 0 | | 1 | (0.4%) | 0 | | 1 | (0.2%) |
| SURGICAL AND MEDICAL PROCEDURES | 0 | | 0 | | 0 | | 0 | | 1 | (0.4%) | 1 | (0.2%) |
| HLGT: Cardiac therapeutic procedures | 0 | | 0 | | 0 | | 0 | | 1 | (0.4%) | 1 | (0.2%) |
| HLT: Cardiac device therapeutic procedures | 0 | | 0 | | 0 | | 0 | | 1 | (0.4%) | 1 | (0.2%) |
| Cardiac pacemaker insertion | 0 | | 0 | | 0 | | 0 | | 1 | (0.4%) | 1 | (0.2%) |
| HLGT: Vascular therapeutic procedures | 0 | | 0 | | 0 | | 0 | | 1 | (0.4%) | 1 | (0.2%) |
| HLT: Arterial therapeutic procedures (excl | 0 | | 0 | | 0 | | 0 | | 1 | (0.4%) | 1 | (0.2%) |
| aortic) | | | | | | | | | | | | |
| Coronary artery bypass | 0 | | 0 | | 0 | | 0 | | 1 | (0.4%) | 1 | (0.2%) |

TEAE: Treatment emergent adverse event,
SOC: System Organ Class,
HLGT: High Level Group Term,
HLT: High Level Term,
PT: Preferred Term.
On-treatment period of the whole study = the time from the first dose of double-blind study medication up to 3 days after the last dose administration.
MedDRA version: 13.1.
n (%) = number and percentage of patients with at least one serious TEAE.
Note:
Table sorted by SOC internationally agreed order and HLGT, HLT, PT alphabetic order.

TABLE 22
Number (%) of patients experiencing TEAE(s) leading to permanent treatment discontinuation by primary SOC,
HLGT, HLT, and PT during the on-treatment period of the whole study - Safety population

| PRIMARY SYSTEM ORGAN CLASS | Placebo | | | Lixisenatide | | | | | |
|---|---|---|---|---|---|---|---|---|---|
| HLGT: High Level Group Term | Morning | Evening | | Morning | | Evening | | | |
| HLT: High Level Term | Injection | Injection | Combined | Injection | | Injection | | Combined | |
| Preferred Term | (N = 85) | (N = 85) | (N = 170) | (N = 255) | | (N = 255) | | (N = 510) | |
| Any class | 3 (3.5%) | 3 (3.5%) | 6 (3.5%) | 21 | (8.2%) | 24 | (9.4%) | 45 | (8.8%) |
| INFECTIONS AND INFESTATIONS | 0 | 0 | 0 | 0 | | 1 | (0.4%) | 1 | (0.2%) |
| HLGT: Bacterial infectious disorders | 0 | 0 | 0 | 0 | | 1 | (0.4%) | 1 | (0.2%) |
| HLT: Staphylococcal infections | 0 | 0 | 0 | 0 | | 1 | (0.4%) | 1 | (0.2%) |
| Staphylococcal sepsis | 0 | 0 | 0 | 0 | | 1 | (0.4%) | 1 | (0.2%) |
| NEOPLASMS BENIGN, MALIGNANT AND | 1 (1.2%) | 1 (1.2%) | 2 (1.2%) | 0 | | 0 | | 0 | |
| UNSPECIFIED (INCL CYSTS AND POLYPS) | | | | | | | | | |
| HLGT: Gastrointestinal neoplasms malignant and | 0 | 1 (1.2%) | 1 (0.6%) | 0 | | 0 | | 0 | |
| unspecified | | | | | | | | | |
| HLT: Rectal neoplasms malignant | 0 | 1 (1.2%) | 1 (0.6%) | 0 | | 0 | | 0 | |
| Rectal cancer | 0 | 1 (1.2%) | 1 (0.6%) | 0 | | 0 | | 0 | |
| HLGT: Miscellaneous and site unspecified | 1 (1.2%) | 0 | 1 (0.6%) | 0 | | 0 | | 0 | |
| neoplasms malignant and unspecified | | | | | | | | | |
| HLT: Neoplasms malignant site unspecified | 1 (1.2%) | 0 | 1 (0.6%) | 0 | | 0 | | 0 | |
| NEC | | | | | | | | | |
| Signet-ring cell carcinoma | 1 (1.2%) | 0 | 1 (0.6%) | 0 | | 0 | | 0 | |
| BLOOD AND LYMPHATIC SYSTEM | 0 | 0 | 0 | 0 | | 1 | (0.4%) | 1 | (0.2%) |
| DISORDERS | | | | | | | | | |
| HLGT: Platelet disorders | 0 | 0 | 0 | 0 | | 1 | (0.4%) | 1 | (0.2%) |
| HLT: Thrombocytopenias | 0 | 0 | 0 | 0 | | 1 | (0.4%) | 1 | (0.2%) |
| Thrombocytopenia | 0 | 0 | 0 | 0 | | 1 | (0.4%) | 1 | (0.2%) |
| METABOLISM AND NUTRITION DISORDERS | 0 | 0 | 0 | 1 | (0.4%) | 4 | (1.6%) | 5 | (1.0%) |
| HLGT: Appetite and general nutritional disorders | 0 | 0 | 0 | 0 | | 1 | (0.4%) | 1 | (0.2%) |
| HLT: General nutritional disorders NEC | 0 | 0 | 0 | 0 | | 1 | (0.4%) | 1 | (0.2%) |
| Obesity | 0 | 0 | 0 | 0 | | 1 | (0.4%) | 1 | (0.2%) |
| HLGT: Glucose metabolism disorders (incl | 0 | 0 | 0 | 1 | (0.4%) | 3 | (1.2%) | 4 | (0.8%) |
| diabetes mellitus) | | | | | | | | | |
| HLT: Hypoglycaemic conditions NEC | 0 | 0 | 0 | 1 | (0.4%) | 3 | (1.2%) | 4 | (0.8%) |
| Hypoglycaemia | 0 | 0 | 0 | 1 | (0.4%) | 3 | (1.2%) | 4 | (0.8%) |
| PSYCHIATRIC DISORDERS | 0 | 0 | 0 | 1 | (0.4%) | 0 | | 1 | (0.2%) |
| HLGT: Depressed mood disorders and | 0 | 0 | 0 | 1 | (0.4%) | 0 | | 1 | (0.2%) |
| disturbances | | | | | | | | | |
| HLT: Depressive disorders | 0 | 0 | 0 | 1 | (0.4%) | 0 | | 1 | (0.2%) |
| Depression | 0 | 0 | 0 | 1 | (0.4%) | 0 | | 1 | (0.2%) |
| NERVOUS SYSTEM DISORDERS | 0 | 0 | 0 | 3 | (1.2%) | 1 | (0.4%) | 4 | (0.8%) |
| HLGT: Headaches | 0 | 0 | 0 | 2 | (0.8%) | 1 | (0.4%) | 3 | (0.6%) |
| HLT: Headaches NEC | 0 | 0 | 0 | 1 | (0.4%) | 1 | (0.4%) | 2 | (0.4%) |
| Headache | 0 | 0 | 0 | 1 | (0.4%) | 1 | (0.4%) | 2 | (0.4%) |
| HLT: Migraine headaches | 0 | 0 | 0 | 1 | (0.4%) | 0 | | 1 | (0.2%) |
| Migraine | 0 | 0 | 0 | 1 | (0.4%) | 0 | | 1 | (0.2%) |
| HLGT: Neurological disorders NEC | 0 | 0 | 0 | 1 | (0.4%) | 0 | | 1 | (0.2%) |
| HLT: Neurological signs and symptoms NEC | 0 | 0 | 0 | 1 | (0.4%) | 0 | | 1 | (0.2%) |
| Dizziness | 0 | 0 | 0 | 1 | (0.4%) | 0 | | 1 | (0.2%) |
| EYE DISORDERS | 0 | 0 | 0 | 0 | | 1 | (0.4%) | 1 | (0.2%) |
| HLGT: Ocular infections, irritations and | 0 | 0 | 0 | 0 | | 1 | (0.4%) | 1 | (0.2%) |
| inflammations | | | | | | | | | |
| HLT: Ocular infections, inflammations and | 0 | 0 | 0 | 0 | | 1 | (0.4%) | 1 | (0.2%) |
| associated manifestations | | | | | | | | | |
| Eye irritation | 0 | 0 | 0 | 0 | | 1 | (0.4%) | 1 | (0.2%) |
| CARDIAC DISORDERS | 0 | 0 | 0 | 0 | | 1 | (0.4%) | 1 | (0.2%) |
| HLGT: Heart failures | 0 | 0 | 0 | 0 | | 1 | (0.4%) | 1 | (0.2%) |
| HLT: Heart failures NEC | 0 | 0 | 0 | 0 | | 1 | (0.4%) | 1 | (0.2%) |
| Cardiac failure congestive | 0 | 0 | 0 | 0 | | 1 | (0.4%) | 1 | (0.2%) |
| GASTROINTESTINAL DISORDERS | 1 (1.2%) | 0 | 1 (0.6%) | 11 | (4.3%) | 10 | (3.9%) | 21 | (4.1%) |
| HLGT: Exocrine pancreas conditions | 0 | 0 | 0 | 0 | | 1 | (0.4%) | 1 | (0.2%) |
| HLT: Acute and chronic pancreatitis | 0 | 0 | 0 | 0 | | 1 | (0.4%) | 1 | (0.2%) |
| Pancreatitis | 0 | 0 | 0 | 0 | | 1 | (0.4%) | 1 | (0.2%) |
| HLGT: Gastrointestinal haemorrhages NEC | 0 | 0 | 0 | 1 | (0.4%) | 0 | | 1 | (0.2%) |
| HLT: Non-site specific gastrointestinal | 0 | 0 | 0 | 1 | (0.4%) | 0 | | 1 | (0.2%) |
| haemorrhages | | | | | | | | | |
| Haematochezia | 0 | 0 | 0 | 1 | (0.4%) | 0 | | 1 | (0.2%) |
| HLGT: Gastrointestinal motility and defaecation | 0 | 0 | 0 | 1 | (0.4%) | 0 | | 1 | (0.2%) |
| conditions | | | | | | | | | |
| HLT: Diarrhoea (excl infective) | 0 | 0 | 0 | 1 | (0.4%) | 0 | | 1 | (0.2%) |
| Diarrhoea | 0 | 0 | 0 | 1 | (0.4%) | 0 | | 1 | (0.2%) |
| HLGT: Gastrointestinal signs and symptoms | 1 (1.2%) | 0 | 1 (0.6%) | 9 | (3.5%) | 9 | (3.5%) | 18 | (3.5%) |
| HLT: Flatulence, bloating and distension | 0 | 0 | 0 | 1 | (0.4%) | 0 | | 1 | (0.2%) |
| Flatulence | 0 | 0 | 0 | 1 | (0.4%) | 0 | | 1 | (0.2%) |
| HLT: Gastrointestinal and abdominal pains | 0 | 0 | 0 | 2 | (0.8%) | 0 | | 2 | (0.4%) |
| (excl oral and throat) | | | | | | | | | |
| Abdominal pain | 0 | 0 | 0 | 1 | (0.4%) | 0 | | 1 | (0.2%) |
| Abdominal pain upper | 0 | 0 | 0 | 1 | (0.4%) | 0 | | 1 | (0.2%) |
| HLT: Nausea and vomiting symptoms | 1 (1.2%) | 0 | 1 (0.6%) | 6 | (2.4%) | 9 | (3.5%) | 15 | (2.9%) |
| Nausea | 0 | 0 | 0 | 6 | (2.4%) | 7 | (2.7%) | 13 | (2.5%) |
| Vomiting | 0 | 0 | 0 | 2 | (0.8%) | 5 | (2.0%) | 7 | (1.4%) |
| Vomiting projectile | 1 (1.2%) | 0 | 1 (0.6%) | 0 | | 0 | | 0 | |
| HEPATOBILIARY DISORDERS | 0 | 1 (1.2%) | 1 (0.6%) | 0 | | 1 | (0.4%) | 1 | (0.2%) |
| HLGT: Bile duct disorders | 0 | 0 | 0 | 0 | | 1 | (0.4%) | 1 | (0.2%) |
| HLT: Bile duct infections and inflammations | 0 | 0 | 0 | 0 | | 1 | (0.4%) | 1 | (0.2%) |
| Biliary colic | 0 | 0 | 0 | 0 | | 1 | (0.4%) | 1 | (0.2%) |
| HLGT: Gallbladder disorders | 0 | 1 (1.2%) | 1 (0.6%) | 0 | | 0 | | 0 | |
| HLT: Cholecystitis and cholelithiasis | 0 | 1 (1.2%) | 1 (0.6%) | 0 | | 0 | | 0 | |
| Cholecystitis acute | 0 | 1 (1.2%) | 1 (0.6%) | 0 | | 0 | | 0 | |
| SKIN AND SUBCUTANEOUS TISSUE | 0 | 0 | 0 | 2 | (0.8%) | 2 | (0.8%) | 4 | (0.8%) |
| DISORDERS | | | | | | | | | |
| HLGT: Angioedema and urticaria | 0 | 0 | 0 | 1 | (0.4%) | 0 | | 1 | (0.2%) |
| HLT: Angioedemas | 0 | 0 | 0 | 1 | (0.4%) | 0 | | 1 | (0.2%) |
| Angioedema | 0 | 0 | 0 | 1 | (0.4%) | 0 | | 1 | (0.2%) |
| HLGT: Epidermal and dermal conditions | 0 | 0 | 0 | 1 | (0.4%) | 2 | (0.8%) | 3 | (0.6%) |
| HLT: Dermatitis and eczema | 0 | 0 | 0 | 0 | | 2 | (0.8%) | 2 | (0.4%) |
| Dermatitis allergic | 0 | 0 | 0 | 0 | | 2 | (0.8%) | 2 | (0.4%) |
| HLT: Rashes, eruptions and exanthems NEC | 0 | 0 | 0 | 1 | (0.4%) | 0 | | 1 | (0.2%) |
| Rash maculo-papular | 0 | 0 | 0 | 1 | (0.4%) | 0 | | 1 | (0.2%) |
| HLGT: Skin appendage conditions | 0 | 0 | 0 | 1 | (0.4%) | 0 | | 1 | (0.2%) |
| HLT: Rosaceas | 0 | 0 | 0 | 1 | (0.4%) | 0 | | 1 | (0.2%) |
| Rosacea | 0 | 0 | 0 | 1 | (0.4%) | 0 | | 1 | (0.2%) |
| MUSCULOSKELETAL AND CONNECTIVE | 0 | 0 | 0 | 0 | | 1 | (0.4%) | 1 | (0.2%) |
| TISSUE DISORDERS | | | | | | | | | |
| HLGT: Musculoskeletal and connective tissue | 0 | 0 | 0 | 0 | | 1 | (0.4%) | 1 | (0.2%) |
| disorders NEC | | | | | | | | | |
| HLT: Musculoskeletal and connective tissue | 0 | 0 | 0 | 0 | | 1 | (0.4%) | 1 | (0.2%) |
| pain and discomfort | | | | | | | | | |
| Back pain | 0 | 0 | 0 | 0 | | 1 | (0.4%) | 1 | (0.2%) |
| RENAL AND URINARY DISORDERS | 0 | 1 (1.2%) | 1 (0.6%) | 0 | | 0 | | 0 | |
| HLGT: Renal disorders (excl nephropathies) | 0 | 1 (1.2%) | 1 (0.6%) | 0 | | 0 | | 0 | |
| HLT: Renal failure and impairment | 0 | 1 (1.2%) | 1 (0.6%) | 0 | | 0 | | 0 | |
| Acute prerenal failure | 0 | 1 (1.2%) | 1 (0.6%) | 0 | | 0 | | 0 | |
| GENERAL DISORDERS AND | 0 | 0 | 0 | 1 | (0.4%) | 0 | | 1 | (0.2%) |
| ADMINISTRATION SITE CONDITIONS | | | | | | | | | |
| HLGT: General system disorders NEC | 0 | 0 | 0 | 1 | (0.4%) | 0 | | 1 | (0.2%) |
| HLT: Asthenic conditions | 0 | 0 | 0 | 1 | (0.4%) | 0 | | 1 | (0.2%) |
| Fatigue | 0 | 0 | 0 | 1 | (0.4%) | 0 | | 1 | (0.2%) |
| INVESTIGATIONS | 1 (1.2%) | 0 | 1 (0.6%) | 2 | (0.8%) | 5 | (2.0%) | 7 | (1.4%) |
| HLGT: Cardiac and vascular investigations (excl | 1 (1.2%) | 0 | 1 (0.6%) | 0 | | 0 | | 0 | |
| enzyme tests) | | | | | | | | | |
| HLT: ECG investigations | 1 (1.2%) | 0 | 1 (0.6%) | 0 | | 0 | | 0 | |
| Electrocardiogram abnormal | 1 (1.2%) | 0 | 1 (0.6%) | 0 | | 0 | | 0 | |
| HLGT: Endocrine investigations (incl sex | 0 | 0 | 0 | 1 | (0.4%) | 2 | (0.8%) | 3 | (0.6%) |
| hormones) | | | | | | | | | |
| HLT: Gastrointestinal, pancreatic and APUD | 0 | 0 | 0 | 1 | (0.4%) | 2 | (0.8%) | 3 | (0.6%) |
| hormone analyses | | | | | | | | | |
| Blood calcitonin increased | 0 | 0 | 0 | 1 | (0.4%) | 2 | (0.8%) | 3 | (0.6%) |
| HLGT: Gastrointestinal investigations | 0 | 0 | 0 | 1 | (0.4%) | 2 | (0.8%) | 3 | (0.6%) |
| HLT: Digestive enzymes | 0 | 0 | 0 | 1 | (0.4%) | 2 | (0.8%) | 3 | (0.6%) |
| Blood amylase increased | 0 | 0 | 0 | 0 | | 1 | (0.4%) | 1 | (0.2%) |
| Lipase increased | 0 | 0 | 0 | 1 | (0.4%) | 1 | (0.4%) | 2 | (0.4%) |
| Pancreatic enzymes increased | 0 | 0 | 0 | 0 | | 1 | (0.4%) | 1 | (0.2%) |
| HLGT: Hepatobiliary investigations | 0 | 0 | 0 | 0 | | 1 | (0.4%) | 1 | (0.2%) |
| HLT: Liver function analyses | 0 | 0 | 0 | 0 | | 1 | (0.4%) | 1 | (0.2%) |
| Hepatic enzyme increased | 0 | 0 | 0 | 0 | | 1 | (0.4%) | 1 | (0.2%) |
| INJURY, POISONING AND PROCEDURAL | 0 | 0 | 0 | 2 | (0.8%) | 0 | | 2 | (0.4%) |
| COMPLICATIONS | | | | | | | | | |
| HLGT: Bone and joint injuries | 0 | 0 | 0 | 1 | (0.4%) | 0 | | 1 | (0.2%) |
| HLT: Spinal fractures and dislocations | 0 | 0 | 0 | 1 | (0.4%) | 0 | | 1 | (0.2%) |
| Spinal fracture | 0 | 0 | 0 | 1 | (0.4%) | 0 | | 1 | (0.2%) |
| HLGT: Injuries NEC | 0 | 0 | 0 | 2 | (0.8%) | 0 | | 2 | (0.4%) |
| HLT: Cerebral injuries NEC | 0 | 0 | 0 | 1 | (0.4%) | 0 | | 1 | (0.2%) |
| Concussion | 0 | 0 | 0 | 1 | (0.4%) | 0 | | 1 | (0.2%) |
| HLT: Muscle, tendon and ligament injuries | 0 | 0 | 0 | 1 | (0.4%) | 0 | | 1 | (0.2%) |
| Muscle strain | 0 | 0 | 0 | 1 | (0.4%) | 0 | | 1 | (0.2%) |
| HLT: Site specific injuries NEC | 0 | 0 | 0 | 1 | (0.4%) | 0 | | 1 | (0.2%) |
| Head injury | 0 | 0 | 0 | 1 | (0.4%) | 0 | | 1 | (0.2%) |
| Mouth injury | 0 | 0 | 0 | 1 | (0.4%) | 0 | | 1 | (0.2%) |
| HLT: Spinal cord injuries NEC | 0 | 0 | 0 | 1 | (0.4%) | 0 | | 1 | (0.2%) |
| Spinal cord injury | 0 | 0 | 0 | 1 | (0.4%) | 0 | | 1 | (0.2%) |

TEAE: Treatment emergent adverse event,
SOC: System Organ Class,
HLGT: High Level Group Term,
HLT: High Level Term,
PT: Preferred Term.
On-treatment period of the whole study = the time from the first dose of double-blind study medication up to 3 days after the last dose administration.
MedDRA version: 13.1.
n (%) = number and percentage of patients with at least one TEAE leading to permanent treatment discontinuation.
Note:
Table sorted by SOC internationally agreed order and HLGT, HLT, PT alphabetic order.

During the on-treatment period of the whole study, 18 (7.1%) patients in the lixisenatide morning injection arm, 22 (8.6%) in the lixisenatide evening injection arm reported at least one symptomatic hypoglycemia event per protocol definition, compared to 4 (2.4%) in the combined placebo group (Table 23). None of the symptomatic hypoglycemia events were severe in intensity. Twelve additional patients (5 in the lixisenatide morning injection arm, 6 in the lixisenatide morning injection arm and 1 in the combined placebo group) reported hypoglycemia (Table 32), but these events did not meet the protocol-specified definition (for all cases but 2 the associated glucose values≧60 mg/dL, 1 with no glucose value recovered without counter measurements, and 1 with symptoms recorded only).

Seventeen (6.7%) patients in each lixisenatide arm and 6 (3.5%) in the combined placebo group experienced injection site reaction AEs (Table 24). The injection site reaction AEs were identified by searching the term “injection site” in either the PTs coded from the investigator reported terms or the PTs from the ARAC diagnosis after the allergic reaction adjudication. None of these reactions was serious or severe in intensity. Only 1 event (reported as “allergic exanthema” and coded to PT “dermatitis allergic” from the investigator reported term) in the lixisenatide evening injection arm led to IP discontinuation. The event was sent to ARAC but was not adjudicated as an allergic reaction; the coded term from ARAC diagnosis was local reaction at injection site.

A total of 41 events were reported for 36 patients as possible allergic events by investigators and sent to ARAC for adjudication during the on-treatment period of the whole study. Of these, 12 events in 10 patients (3 [1.2%] patients in the lixisenatide morning injection arm, 4 [1.6%] in the lixisenatide evening injection arm, and 3 [1.8%] in the combined placebo group) were adjudicated as allergic reactions by ARAC including 3 events in 2 patients (1 with anaphylactic reaction and angioedema in the lixisenatide morning injection arm and 1 with urticaria in the lixisenatide evening injection arm) adjudicated as possibly related to IP (Table 25).
Patient #124411018 (lixisenatide morning injection), a 53 year old male, with a history of drug hypersensitivity, dyslipidaemia, hypertension, obesity, gastroesophageal reflux disease, benign prostatic hyperplasia, back pain and rhinoplasty, developed skin reactions on study Day 13. The events were reported as “maculopapular rash” and “angioedema” and coded to PT “rash maculo-papular” and “angioedema” respectively. Both events became serious the next day and IP was permanently discontinued. Corrective treatment with antihistamines and steroids was applied and events resolved 8 and 5 days respectively after onset. The events were adjudicated by ARAC respectively as an anaphylactic reaction and angioedema, both possibly related to the IP.
Patient #484401005 (lixisenatide evening injection), a 63 year old female, with a history of hysterectomy, appendicectomy, osteoporosis, hypertriglyceridemia, and caesarean section, developed a generalized itch with a rash (hives) on 27 Jun. 2009, probably related to the IP, about 6 months after starting the IP, of moderate intensity. The patient responded well to the antihistamines (chloropyramine IM and oral, and chlorphenamine oral) administered in the office. IP was discontinued on 26 Jun. 2009. This non-serious allergic event recovered within about 6 weeks. The events were adjudicated by ARAC as urticaria, possibly related to the IP.

Per protocol, any increase in amylase and/or lipase above twice the upper limit of normal range (ULN) that had been confirmed by a repeat measurement was to be monitored and documented on a pre-specified AE form for “suspected pancreatitis”. During the on-treatment period of the whole study, 3 (1.2%) patients in the lixisenatide morning injection arm, 9 (3.5%) in the lixisenatide evening injection arm, and 1 (0.6%) in the combined placebo group reported 15 TEAEs with the pre-specified AE form (Table 26). Among them, one patient in the lixisenatide evening injection arm reported suspected pancreatitis.
Patient #152402015 (lixisenatide evening injection), a 55 year old male, with a history of dyslipidaemia and hypertension, developed “suspected pancreatitis” with elevated amylase (149 U/L) and lipase (411 U/L) on Day 170 (29 Dec. 2009) after first dose of IP. The only amylase increase above 2×ULN (2.2 ULN on 29 Dec. 2009) was not confirmed at retest; afterwards, amylase fluctuated between 1.2 and 1.5×ULN. Lipase was elevated on 2 occasions: 6.5×ULN (29 Dec. 2009) and 4.2×ULN (25 Feb. 2010), but not confirmed at retest at either date; all other lipase values remained between 0.4 and 1.2×ULN. Some epigastralgia was experienced by the patient before the suspicion of pancreatitis was reported but it recovered without corrective treatment and with continuation of IP. The event was coded to PT “pancreatitis” and assessed by the investigator as related to IP which led to IP permanent discontinuation. Per available information, it seems that no additional exploration was performed to confirm the diagnosis of pancreatitis. This patient also had an idiopathic polyglobulia. Without corrective treatment, the event resolved about 2 and half months later.

Patients who had at least one value of lipase or amylase≧3 ULN during the on-treatment period are summarized in (Table 27). A total of 15 patients experienced elevated lipase (≧3 ULN): 2 [0.8%] in the lixisenatide morning injection atm, 9 [3.5%] in the lixisenatide evening injection arm, and 4 [2.4%] in the combined placebo group. Four (1.6%) patients in the lixisenatide evening injection arm and 1 patient (0.6%) in the combined placebo group had elevated amylase≧3 ULN whereas none in the lixisenatide morning injection arm did. No one had both lipase and amylase≧3 ULN during the whole study.

Per protocol, any calcitonin value≧20 pg/mL confirmed by a repeat measurement was to be monitored and reported on the pre-specified AE form for “increased calcitonin≧20 pg/mL”. During the on-treatment period of the whole study, 5 (2%) patients in the lixisenatide morning injection arm, 4 (1.6%) in the lixisenatide evening injection arm and 3 (1.8%) in the combined placebo group reported TEAEs with the pre-specified AE form (Table 28). Among the 8 lixisenatide-treated who experienced increased blood calcitonin, 1 patient in the evening arm had calcitonin value≧50 ng/L, 3 in each morning and evening injection arm had calcitonin values≧20 but <50 ng/L, and 1 in the morning arm had calcitonin value≦20 ng/L. In the combined placebo group, 1 patient had had calcitonin<20 ng/L and 2 patients had calcitonin value≧20 but<50 ng/L. In addition, 1 patient in the lixisenatide evening injection arm was diagnosed with node in left lobe of thyroid gland coded to PT “thyroid neoplasm”. Her calcitonin level was reported <20 ng/L and the event was assessed as not related to the IP by the investigator.
Patient #203402006 (lixisenatide evening injection), a 64 year old female with a history of frequent bronchitis, obesity, hypertension, dyslipidemia, atrial fibrillation, and hypercoagulation had elevated calcitonin values (108 ng/L, 21.7 ULN) on V3 (at randomization) after 1^{st} dose of IP, which leads to IP discontinuation around 4 months after. Her calcitonin level remained above 100 ng/L after IP discontinuation. She was regularly followed up by a thyroid specialist and underwent several specific explorations (ultrasound: chronic lymphocytic thyroiditis without goiter with hypothyreosis).

A total of 4 patients (0.6%) in the lixisenatide combined group reported a TEAEs of thyroid neoplasm during the study versus none in the placebo combined group.
Patient #804403024 (lixisenatide morning injection), a 54-year-old female, with a history of hypertension and appendicectomy, developed hepatic steatosis on 10 Sep. 2010 about 13 months after randomization, and was hospitalized from 19 Oct. to 8 Nov. 2010 for an acute myocardial infarction, A node (24×18 mm) in the left lobe of the thyroid gland was discovered on 16 Sep. 2010, by thyroid ultrasound scan; It was not related to IP, which was pursued. Calcitonin was slightly elevated throughout the study, but did not exceed 2×ULN (9.9 ng/L). This adverse event was non-serious, was coded to PT “Thyroid neoplasm”, the intensity was moderate, no corrective treatment was administered and the AE did not recover.
Patient #036412010 (lixisenatide morning injection), a 65-year-old male, with a history of hypertension, sleep apnea syndrome, asthma, prostate cancer, urinary tract infection, bilateral knee arthroplasty, and non malignant mole excision started IP on 27 Jul. 2009 and developed a right mild thyroid neoplasm on 15 Apr. 2010, which was investigated further due to persistent mildly elevated calcitonin levels: an ultrasound scan coupled with fine needle aspiration (FNA) showed multiple heterogeneous thyroid nodules in both lobes, with only benign changes and no malignancy. Approximately 7 months later (26 Nov. 2010), a repeat FNA revealed suspicion of neoplasm of either a Hurtle or medullary cell type with cytological features favoring the former. On 23 Dec. 2010, the case worsened and was assessed as “medically important”, and IP was discontinued on 29 Dec. 2010. For regulatory purpose, treatment code was broken by Pharmacovigilance: the patient received lixisenatide. Per investigator, the patient had no history of multinodular of thyroid prior study entry, and there was no family history of thyroid disease. Calcitonin had not been checked at study entry. His calcitonin value was 1.3 ULN (V3, before IP administration) and there was a minimal and non consistent upward trending of the calcitonin level (range: 1.5-1.7 ULN throughout the study). On 11 Jan. 2011, a surgical team examined the patient for a right thyroidectomy. On 15 Feb. 2011, the patient was hospitalized with a large goiter, and a right hemi-thyroidectomy was conducted: the histology reported an oncocytic (Hurtle cell) adenoma and the cellular colloid nodule was benign. Patient recovered from right thyroid nodule neoplasm on 15 Feb. 2011. Based on the temporal relationship of drug use and the diagnosis of the thyroid neoplasm, a causal relationship cannot be excluded.
Patient #124413013 (lixisenatide morning injection), a 62-year-old male, with a history of appendicectomy, erectile dysfunction, epicondylitis, and hypertension, was diagnosed a year after randomization in the study a thyroid nodule, coded with PT “thyroid neoplasm”. This was a non-serious AE of mild intensity, related to IP per investigator. However, the study treatment was pursued per protocol. Calcitonin values were normal throughout the study. There was no sign of hypo- or hyperthyroidism, and the patient was followed by the PI, as thyroid specialist. There was no record of personal or familial thyroid diseases in the medical history. The thyroid nodule recovered without corrective treatment, within less than 2 months.
Patient #152404019 (lixisenatide morning injection), a 58-year-old female, with a history of inguinal hernia, dyspepsia, varicose veins, uterine leiomyoma, hysterectomy, cholecystectomy, osteoporosis, caesarean sections, hypertension and dyslipidemia, was diagnosed with solid thyroid nodules 17 months after randomization in the study. This AE was coded with PT “thyroid neoplasm”. This was a non-serious AE of mild intensity, not related to IP per investigator. The study treatment was pursued per protocol. Calcitonin values were normal throughout the study. There was no sign of hypo- or hyperthyroidism, and no specific explorations of the thyroid had been performed yet. There was no record of personal or familial thyroid diseases in the medical history. The thyroid nodules are recovering without corrective treatment. A sporadic hyponatremia (113 mmol/L) was observed on one occasion (V25, 3 Dec. 2010), not confirmed at retest, and natremia returned to normal within a week, without explanation.

There was no AE reported on the pre-specified adverse event form for “increased calcitonin≧20 pg/mL” after the discontinuation of IP in the study.

Patients with at least one serum calcitonin measurement during the on-treatment period of the whole study are summarized in Table 29 according to the 4 pre-defined categories of calcitonin level at baseline. A total of 17 patients had calcitonin values≧20 ng/L: 4 (1.7%) patients in the lixisenatide morning injection arm, 8 (3.5%) patient in the lixisenatide evening arm and 5 (3.1%) patients in the combined placebo group. Amongst them, 10 patients (3 for lixisenatide morning injection, 4 for lixisenatide evening injection and 3 for combined placebo) reported a TEAE with the pre-specified AE form (Table 28). Five out of the 12 lixisenatide-treated patients and 3 out of the 5 placebo-treated patients had a calcitonin value≧20 ng/L but did not report a TEAE with the pre-specified AE form because of an unconfirmed elevation. In the lixisenatide-treated patients, 1 in each morning and evening injection arm had a single value≧50 ng/L and 3 in the lixisenatide evening injection arm had a single value≧20 but <50 ng/L. In the combined placebo group, 2 patients had single value≧20 but <50 ng/L and the third patient had 2 measurements≧20 but <50 ng/L but both repeated tests were <20 ng/L. It should be pointed out that calcitonin measurements were implemented in a protocol amendment after most patients were already randomized in this study. Therefore, baseline calcitonin values are not available for most patients.

TABLE 23
Summary of symptomatic hypoglycemia during the on-treatment period of the whole study - Safety population

| | Placebo | | | Lixisenatide | | |
|---|---|---|---|---|---|---|
| | Morning | Evening | | Morning | Evening | |
| | Injection | Injection | Combined | Injection | Injection | Combined |
| Type | (N = 85) | (N = 85) | (N = 170) | (N = 255) | (N = 255) | (N = 510) |
| Total patient years | 127.39 | 127.90 | 255.29 | 380.52 | 361.49 | 742.01 |
| Any symptomatic hypoglycemia | | | | | | |
| Number of patients with events, n (%) | 0 | 4 (4.7%) | 4 (2.4%) | 18 (7.1%) | 22 (8.6%) | 40 (7.8%) |
| Number of patients with events per 100 patient years^{a} | 0 | 3.1 | 1.6 | 4.7 | 6.1 | 5.4 |
| Blood glucose <60 mg/dL | | | | | | |
| Number of patients with events, n (%) | 0 | 4 (4.7%) | 4 (2.4%) | 17 (6.7%) | 22 (8.6%) | 39 (7.6%) |
| Number of patients with events per 100 patient years^{a} | 0 | 3.1 | 1.6 | 4.5 | 6.1 | 5.3 |
| No blood glucose reported | | | | | | |
| Number of patients with events, n (%) | 0 | 0 | 0 | 3 (1.2%) | 1 (0.4%) | 4 (0.8%) |
| Number of patients with events per 100 patient years^{a} | 0 | 0 | 0 | 0.8 | 0.3 | 0.5 |

Symptomatic hypoglycemia = symptomatic hypoglycemia as defined per protocol.
On-treatment period of the whole study = the time from the first dose of double-blind study medication up to 3 days after the last dose administration.
^{a}Calculated as (number of patients with events * 100 divided by total exposure + 3 days in patient years).

TABLE 24
Number (%) of patients experiencing injection site reactions during the on-treatment period of the whole
study - Safety population

| | Placebo | | | | | | Lixisenatide | | | | | |
|---|---|---|---|---|---|---|---|---|---|---|---|---|
| | Morning | | Evening | | | | Morning | | Evening | | | |
| | Injection | | Injection | | Combined | | Injection | | Injection | | Combined | |
| Preferred Term | (N = 85) | | (N = 85) | | (N = 170) | | (N = 255) | | (N = 255) | | (N = 510) | |
| Any injection site reactions | 4 | (4.7%) | 2 | (2.4%) | 6 | (3.5%) | 17 | (6.7%) | 17 | (6.7%) | 34 | (6.7%) |
| Investigator reported PTs | 3 | (3.5%) | 2 | (2.4%) | 5 | (2.9%) | 17 | (6.7%) | 15 | (5.9%) | 32 | (6.3%) |
| Injection site pain | 2 | (2.4%) | 1 | (1.2%) | 3 | (1.8%) | 6 | (2.4%) | 4 | (1.6%) | 10 | (2.0%) |
| Injection site haematoma | 1 | (1.2%) | 0 | | 1 | (0.6%) | 4 | (1.6%) | 3 | (1.2%) | 7 | (1.4%) |
| Injection site discomfort | 0 | | 0 | | 0 | | 0 | | 1 | (0.4%) | 1 | (0.2%) |
| Injection site erythema | 0 | | 0 | | 0 | | 1 | (0.4%) | 0 | | 1 | (0.2%) |
| Injection site haemorrhage | 0 | | 0 | | 0 | | 0 | | 1 | (0.4%) | 1 | (0.2%) |
| Injection site infection | 0 | | 1 | (1.2%) | 1 | (0.6%) | 0 | | 0 | | 0 | |
| Injection site irritation | 0 | | 0 | | 0 | | 1 | (0.4%) | 2 | (0.8%) | 3 | (0.6%) |
| Injection site pruritus | 0 | | 0 | | 0 | | 1 | (0.4%) | 1 | (0.4%) | 2 | (0.4%) |
| Injection site rash | 0 | | 0 | | 0 | | 2 | (0.8%) | 1 | (0.4%) | 3 | (0.6%) |
| Injection site reaction | 0 | | 0 | | 0 | | 6 | (2.4%) | 2 | (0.8%) | 8 | (1.6%) |
| PTs by ARAC diagnosis | 1 | (1.2%) | 0 | | 1 | (0.6%) | 2 | (0.8%) | 3 | (1.2%) | 5 | (1.0%) |
| Injection site reaction | 1 | (1.2%) | 0 | | 1 | (0.6%) | 1 | (0.4%) | 3 | (1.2%) | 4 | (0.8%) |
| Injection site rash | 0 | | 0 | | 0 | | 1 | (0.4%) | 0 | | 1 | (0.2%) |

PT = Preferred term.
On-treatment period of the whole study = the time from the first dose of double-blind study medication up to 3 days after the last dose administration.
ARAC = Allergic Reaction Assessment Committee.

TABLE 25
Number (%) of patients with allergic reaction adjudicated as allergic reaction by ARAC during the on-treatment period
of the whole study - Safety population

| Relationship | | | Placebo | | | Lixisenatide | | | |
|---|---|---|---|---|---|---|---|---|---|
| to study | MedDRA coded term | | Morning | Evening | | Morning | Evening | | |
| treatment | (PT) | ARAC | Injection | Injection | Combined | Injection | Injection | Combined | |
| (by ARAC) | for ARAC diagnosis | diagnosis | (N = 85) | (N = 85) | (N = 170) | (N = 255) | (N = 255) | (N = 510) | |
| All | Allergic reaction | | 2 (2.4%) | 1 (1.2%) | 3 (1.8%) | 3 (1.2%) | 4 (1.6%) | 7 | (1.4%) |
| | adjudicated as allergic | | | | | | | | |
| | reaction by ARAC | | | | | | | | |
| | Anaphylactic reaction | Anaphylactic reaction | 0 | 0 | 0 | 1 (0.4%) | 0 | 1 | (0.2%) |
| | Angioedema | Angioedema | 1 (1.2%) | 0 | 1 (0.6%) | 1 (0.4%) | 0 | 1 | (0.2%) |
| | Conjunctivitis allergic | Allergic conjunctivitis | 0 | 0 | 0 | 0 | 1 (0.4%) | 1 | (0.2%) |
| | Dermatitis atopic | Atopic dermatitis | 0 | 1 (1.2%) | 1 (0.6%) | 0 | 0 | 0 | |
| | Dermatitis contact | Contact dermatitis due | 0 | 0 | 0 | 0 | 1 (0.4%) | 1 | (0.2%) |
| | | to nickel | | | | | | | |
| | Food allergy | Rash due to sea food | 0 | 0 | 0 | 1 (0.4%) | 0 | 1 | (0.2%) |
| | Rhinitis allergic | Allergic rhinitis | 0 | 0 | 0 | 1 (0.4%) | 0 | 1 | (0.2%) |
| | Urticaria | Urticaria (hives) | 1 (1.2%) | 0 | 1 (0.6%) | 0 | 2 (0.8%) | 2 | (0.4%) |
| Possibly | Allergic reaction | | 0 | 0 | 0 | 1 (0.4%) | 1 (0.4%) | 2 | (0.4%) |
| related to IP | adjudicated as allergic | | | | | | | | |
| | reaction by ARAC | | | | | | | | |
| | Anaphylactic reaction | Anaphylactic reaction | 0 | 0 | 0 | 1 (0.4%) | 0 | 1 | (0.2%) |
| | Angioedema | Angioedema | 0 | 0 | 0 | 1 (0.4%) | 0 | 1 | (0.2%) |
| | Urticaria | Urticaria (hives) | 0 | 0 | 0 | 0 | 1 (0.4%) | 1 | (0.2%) |
| Not related | Allergic reaction | | 2 (2.4%) | 1 (1.2%) | 3 (1.8%) | 2 (0.8%) | 3 (1.2%) | 5 | (1.0%) |
| to IP | adjudicated as allergic | | | | | | | | |
| | reaction by ARAC | | | | | | | | |
| | Angioedema | Angioedema | 1 (1.2%) | 0 | 1 (0.6%) | 0 | 0 | 0 | |
| | Conjunctivitis allergic | Allergic conjunctivitis | 0 | 0 | 0 | 0 | 1 (0.4%) | 1 | (0.2%) |
| | Dermatitis atopic | Atopic dermatitis | 0 | 1 (1.2%) | 1 (0.6%) | 0 | 0 | 0 | |
| | Dermatitis contact | Contact dermatitis due | 0 | 0 | 0 | 0 | 1 (0.4%) | 1 | (0.2%) |
| | | to nickel | | | | | | | |
| | Food allergy | Rash due to sea food | 0 | 0 | 0 | 1 (0.4%) | 0 | 1 | (0.2%) |
| | Rhinitis allergic | Allergic rhinitis | 0 | 0 | 0 | 1 (0.4%) | 0 | 1 | (0.2%) |
| | Urticaria | Urticaria (hives) | 1 (1.2%) | 0 | 1 (0.6%) | 0 | 1 (0.4%) | 1 | (0.2%) |

Only rows with count of at least 1 in at least one column are shown
ARAC = Allergic Reaction Assessment Committee. IP = Investigational product.
On-treatment period of the whole study = the time from the first dose of double-blind study medication up to 3 days after the last dose administration.

TABLE 26
Number (%) of patients with a specific adverse event form for suspected pancreatitis completed
during the on-treatment period of the whole study - Safety population

| | Placebo | | | Lixisenatide | | |
|---|---|---|---|---|---|---|
| | Morning | Evening | | Morning | Evening | |
| | Injection | Injection | Combined | Injection | Injection | Combined |
| Preferred Term | (N = 85) | (N = 85) | (N = 170) | (N = 255) | (N = 255) | (N = 510) |
| Any | 0 | 1 (1.2%) | 1 (0.6%) | 3 (1.2%) | 9 (3.5%) | 12 (2.4%) |
| Blood amylase increased | 0 | 1 (1.2%) | 1 (0.6%) | 0 | 4 (1.6%) | 4 (0.8%) |
| Lipase increased | 0 | 0 | 0 | 3 (1.2%) | 4 (1.6%) | 7 (1.4%) |
| Pancreatic enzymes increased | 0 | 0 | 0 | 0 | 2 (0.8%) | 2 (0.4%) |
| Pancreatitis | 0 | 0 | 0 | 0 | 1 (0.4%) | 1 (0.2%) |

On-treatment period of the whole study = the time from the first dose of double-blind study medication up to 3 days after the last dose administration.
n (%) = number and percentage of patients with any cases reported on the AE form for suspected pancreatitis along with complementary form.

TABLE 27
Pancreatic enzymes: Number (%) of patients with abnormalities (PCSA) during the on-treatment period
of the whole study according to baseline PCSA status - Safety population

| | Placebo | | | Lixisenatide | | |
|---|---|---|---|---|---|---|
| Laboratory criteria | Morning | Evening | | Morning | Evening | |
| Baseline | Injection | Injection | Combined | Injection | Injection | Combined |
| By PCSA criteria n/N1 (%) | (N = 85) | (N = 85) | (N = 170) | (N = 255) | (N = 255) | (N = 510) |
| Lipase (IU/L) | | | | | | |
| Total* | | | | | | |
| ≧3 ULN | 1/83 (1.2%) | 3/85 (3.5%) | 4/168 (2.4%) | 2/251 (0.8%) | 9/255 (3.5%) | 11/506 (2.2%) |
| Normal/Missing | | | | | | |
| ≧3 ULN | 1/83 (1.2%) | 3/85 (3.5%) | 4/168 (2.4%) | 2/250 (0.8%) | 9/255 (3.5%) | 11/505 (2.2%) |
| Amylase (IU/L) | | | | | | |
| Total* | | | | | | |
| ≧3 ULN | 0/83 | 1/85 (1.2%) | 1/168 (0.6%) | 0/251 | 4/255 (1.6%) | 4/506 (0.8%) |
| Normal/Missing | | | | | | |
| ≧3 ULN | 0/83 | 1/85 (1.2%) | 1/168 (0.6%) | 0/251 | 4/255 (1.6%) | 4/506 (0.8%) |

Note:
PCSA: Potentially Clinically Significant Abnormalities. ULN = Upper limit of normal.
On-treatment period of the whole study = the time from the first dose of double-blind study medication up to 3 days after the last dose administration.
*Regardless of baseline.
Note:
The number (n) represents the subset of the total number of patients who met the criterion in question at least once. The denominator (/N1) for each parameter within a treatment group is the number of patients for the treatment group who had that parameter assessed post-baseline by baseline PCSA status. Only the worsening of the worst case for each patient is presented by baseline status.

TABLE 28
Number (%) of patients with increased calcitonin during the on-treatment period of the whole
study - Safety population

| | Placebo | | | Lixisenatide | | |
|---|---|---|---|---|---|---|
| | Morning | Evening | | Morning | Evening | |
| | Injection | Injection | Combined | Injection | Injection | Combined |
| Preferred Term n (%) | (N = 85) | (N = 85) | (N = 170) | (N = 255) | (N = 255) | (N = 510) |
| Any | 0 | 3 (3.5%) | 3 (1.8%) | 5 (2.0%) | 4 (1.6%) | 9 (1.8%) |
| Blood calcitonin increased | 0 | 3 (3.5%) | 3 (1.8%) | 4 (1.6%) | 4 (1.6%) | 8 (1.6%) |
| Thyroid neoplasm | 0 | 0 | 0 | 1 (0.4%) | 0 | 1 (0.2%) |

On-treatment period of the whole study = the time from the first dose of double-blind study medication up to 3 days after the last dose administration.
n (%) = number and percentage of patients with any cases reported on the AE form for increased calcitonin ≧20 pg/ml.

TABLE 29
Calcitonin: Number (%) of patients by pre-defined category during the on-treatment period of the whole study according to
baseline category - Safety population

| | Placebo | | | | | | Lixisenatide | | | | | |
|---|---|---|---|---|---|---|---|---|---|---|---|---|
| Laboratory criteria | Morning | | Evening | | | | Morning | | Evening | | | |
| Baseline status | Injection | | Injection | | Combined | | Injection | | Injection | | Combined | |
| Post-baseline | (N = 85) | | (N = 85) | | (N = 170) | | (N = 255) | | (N = 255) | | (N = 510) | |
| Calcitonin (ng/L) | | | | | | | | | | | | |
| Total* | | | | | | | | | | | | |
| ≦ULN | 71/79 | (89.9%) | 70/81 | (86.4%) | 141/160 | (88.1%) | 198/232 | (85.3%) | 191/227 | (84.1%) | 389/459 | (84.7%) |
| >ULN-<20 ng/L | 6/79 | (7.6%) | 8/81 | (9.9%) | 14/160 | (8.8%) | 30/232 | (12.9%) | 28/227 | (12.3%) | 58/459 | (12.6%) |
| ≧20 ng/L-<50 ng/L | 2/79 | (2.5%) | 3/81 | (3.7%) | 5/160 | (3.1%) | 3/232 | (1.3%) | 6/227 | (2.6%) | 9/459 | (2.0%) |
| ≧50 ng/L | 0/79 | | 0/81 | | 0/160 | | 1/232 | (0.4%) | 2/227 | (0.9%) | 3/459 | (0.7%) |
| Missing | | | | | | | | | | | | |
| ≦ULN | 57/59 | (96.6%) | 49/57 | (86.0%) | 106/116 | (91.4%) | 159/186 | (85.5%) | 148/171 | (86.5%) | 307/357 | (86.0%) |
| >ULN-<20 ng/L | 2/59 | (3.4%) | 6/57 | (10.5%) | 8/116 | (6.9%) | 23/186 | (12.4%) | 20/171 | (11.7%) | 43/357 | (12.0%) |
| ≧20 ng/L-<50 ng/L | 0/59 | | 2/57 | (3.5%) | 2/116 | (1.7%) | 3/186 | (1.6%) | 2/171 | (1.2%) | 5/357 | (1.4%) |
| ≧50 ng/L | 0/59 | | 0/57 | | 0/116 | | 1/186 | (0.5%) | 1/171 | (0.6%) | 2/357 | (0.6%) |
| ≦ULN | | | | | | | | | | | | |
| ≦ULN | 14/16 | (87.5%) | 21/21 | (100%) | 35/37 | (94.6%) | 39/42 | (92.9%) | 43/47 | (91.5%) | 82/89 | (92.1%) |
| >ULN-<20 ng/L | 2/16 | (12.5%) | 0/21 | | 2/37 | (5.4%) | 3/42 | (7.1%) | 4/47 | (8.5%) | 7/89 | (7.9%) |
| ≧20 ng/L-<50 ng/L | 0/16 | | 0/21 | | 0/37 | | 0/42 | | 0/47 | | 0/89 | |
| ≧50 ng/L | 0/16 | | 0/21 | | 0/37 | | 0/42 | | 0/47 | | 0/89 | |
| >ULN-<20 ng/L | | | | | | | | | | | | |
| ≦ULN | 0/4 | | 0/1 | | 0/5 | | 0/4 | | 0/6 | | 0/10 | |
| >ULN-<20 ng/L | 2/4 | (50.0%) | 1/1 | (100%) | 3/5 | (60.0%) | 4/4 | (100%) | 4/6 | (66.7%) | 8/10 | (80.0%) |
| ≧20 ng/L-<50 ng/L | 2/4 | (50.0%) | 0/1 | | 2/5 | (40.0%) | 0/4 | | 2/6 | (33.3%) | 2/10 | (20.0%) |
| ≧50 ng/L | 0/4 | | 0/1 | | 0/5 | | 0/4 | | 0/6 | | 0/10 | |
| ≧20 ng/L-<50 ng/L | | | | | | | | | | | | |
| ≦ULN | 0/0 | | 0/2 | | 0/2 | | 0/0 | | 0/2 | | 0/2 | |
| >ULN-<20 ng/L | 0/0 | | 1/2 | (50.0%) | 1/2 | (50.0%) | 0/0 | | 0/2 | | 0/2 | |
| ≧20 ng/L-<50 ng/L | 0/0 | | 1/2 | (50.0%) | 1/2 | (50.0%) | 0/0 | | 2/2 | (100%) | 2/2 | (100%) |
| ≧50 ng/L | 0/0 | | 0/2 | | 0/2 | | 0/0 | | 0/2 | | 0/2 | |
| ≧50 ng/L | | | | | | | | | | | | |
| ≦ULN | 0/0 | | 0/0 | | 0/0 | | 0/0 | | 0/1 | | 0/1 | |
| >ULN-<20 ng/L | 0/0 | | 0/0 | | 0/0 | | 0/0 | | 0/1 | | 0/1 | |
| ≧20 ng/L-<50 ng/L | 0/0 | | 0/0 | | 0/0 | | 0/0 | | 0/1 | | 0/1 | |
| ≧50 ng/L | 0/0 | | 0/0 | | 0/0 | | 0/0 | | 1/1 | (100%) | 1/1 | (100%) |

ULN = Upper limit of normal.
On-treatment period of the whole study = the time from the first dose of double-blind study medication up to 3 days after the last dose administration.
*Regardless of baseline.
Note:
The numerator represents the number of patients who were in the pre-specified categories at post-baseline in each baseline category. The denominator (/N1) is the number of patients who had that parameter assessed post-baseline by baseline status. A patient is counted only in the worst category.

### 7 APPENDIX

TABLE 30
Number (%) of patients by dose at the end of titration - Safety population

| | Placebo | | | Lixisenatide | | |
|---|---|---|---|---|---|---|
| | Morning | Evening | | Morning | Evening | |
| | Injection | Injection | Combined | Injection | Injection | Combined |
| Dose at the end of titration | (N = 85) | (N = 85) | (N = 170) | (N = 255) | (N = 255) | (N = 510) |
| 10 μg | 1 (1.2%) | 1 (1.2%) | 2 (1.2%) | 4 (1.6%) | 3 (1.2%) | 7 (1.4%) |
| 15 μg | 5 (5.9%) | 0 | 5 (2.9%) | 14 (5.5%) | 16 (6.3%) | 30 (5.9%) |
| 20 μg | 79 (92.9%) | 84 (98.8%) | 163 (95.9%) | 237 (92.9%) | 236 (92.5%) | 473 (92.7%) |

Dose = Dose of active drug or volume-matched placebo.
The scheduled visit for end of titration per protocol would be Visit 5/Week 2.
Note:
Percents are calculated using the number of safety patients as the denominator.

TABLE 31
Mean change in HbA1c (%) from baseline by visit - mITT population

| Treatment | Observed data | | | | | | | Change from baseline | | | | | | |
|---|---|---|---|---|---|---|---|---|---|---|---|---|---|---|
| Time point | N | Mean | SD | SE | Median | Min | Max | N | Mean | SD | SE | Median | Min | Max |
| Placebo Combined | | | | | | | | | | | | | | |
| (N = 170) | | | | | | | | | | | | | | |
| Screening | 170 | 8.15 | 0.86 | 0.066 | 8.00 | 7.0 | 10.0 | | | | | | | |
| Baseline | 170 | 8.06 | 0.90 | 0.069 | 7.85 | 6.4 | 10.5 | | | | | | | |
| Week 8 | 157 | 7.64 | 0.87 | 0.069 | 7.60 | 5.0 | 9.9 | 157 | −0.37 | 0.72 | 0.058 | −0.30 | −4.1 | 1.7 |
| Week 12 | 154 | 7.65 | 1.01 | 0.082 | 7.55 | 5.2 | 13.0 | 154 | −0.38 | 0.95 | 0.076 | −0.30 | −4.1 | 5.0 |
| Week 24 | 139 | 7.54 | 1.01 | 0.085 | 7.40 | 5.2 | 13.8 | 139 | −0.41 | 1.08 | 0.091 | −0.40 | −3.6 | 5.8 |
| Week 24 | 164 | 7.67 | 1.08 | 0.084 | 7.55 | 5.2 | 13.8 | 164 | −0.35 | 1.06 | 0.083 | −0.30 | −3.6 | 5.8 |
| (LOCF) | | | | | | | | | | | | | | |
| Week 36 | 125 | 7.40 | 0.89 | 0.079 | 7.40 | 5.7 | 11.5 | 125 | −0.49 | 1.01 | 0.091 | −0.40 | −2.8 | 3.5 |
| Week 44 | 114 | 7.33 | 0.91 | 0.085 | 7.30 | 5.4 | 12.2 | 114 | −0.54 | 1.10 | 0.103 | −0.50 | −3.3 | 4.2 |
| Week 52 | 108 | 7.29 | 0.83 | 0.080 | 7.20 | 5.7 | 10.7 | 108 | −0.56 | 1.00 | 0.096 | −0.50 | −3.1 | 3.4 |
| Week 60 | 100 | 7.31 | 0.83 | 0.083 | 7.20 | 5.7 | 9.5 | 100 | −0.50 | 0.95 | 0.095 | −0.45 | −2.8 | 1.9 |
| Week 68 | 93 | 7.34 | 0.92 | 0.095 | 7.20 | 5.4 | 10.2 | 93 | −0.49 | 1.03 | 0.107 | −0.60 | −2.7 | 2.7 |
| Week 76 | 88 | 7.34 | 0.87 | 0.093 | 7.25 | 5.9 | 10.1 | 88 | −0.42 | 0.99 | 0.106 | −0.50 | −2.7 | 3.1 |
| Week 84 | 51 | 7.34 | 0.89 | 0.124 | 7.20 | 5.5 | 9.5 | 51 | −0.42 | 0.92 | 0.128 | −0.40 | −2.5 | 2.0 |
| Week 92 | 34 | 7.29 | 0.73 | 0.125 | 7.30 | 5.4 | 9.2 | 34 | −0.61 | 0.94 | 0.161 | −0.60 | −2.9 | 1.6 |
| Week 100 | 20 | 7.50 | 1.00 | 0.223 | 7.35 | 5.6 | 10.3 | 20 | −0.48 | 1.12 | 0.250 | −0.55 | −2.5 | 2.7 |
| Week 108 | 6 | 7.98 | 1.06 | 0.431 | 7.60 | 7.0 | 9.7 | 6 | −0.58 | 1.42 | 0.582 | −0.65 | −2.5 | 1.7 |
| Week 116 | 2 | 8.60 | 1.84 | 1.300 | 8.60 | 7.3 | 9.9 | 2 | 0.30 | 0.57 | 0.400 | 0.30 | −0.1 | 0.7 |
| Week 124 | 1 | 7.40 | NC | NC | 7.40 | 7.4 | 7.4 | 1 | 0.00 | NC | NC | 0.00 | 0.0 | 0.0 |
| Last on- | 164 | 7.90 | 1.09 | 0.085 | 7.80 | 5.6 | 11.6 | 164 | −0.12 | 1.03 | 0.080 | −0.10 | −2.6 | 3.6 |
| treatment | | | | | | | | | | | | | | |
| value | | | | | | | | | | | | | | |
| Lixisenatide | | | | | | | | | | | | | | |
| Morning | | | | | | | | | | | | | | |
| Injection | | | | | | | | | | | | | | |
| (N = 255) | | | | | | | | | | | | | | |
| Screening | 255 | 8.15 | 0.84 | 0.053 | 8.00 | 5.7 | 10.0 | | | | | | | |
| Baseline | 255 | 8.05 | 0.90 | 0.056 | 7.90 | 5.3 | 12.0 | | | | | | | |
| Week 8 | 239 | 7.33 | 0.85 | 0.055 | 7.30 | 4.7 | 11.2 | 239 | −0.73 | 0.72 | 0.047 | −0.70 | −2.7 | 2.7 |
| Week 12 | 237 | 7.23 | 0.89 | 0.058 | 7.10 | 4.5 | 10.3 | 237 | −0.84 | 0.78 | 0.050 | −0.80 | −2.9 | 1.2 |
| Week 24 | 224 | 7.17 | 0.97 | 0.065 | 7.00 | 5.1 | 11.0 | 224 | −0.88 | 0.90 | 0.060 | −0.90 | −3.3 | 2.9 |
| Week 24 | 244 | 7.24 | 0.99 | 0.063 | 7.10 | 5.1 | 11.0 | 244 | −0.83 | 0.91 | 0.058 | −0.90 | −3.3 | 2.9 |
| (LOCF) | | | | | | | | | | | | | | |
| Week 36 | 205 | 7.03 | 0.79 | 0.055 | 6.90 | 5.2 | 9.9 | 205 | −0.98 | 0.94 | 0.066 | −1.00 | −5.5 | 1.2 |
| Week 44 | 187 | 7.02 | 0.83 | 0.061 | 6.90 | 5.3 | 10.3 | 187 | −0.99 | 0.91 | 0.067 | −1.00 | −3.7 | 1.6 |
| Week 52 | 180 | 6.96 | 0.86 | 0.064 | 6.90 | 5.0 | 10.7 | 180 | −1.06 | 0.97 | 0.072 | −1.00 | −5.5 | 1.6 |
| Week 60 | 168 | 6.99 | 0.84 | 0.065 | 7.00 | 5.0 | 10.2 | 168 | −1.01 | 0.99 | 0.076 | −0.90 | −4.4 | 2.5 |
| Week 68 | 162 | 7.02 | 0.93 | 0.073 | 6.95 | 5.1 | 11.0 | 162 | −0.96 | 1.05 | 0.083 | −1.00 | −3.7 | 2.6 |
| Week 76 | 153 | 7.05 | 0.86 | 0.069 | 7.00 | 5.3 | 10.8 | 153 | −0.91 | 0.99 | 0.080 | −0.90 | −3.5 | 2.4 |
| Week 84 | 92 | 7.07 | 0.99 | 0.103 | 6.95 | 5.2 | 12.0 | 92 | −0.82 | 1.13 | 0.118 | −0.80 | −3.3 | 3.6 |
| Week 92 | 70 | 7.06 | 0.87 | 0.104 | 6.95 | 5.1 | 11.3 | 70 | −0.86 | 1.10 | 0.131 | −0.80 | −3.1 | 2.9 |
| Week 100 | 34 | 6.89 | 0.60 | 0.103 | 6.85 | 5.6 | 8.1 | 34 | −1.13 | 0.94 | 0.160 | −1.25 | −2.8 | 0.9 |
| Week 108 | 16 | 7.21 | 0.79 | 0.197 | 7.00 | 6.2 | 8.7 | 16 | −0.78 | 1.21 | 0.303 | −0.50 | −3.0 | 0.9 |
| Week 116 | 4 | 7.30 | 0.74 | 0.372 | 7.15 | 6.6 | 8.3 | 4 | −0.97 | 1.27 | 0.633 | −1.30 | −2.1 | 0.8 |
| Week 124 | 1 | 6.60 | NC | NC | 6.60 | 6.6 | 6.6 | 1 | −1.30 | NC | NC | −1.30 | −1.3 | −1.3 |
| Last on- | 244 | 7.50 | 1.07 | 0.069 | 7.30 | 5.1 | 11.3 | 244 | −0.58 | 1.09 | 0.069 | −0.60 | −5.5 | 3.6 |
| treatment | | | | | | | | | | | | | | |
| value | | | | | | | | | | | | | | |
| Lixisenatide | | | | | | | | | | | | | | |
| Evening Injection | | | | | | | | | | | | | | |
| (N = 255) | | | | | | | | | | | | | | |
| Screening | 255 | 8.22 | 0.86 | 0.054 | 8.00 | 7.0 | 10.0 | | | | | | | |
| Baseline | 255 | 8.08 | 0.88 | 0.055 | 8.00 | 6.5 | 10.2 | | | | | | | |
| Week 8 | 232 | 7.38 | 1.01 | 0.066 | 7.20 | 5.3 | 11.5 | 232 | −0.66 | 0.72 | 0.047 | −0.70 | −2.9 | 2.3 |
| Week 12 | 226 | 7.26 | 1.00 | 0.067 | 7.10 | 5.2 | 11.7 | 226 | −0.77 | 0.77 | 0.051 | −0.70 | −3.0 | 2.5 |
| Week 24 | 212 | 7.23 | 0.92 | 0.063 | 7.05 | 5.4 | 10.7 | 212 | −0.79 | 0.79 | 0.054 | −0.75 | −3.0 | 1.5 |
| Week 24 | 239 | 7.34 | 1.04 | 0.067 | 7.10 | 5.4 | 11.7 | 239 | −0.73 | 0.84 | 0.055 | −0.70 | −3.0 | 2.5 |
| (LOCF) | | | | | | | | | | | | | | |
| Week 36 | 178 | 7.11 | 0.89 | 0.067 | 7.00 | 5.2 | 11.2 | 178 | −0.84 | 0.85 | 0.063 | −0.80 | −3.6 | 1.9 |
| Week 44 | 168 | 7.03 | 0.84 | 0.065 | 6.90 | 5.2 | 10.2 | 168 | −0.85 | 0.89 | 0.069 | −0.80 | −3.9 | 2.1 |
| Week 52 | 165 | 7.02 | 0.79 | 0.062 | 7.00 | 5.3 | 10.5 | 165 | −0.84 | 0.90 | 0.070 | −0.70 | −4.2 | 2.4 |
| Week 60 | 158 | 7.02 | 0.77 | 0.061 | 7.00 | 5.3 | 10.0 | 158 | −0.84 | 0.87 | 0.069 | −0.80 | −3.9 | 1.9 |
| Week 68 | 153 | 7.02 | 0.76 | 0.061 | 7.00 | 5.3 | 9.4 | 153 | −0.82 | 0.90 | 0.073 | −0.70 | −4.3 | 1.5 |
| Week 76 | 150 | 7.13 | 0.83 | 0.068 | 7.00 | 5.2 | 9.9 | 150 | −0.70 | 0.95 | 0.077 | −0.60 | −4.2 | 2.0 |
| Week 84 | 90 | 7.16 | 0.85 | 0.090 | 7.10 | 5.0 | 10.4 | 90 | −0.56 | 0.81 | 0.086 | −0.50 | −2.9 | 1.7 |
| Week 92 | 64 | 7.20 | 0.88 | 0.110 | 7.05 | 5.3 | 10.1 | 64 | −0.56 | 0.84 | 0.105 | −0.60 | −2.2 | 2.0 |
| Week 100 | 29 | 7.13 | 0.85 | 0.158 | 6.90 | 5.6 | 8.9 | 29 | −0.59 | 0.71 | 0.132 | −0.50 | −1.8 | 0.9 |
| Week 108 | 12 | 7.42 | 0.89 | 0.258 | 7.35 | 5.9 | 8.8 | 12 | −0.37 | 0.72 | 0.209 | −0.35 | −1.4 | 1.1 |
| Week 116 | 5 | 7.64 | 1.02 | 0.455 | 7.80 | 6.5 | 8.8 | 5 | −0.18 | 1.21 | 0.542 | 0.00 | −1.5 | 1.5 |
| Last on- | 239 | 7.65 | 1.15 | 0.074 | 7.50 | 5.4 | 12.4 | 239 | −0.42 | 1.03 | 0.067 | −0.40 | −4.2 | 4.4 |
| treatment | | | | | | | | | | | | | | |
| value | | | | | | | | | | | | | | |

LOCF = Last observation carried forward.
The analysis excluded measurements obtained after the introduction of rescue medication and/or after the treatment cessation plus 3 days.
For Week 24 (LOCF), the analysis included measurements obtained up to 3 days after the last dose of the double-blind investigational product injection on or before Visit 12 (Week 24), or Day 169 if Visit 12 (Week 24) is not available.

TABLE 32
Number (%) of patients experiencing common TEAE(s) by primary SOC, HLGT, HLT and PT during the on-treatment period of the whole
study - Safety population

| PRIMARY SYSTEM ORGAN CLASS | Placebo | | | | | | Lixisenatide | | | | | |
|---|---|---|---|---|---|---|---|---|---|---|---|---|
| HLGT: High Level Group Term | Morning | | Evening | | | | Morning | | Evening | | | |
| HLT: High Level Term | Injection | | Injection | | Combined | | Injection | | Injection | | Combined | |
| Preferred Term n (%) | (N = 85) | | (N = 85) | | (N = 170) | | (N = 255) | | (N = 255) | | (N = 510) | |
| Any class | 60 | (70.6%) | 68 | (80.0%) | 128 | (75.3%) | 216 | (84.7%) | 213 | (83.5%) | 429 | (84.1%) |
| INFECTIONS AND INFESTATIONS | 40 | (47.1%) | 47 | (55.3%) | 87 | (51.2%) | 140 | (54.9%) | 98 | (38.4%) | 238 | (46.7%) |
| HLGT: Fungal infectious disorders | 0 | | 3 | (3.5%) | 3 | (1.8%) | 8 | (3.1%) | 6 | (2.4%) | 14 | (2.7%) |
| HLT: Tinea infections | 0 | | 0 | | 0 | | 5 | (2.0%) | 1 | (0.4%) | 6 | (1.2%) |
| Tinea pedis | 0 | | 0 | | 0 | | 5 | (2.0%) | 1 | (0.4%) | 6 | (1.2%) |
| HLGT: Infections - pathogen unspecified | 34 | (40.0%) | 39 | (45.9%) | 73 | (42.9%) | 124 | (48.6%) | 78 | (30.6%) | 202 | (39.6%) |
| HLT: Abdominal and gastrointestinal | 4 | (4.7%) | 5 | (5.9%) | 9 | (5.3%) | 18 | (7.1%) | 14 | (5.5%) | 32 | (6.3%) |
| infections | | | | | | | | | | | | |
| Gastroenteritis | 4 | (4.7%) | 4 | (4.7%) | 8 | (4.7%) | 14 | (5.5%) | 12 | (4.7%) | 26 | (5.1%) |
| HLT: Dental and oral soft tissue infections | 3 | (3.5%) | 4 | (4.7%) | 7 | (4.1%) | 8 | (3.1%) | 7 | (2.7%) | 15 | (2.9%) |
| Tooth abscess | 2 | (2.4%) | 0 | | 2 | (1.2%) | 2 | (0.8%) | 4 | (1.6%) | 6 | (1.2%) |
| Tooth infection | 0 | | 3 | (3.5%) | 3 | (1.8%) | 4 | (1.6%) | 3 | (1.2%) | 7 | (1.4%) |
| HLT: Ear infections | 0 | | 0 | | 0 | | 4 | (1.6%) | 2 | (0.8%) | 6 | (1.2%) |
| Ear infection | 0 | | 0 | | 0 | | 3 | (1.2%) | 2 | (0.8%) | 5 | (1.0%) |
| HLT: Infections NEC | 2 | (2.4%) | 2 | (2.4%) | 4 | (2.4%) | 10 | (3.9%) | 10 | (3.9%) | 20 | (3.9%) |
| Localised infection | 1 | (1.2%) | 1 | (1.2%) | 2 | (1.2%) | 2 | (0.8%) | 3 | (1.2%) | 5 | (1.0%) |
| Respiratory tract infection | 1 | (1.2%) | 1 | (1.2%) | 2 | (1.2%) | 4 | (1.6%) | 4 | (1.6%) | 8 | (1.6%) |
| Wound infection | 0 | | 0 | | 0 | | 0 | | 3 | (1.2%) | 3 | (0.6%) |
| HLT: Lower respiratory tract and lung | 8 | (9.4%) | 10 | (11.8%) | 18 | (10.6%) | 26 | (10.2%) | 10 | (3.9%) | 36 | (7.1%) |
| infections | | | | | | | | | | | | |
| Bronchitis | 8 | (9.4%) | 6 | (7.1%) | 14 | (8.2%) | 22 | (8.6%) | 6 | (2.4%) | 28 | (5.5%) |
| Lower respiratory tract infection | 0 | | 1 | (1.2%) | 1 | (0.6%) | 3 | (1.2%) | 0 | | 3 | (0.6%) |
| Pneumonia | 0 | | 4 | (4.7%) | 4 | (2.4%) | 2 | (0.8%) | 4 | (1.6%) | 6 | (1.2%) |
| HLT: Upper respiratory tract infections | 20 | (23.5%) | 28 | (32.9%) | 48 | (28.2%) | 74 | (29.0%) | 49 | (19.2%) | 123 | (24.1%) |
| Acute tonsillitis | 1 | (1.2%) | 1 | (1.2%) | 2 | (1.2%) | 3 | (1.2%) | 0 | | 3 | (0.6%) |
| Nasopharyngitis | 11 | (12.9%) | 15 | (17.6%) | 26 | (15.3%) | 38 | (14.9%) | 20 | (7.8%) | 58 | (11.4%) |
| Pharyngitis | 2 | (2.4%) | 3 | (3.5%) | 5 | (2.9%) | 6 | (2.4%) | 16 | (6.3%) | 22 | (4.3%) |
| Pharyngotonsillitis | 1 | (1.2%) | 3 | (3.5%) | 4 | (2.4%) | 3 | (1.2%) | 3 | (1.2%) | 6 | (1.2%) |
| Rhinitis | 1 | (1.2%) | 1 | (1.2%) | 2 | (1.2%) | 0 | | 1 | (0.4%) | 1 | (0.2%) |
| Sinusitis | 0 | | 0 | | 0 | | 8 | (3.1%) | 5 | (2.0%) | 13 | (2.5%) |
| Tonsillitis | 0 | | 1 | (1.2%) | 1 | (0.6%) | 0 | | 3 | (1.2%) | 3 | (0.6%) |
| Upper respiratory tract infection | 6 | (7.1%) | 10 | (11.8%) | 16 | (9.4%) | 22 | (8.6%) | 15 | (5.9%) | 37 | (7.3%) |
| HLT: Urinary tract infections | 2 | (2.4%) | 6 | (7.1%) | 8 | (4.7%) | 18 | (7.1%) | 14 | (5.5%) | 32 | (6.3%) |
| Cystitis | 0 | | 1 | (1.2%) | 1 | (0.6%) | 4 | (1.6%) | 4 | (1.6%) | 8 | (1.6%) |
| Urinary tract infection | 2 | (2.4%) | 5 | (5.9%) | 7 | (4.1%) | 15 | (5.9%) | 10 | (3.9%) | 25 | (4.9%) |
| HLGT: Viral infectious disorders | 8 | (9.4%) | 17 | (20.0%) | 25 | (14.7%) | 49 | (19.2%) | 40 | (15.7%) | 89 | (17.5%) |
| HLT: Flaviviral infections | 0 | | 0 | | 0 | | 3 | (1.2%) | 0 | | 3 | (0.6%) |
| Dengue fever | 0 | | 0 | | 0 | | 3 | (1.2%) | 0 | | 3 | (0.6%) |
| HLT: Herpes viral infections | 1 | (1.2%) | 1 | (1.2%) | 2 | (1.2%) | 4 | (1.6%) | 2 | (0.8%) | 6 | (1.2%) |
| Herpes zoster | 0 | | 0 | | 0 | | 3 | (1.2%) | 1 | (0.4%) | 4 | (0.8%) |
| Oral herpes | 1 | (1.2%) | 1 | (1.2%) | 2 | (1.2%) | 1 | (0.4%) | 1 | (0.4%) | 2 | (0.4%) |
| HLT: Influenza viral infections | 5 | (5.9%) | 9 | (10.6%) | 14 | (8.2%) | 30 | (11.8%) | 28 | (11.0%) | 58 | (11.4%) |
| Influenza | 5 | (5.9%) | 9 | (10.6%) | 14 | (8.2%) | 30 | (11.8%) | 28 | (11.0%) | 58 | (11.4%) |
| HLT: Viral infections NEC | 2 | (2.4%) | 9 | (10.6%) | 11 | (6.5%) | 13 | (5.1%) | 13 | (5.1%) | 26 | (5.1%) |
| Gastroenteritis viral | 0 | | 3 | (3.5%) | 3 | (1.8%) | 3 | (1.2%) | 4 | (1.6%) | 7 | (1.4%) |
| Respiratory tract infection viral | 0 | | 1 | (1.2%) | 1 | (0.6%) | 1 | (0.4%) | 3 | (1.2%) | 4 | (0.8%) |
| Viral infection | 1 | (1.2%) | 0 | | 1 | (0.6%) | 5 | (2.0%) | 2 | (0.8%) | 7 | (1.4%) |
| Viral upper respiratory tract infection | 1 | (1.2%) | 4 | (4.7%) | 5 | (2.9%) | 2 | (0.8%) | 2 | (0.8%) | 4 | (0.8%) |
| NEOPLASMS BENIGN, MALIGNANT AND | 2 | (2.4%) | 3 | (3.5%) | 5 | (2.9%) | 9 | (3.5%) | 4 | (1.6%) | 13 | (2.5%) |
| UNSPECIFIED (INCL CYSTS AND POLYPS) | | | | | | | | | | | | |
| HLGT: Endocrine neoplasms malignant and | 0 | | 0 | | 0 | | 4 | (1.6%) | 0 | | 4 | (0.8%) |
| unspecified | | | | | | | | | | | | |
| HLT: Endocrine neoplasms malignant and | 0 | | 0 | | 0 | | 4 | (1.6%) | 0 | | 4 | (0.8%) |
| unspecified NEC | | | | | | | | | | | | |
| Thyroid neoplasm | 0 | | 0 | | 0 | | 4 | (1.6%) | 0 | | 4 | (0.8%) |
| BLOOD AND LYMPHATIC SYSTEM | 3 | (3.5%) | 3 | (3.5%) | 6 | (3.5%) | 4 | (1.6%) | 8 | (3.1%) | 12 | (2.4%) |
| DISORDERS | | | | | | | | | | | | |
| HLGT: Anaemias nonhaemolytic and marrow | 2 | (2.4%) | 1 | (1.2%) | 3 | (1.8%) | 1 | (0.4%) | 3 | (1.2%) | 4 | (0.8%) |
| depression | | | | | | | | | | | | |
| HLT: Anaemias NEC | 1 | (1.2%) | 1 | (1.2%) | 2 | (1.2%) | 1 | (0.4%) | 3 | (1.2%) | 4 | (0.8%) |
| Anaemia | 0 | | 1 | (1.2%) | 1 | (0.6%) | 1 | (0.4%) | 3 | (1.2%) | 4 | (0.8%) |
| HLGT: White blood cell disorders | 0 | | 2 | (2.4%) | 2 | (1.2%) | 3 | (1.2%) | 3 | (1.2%) | 6 | (1.2%) |
| HLT: Eosinophilic disorders | 0 | | 2 | (2.4%) | 2 | (1.2%) | 1 | (0.4%) | 1 | (0.4%) | 2 | (0.4%) |
| Eosinophilia | 0 | | 2 | (2.4%) | 2 | (1.2%) | 1 | (0.4%) | 1 | (0.4%) | 2 | (0.4%) |
| IMMUNE SYSTEM DISORDERS | 1 | (1.2%) | 1 | (1.2%) | 2 | (1.2%) | 5 | (2.0%) | 3 | (1.2%) | 8 | (1.6%) |
| HLGT: Allergic conditions | 1 | (1.2%) | 1 | (1.2%) | 2 | (1.2%) | 5 | (2.0%) | 3 | (1.2%) | 8 | (1.6%) |
| HLT: Atopic disorders | 0 | | 1 | (1.2%) | 1 | (0.6%) | 3 | (1.2%) | 1 | (0.4%) | 4 | (0.8%) |
| Seasonal allergy | 0 | | 1 | (1.2%) | 1 | (0.6%) | 3 | (1.2%) | 1 | (0.4%) | 4 | (0.8%) |
| ENDOCRINE DISORDERS | 2 | (2.4%) | 1 | (1.2%) | 3 | (1.8%) | 4 | (1.6%) | 2 | (0.8%) | 6 | (1.2%) |
| HLGT: Thyroid gland disorders | 2 | (2.4%) | 1 | (1.2%) | 3 | (1.8%) | 4 | (1.6%) | 2 | (0.8%) | 6 | (1.2%) |
| HLT: Thyroid disorders NEC | 1 | (1.2%) | 1 | (1.2%) | 2 | (1.2%) | 1 | (0.4%) | 0 | | 1 | (0.2%) |
| Goitre | 1 | (1.2%) | 1 | (1.2%) | 2 | (1.2%) | 1 | (0.4%) | 0 | | 1 | (0.2%) |
| METABOLISM AND NUTRITION DISORDERS | 4 | (4.7%) | 15 | (17.6%) | 19 | (11.2%) | 46 | (18.0%) | 54 | (21.2%) | 100 | (19.6%) |
| HLGT: Appetite and general nutritional disorders | 0 | | 1 | (1.2%) | 1 | (0.6%) | 11 | (4.3%) | 10 | (3.9%) | 21 | (4.1%) |
| HLT: Appetite disorders | 0 | | 1 | (1.2%) | 1 | (0.6%) | 11 | (4.3%) | 9 | (3.5%) | 20 | (3.9%) |
| Decreased appetite | 0 | | 1 | (1.2%) | 1 | (0.6%) | 10 | (3.9%) | 8 | (3.1%) | 18 | (3.5%) |
| HLGT: Glucose metabolism disorders (incl | 1 | (1.2%) | 5 | (5.9%) | 6 | (3.5%) | 25 | (9.8%) | 32 | (12.5%) | 57 | (11.2%) |
| diabetes mellitus) | | | | | | | | | | | | |
| HLT: Hyperglycaemic conditions NEC | 1 | (1.2%) | 0 | | 1 | (0.6%) | 1 | (0.4%) | 3 | (1.2%) | 4 | (0.8%) |
| Hyperglycaemia | 1 | (1.2%) | 0 | | 1 | (0.6%) | 1 | (0.4%) | 3 | (1.2%) | 4 | (0.8%) |
| HLT: Hypoglycaemic conditions NEC | 0 | | 5 | (5.9%) | 5 | (2.9%) | 23 | (9.0%) | 28 | (11.0%) | 51 | (10.0%) |
| Hypoglycaemia | 0 | | 5 | (5.9%) | 5 | (2.9%) | 23 | (9.0%) | 28 | (11.0%) | 51 | (10.0%) |
| HLGT: Lipid metabolism disorders | 2 | (2.4%) | 4 | (4.7%) | 6 | (3.5%) | 11 | (4.3%) | 8 | (3.1%) | 19 | (3.7%) |
| HLT: Elevated cholesterol | 0 | | 0 | | 0 | | 2 | (0.8%) | 3 | (1.2%) | 5 | (1.0%) |
| Hypercholesterolaemia | 0 | | 0 | | 0 | | 2 | (0.8%) | 3 | (1.2%) | 5 | (1.0%) |
| HLT: Elevated triglycerides | 2 | (2.4%) | 4 | (4.7%) | 6 | (3.5%) | 8 | (3.1%) | 4 | (1.6%) | 12 | (2.4%) |
| Hypertriglyceridaemia | 2 | (2.4%) | 4 | (4.7%) | 6 | (3.5%) | 8 | (3.1%) | 4 | (1.6%) | 12 | (2.4%) |
| HLGT: Purine and pyrimidine metabolism | 1 | (1.2%) | 2 | (2.4%) | 3 | (1.8%) | 2 | (0.8%) | 7 | (2.7%) | 9 | (1.8%) |
| disorders | | | | | | | | | | | | |
| HLT: Purine metabolism disorders NEC | 1 | (1.2%) | 2 | (2.4%) | 3 | (1.8%) | 2 | (0.8%) | 7 | (2.7%) | 9 | (1.8%) |
| Gout | 1 | (1.2%) | 1 | (1.2%) | 2 | (1.2%) | 1 | (0.4%) | 2 | (0.8%) | 3 | (0.6%) |
| Hyperuricaemia | 0 | | 1 | (1.2%) | 1 | (0.6%) | 1 | (0.4%) | 5 | (2.0%) | 6 | (1.2%) |
| PSYCHIATRIC DISORDERS | 4 | (4.7%) | 8 | (9.4%) | 12 | (7.1%) | 34 | (13.3%) | 28 | (11.0%) | 62 | (12.2%) |
| HLGT: Anxiety disorders and symptoms | 2 | (2.4%) | 6 | (7.1%) | 8 | (4.7%) | 16 | (6.3%) | 10 | (3.9%) | 26 | (5.1%) |
| HLT: Anxiety symptoms | 2 | (2.4%) | 4 | (4.7%) | 6 | (3.5%) | 15 | (5.9%) | 9 | (3.5%) | 24 | (4.7%) |
| Anxiety | 2 | (2.4%) | 4 | (4.7%) | 6 | (3.5%) | 12 | (4.7%) | 9 | (3.5%) | 21 | (4.1%) |
| Nervousness | 0 | | 0 | | 0 | | 4 | (1.6%) | 0 | | 4 | (0.8%) |
| HLT: Panic attacks and disorders | 0 | | 2 | (2.4%) | 2 | (1.2%) | 1 | (0.4%) | 0 | | 1 | (0.2%) |
| Panic attack | 0 | | 2 | (2.4%) | 2 | (1.2%) | 0 | | 0 | | 0 | |
| HLGT: Depressed mood disorders and | 1 | (1.2%) | 2 | (2.4%) | 3 | (1.8%) | 12 | (4.7%) | 9 | (3.5%) | 21 | (4.1%) |
| disturbances | | | | | | | | | | | | |
| HLT: Depressive disorders | 1 | (1.2%) | 2 | (2.4%) | 3 | (1.8%) | 11 | (4.3%) | 9 | (3.5%) | 20 | (3.9%) |
| Depression | 1 | (1.2%) | 2 | (2.4%) | 3 | (1.8%) | 11 | (4.3%) | 9 | (3.5%) | 20 | (3.9%) |
| HLGT: Sleep disorders and disturbances | 1 | (1.2%) | 0 | | 1 | (0.6%) | 4 | (1.6%) | 13 | (5.1%) | 17 | (3.3%) |
| HLT: Disturbances in initiating and | 1 | (1.2%) | 0 | | 1 | (0.6%) | 3 | (1.2%) | 13 | (5.1%) | 16 | (3.1%) |
| maintaining sleep | | | | | | | | | | | | |
| Insomnia | 1 | (1.2%) | 0 | | 1 | (0.6%) | 3 | (1.2%) | 12 | (4.7%) | 15 | (2.9%) |
| NERVOUS SYSTEM DISORDERS | 13 | (15.3%) | 30 | (35.3%) | 43 | (25.3%) | 87 | (34.1%) | 74 | (29.0%) | 161 | (31.6%) |
| HLGT: Headaches | 8 | (9.4%) | 20 | (23.5%) | 28 | (16.5%) | 49 | (19.2%) | 44 | (17.3%) | 93 | (18.2%) |
| HLT: Headaches NEC | 8 | (9.4%) | 20 | (23.5%) | 28 | (16.5%) | 49 | (19.2%) | 43 | (16.9%) | 92 | (18.0%) |
| Headache | 8 | (9.4%) | 20 | (23.5%) | 28 | (16.5%) | 49 | (19.2%) | 42 | (16.5%) | 91 | (17.8%) |
| HLT: Migraine headaches | 0 | | 0 | | 0 | | 3 | (1.2%) | 2 | (0.8%) | 5 | (1.0%) |
| Migraine | 0 | | 0 | | 0 | | 3 | (1.2%) | 2 | (0.8%) | 5 | (1.0%) |
| HLGT: Movement disorders (incl parkinsonism) | 0 | | 0 | | 0 | | 10 | (3.9%) | 7 | (2.7%) | 17 | (3.3%) |
| HLT: Tremor (excl congenital) | 0 | | 0 | | 0 | | 9 | (3.5%) | 6 | (2.4%) | 15 | (2.9%) |
| Tremor | 0 | | 0 | | 0 | | 9 | (3.5%) | 6 | (2.4%) | 15 | (2.9%) |
| HLGT: Neurological disorders NEC | 6 | (7.1%) | 12 | (14.1%) | 18 | (10.6%) | 34 | (13.3%) | 26 | (10.2%) | 60 | (11.8%) |
| HLT: Disturbances in consciousness NEC | 1 | (1.2%) | 4 | (4.7%) | 5 | (2.9%) | 7 | (2.7%) | 5 | (2.0%) | 12 | (2.4%) |
| Somnolence | 1 | (1.2%) | 2 | (2.4%) | 3 | (1.8%) | 3 | (1.2%) | 3 | (1.2%) | 6 | (1.2%) |
| HLT: Neurological signs and symptoms NEC | 5 | (5.9%) | 6 | (7.1%) | 11 | (6.5%) | 18 | (7.1%) | 14 | (5.5%) | 32 | (6.3%) |
| Dizziness | 5 | (5.9%) | 6 | (7.1%) | 11 | (6.5%) | 18 | (7.1%) | 14 | (5.5%) | 32 | (6.3%) |
| HLT: Paraesthesias and dysaesthesias | 0 | | 4 | (4.7%) | 4 | (2.4%) | 7 | (2.7%) | 5 | (2.0%) | 12 | (2.4%) |
| Paraesthesia | 0 | | 4 | (4.7%) | 4 | (2.4%) | 5 | (2.0%) | 4 | (1.6%) | 9 | (1.8%) |
| HLT: Sensory abnormalities NEC | 1 | (1.2%) | 2 | (2.4%) | 3 | (1.8%) | 7 | (2.7%) | 7 | (2.7%) | 14 | (2.7%) |
| Dysgeusia | 1 | (1.2%) | 1 | (1.2%) | 2 | (1.2%) | 3 | (1.2%) | 3 | (1.2%) | 6 | (1.2%) |
| Hypoaesthesia | 1 | (1.2%) | 0 | | 1 | (0.6%) | 4 | (1.6%) | 2 | (0.8%) | 6 | (1.2%) |
| HLGT: Peripheral neuropathies | 1 | (1.2%) | 2 | (2.4%) | 3 | (1.8%) | 6 | (2.4%) | 13 | (5.1%) | 19 | (3.7%) |
| HLT: Chronic polyneuropathies | 1 | (1.2%) | 1 | (1.2%) | 2 | (1.2%) | 2 | (0.8%) | 4 | (1.6%) | 6 | (1.2%) |
| Diabetic neuropathy | 1 | (1.2%) | 1 | (1.2%) | 2 | (1.2%) | 2 | (0.8%) | 4 | (1.6%) | 6 | (1.2%) |
| HLT: Peripheral neuropathies NEC | 1 | (1.2%) | 1 | (1.2%) | 2 | (1.2%) | 0 | | 6 | (2.4%) | 6 | (1.2%) |
| Neuropathy peripheral | 0 | | 1 | (1.2%) | 1 | (0.6%) | 0 | | 5 | (2.0%) | 5 | (1.0%) |
| HLGT: Spinal cord and nerve root disorders | 1 | (1.2%) | 2 | (2.4%) | 3 | (1.8%) | 7 | (2.7%) | 5 | (2.0%) | 12 | (2.4%) |
| HLT: Lumbar spinal cord and nerve root | 1 | (1.2%) | 2 | (2.4%) | 3 | (1.8%) | 5 | (2.0%) | 5 | (2.0%) | 10 | (2.0%) |
| disorders | | | | | | | | | | | | |
| Sciatica | 1 | (1.2%) | 2 | (2.4%) | 3 | (1.8%) | 4 | (1.6%) | 5 | (2.0%) | 9 | (1.8%) |
| EYE DISORDERS | 2 | (2.4%) | 5 | (5.9%) | 7 | (4.1%) | 18 | (7.1%) | 11 | (4.3%) | 29 | (5.7%) |
| HLGT: Anterior eye structural change, deposit | 0 | | 0 | | 0 | | 4 | (1.6%) | 3 | (1.2%) | 7 | (1.4%) |
| and degeneration | | | | | | | | | | | | |
| HLT: Cataract conditions | 0 | | 0 | | 0 | | 4 | (1.6%) | 2 | (0.8%) | 6 | (1.2%) |
| Cataract | 0 | | 0 | | 0 | | 4 | (1.6%) | 2 | (0.8%) | 6 | (1.2%) |
| HLGT: Ocular infections, irritations and | 1 | (1.2%) | 1 | (1.2%) | 2 | (1.2%) | 3 | (1.2%) | 7 | (2.7%) | 10 | (2.0%) |
| inflammations | | | | | | | | | | | | |
| HLT: Conjunctival infections, irritations and | 1 | (1.2%) | 1 | (1.2%) | 2 | (1.2%) | 2 | (0.8%) | 4 | (1.6%) | 6 | (1.2%) |
| inflammations | | | | | | | | | | | | |
| Conjunctivitis | 1 | (1.2%) | 1 | (1.2%) | 2 | (1.2%) | 1 | (0.4%) | 3 | (1.2%) | 4 | (0.8%) |
| HLGT: Vision disorders | 1 | (1.2%) | 3 | (3.5%) | 4 | (2.4%) | 10 | (3.9%) | 0 | | 10 | (2.0%) |
| HLT: Visual disorders NEC | 1 | (1.2%) | 2 | (2.4%) | 3 | (1.8%) | 8 | (3.1%) | 0 | | 8 | (1.6%) |
| Vision blurred | 1 | (1.2%) | 2 | (2.4%) | 3 | (1.8%) | 5 | (2.0%) | 0 | | 5 | (1.0%) |
| EAR AND LABYRINTH DISORDERS | 2 | (2.4%) | 5 | (5.9%) | 7 | (4.1%) | 8 | (3.1%) | 9 | (3.5%) | 17 | (3.3%) |
| HLGT: Aural disorders NEC | 2 | (2.4%) | 1 | (1.2%) | 3 | (1.8%) | 3 | (1.2%) | 0 | | 3 | (0.6%) |
| HLT: Ear disorders NEC | 2 | (2.4%) | 1 | (1.2%) | 3 | (1.8%) | 3 | (1.2%) | 0 | | 3 | (0.6%) |
| Ear pain | 2 | (2.4%) | 1 | (1.2%) | 3 | (1.8%) | 1 | (0.4%) | 0 | | 1 | (0.2%) |
| HLGT: Inner ear and VIIIth cranial nerve | 1 | (1.2%) | 4 | (4.7%) | 5 | (2.9%) | 5 | (2.0%) | 9 | (3.5%) | 14 | (2.7%) |
| disorders | | | | | | | | | | | | |
| HLT: Inner ear signs and symptoms | 1 | (1.2%) | 4 | (4.7%) | 5 | (2.9%) | 5 | (2.0%) | 9 | (3.5%) | 14 | (2.7%) |
| Vertigo | 1 | (1.2%) | 2 | (2.4%) | 3 | (1.8%) | 3 | (1.2%) | 6 | (2.4%) | 9 | (1.8%) |
| CARDIAC DISORDERS | 2 | (2.4%) | 3 | (3.5%) | 5 | (2.9%) | 17 | (6.7%) | 21 | (8.2%) | 38 | (7.5%) |
| HLGT: Cardiac arrhythmias | 2 | (2.4%) | 2 | (2.4%) | 4 | (2.4%) | 8 | (3.1%) | 14 | (5.5%) | 22 | (4.3%) |
| HLT: Rate and rhythm disorders NEC | 1 | (1.2%) | 0 | | 1 | (0.6%) | 4 | (1.6%) | 5 | (2.0%) | 9 | (1.8%) |
| Tachycardia | 1 | (1.2%) | 0 | | 1 | (0.6%) | 4 | (1.6%) | 4 | (1.6%) | 8 | (1.6%) |
| HLGT: Cardiac disorder signs and symptoms | 0 | | 0 | | 0 | | 2 | (0.8%) | 4 | (1.6%) | 6 | (1.2%) |
| HLT: Cardiac signs and symptoms NEC | 0 | | 0 | | 0 | | 2 | (0.8%) | 3 | (1.2%) | 5 | (1.0%) |
| Palpitations | 0 | | 0 | | 0 | | 2 | (0.8%) | 3 | (1.2%) | 5 | (1.0%) |
| HLGT: Coronary artery disorders | 0 | | 1 | (1.2%) | 1 | (0.6%) | 8 | (3.1%) | 5 | (2.0%) | 13 | (2.5%) |
| HLT: Ischaemic coronary artery disorders | 0 | | 1 | (1.2%) | 1 | (0.6%) | 7 | (2.7%) | 5 | (2.0%) | 12 | (2.4%) |
| Angina pectoris | 0 | | 0 | | 0 | | 4 | (1.6%) | 3 | (1.2%) | 7 | (1.4%) |
| VASCULAR DISORDERS | 7 | (8.2%) | 13 | (15.3%) | 20 | (11.8%) | 26 | (10.2%) | 28 | (11.0%) | 54 | (10.6%) |
| HLGT: Decreased and nonspecific blood pressure | 1 | (1.2%) | 1 | (1.2%) | 2 | (1.2%) | 2 | (0.8%) | 3 | (1.2%) | 5 | (1.0%) |
| disorders and shock | | | | | | | | | | | | |
| HLT: Vascular hypotensive disorders | 1 | (1.2%) | 1 | (1.2%) | 2 | (1.2%) | 2 | (0.8%) | 3 | (1.2%) | 5 | (1.0%) |
| Hypotension | 1 | (1.2%) | 1 | (1.2%) | 2 | (1.2%) | 2 | (0.8%) | 2 | (0.8%) | 4 | (0.8%) |
| HLGT: Vascular disorders NEC | 2 | (2.4%) | 3 | (3.5%) | 5 | (2.9%) | 1 | (0.4%) | 5 | (2.0%) | 6 | (1.2%) |
| HLT: Peripheral vascular disorders NEC | 2 | (2.4%) | 2 | (2.4%) | 4 | (2.4%) | 1 | (0.4%) | 5 | (2.0%) | 6 | (1.2%) |
| Hot flush | 1 | (1.2%) | 2 | (2.4%) | 3 | (1.8%) | 1 | (0.4%) | 4 | (1.6%) | 5 | (1.0%) |
| HLGT: Vascular hypertensive disorders | 3 | (3.5%) | 7 | (8.2%) | 10 | (5.9%) | 21 | (8.2%) | 18 | (7.1%) | 39 | (7.6%) |
| HLT: Accelerated and malignant | 0 | | 1 | (1.2%) | 1 | (0.6%) | 5 | (2.0%) | 4 | (1.6%) | 9 | (1.8%) |
| hypertension | | | | | | | | | | | | |
| Hypertensive crisis | 0 | | 1 | (1.2%) | 1 | (0.6%) | 5 | (2.0%) | 4 | (1.6%) | 9 | (1.8%) |
| HLT: Vascular hypertensive disorders NEC | 3 | (3.5%) | 6 | (7.1%) | 9 | (5.3%) | 17 | (6.7%) | 15 | (5.9%) | 32 | (6.3%) |
| Hypertension | 3 | (3.5%) | 6 | (7.1%) | 9 | (5.3%) | 17 | (6.7%) | 15 | (5.9%) | 32 | (6.3%) |
| RESPIRATORY, THORACIC AND | 7 | (8.2%) | 13 | (15.3%) | 20 | (11.8%) | 30 | (11.8%) | 24 | (9.4%) | 54 | (10.6%) |
| MEDIASTINAL DISORDERS | | | | | | | | | | | | |
| HLGT: Bronchial disorders (excl neoplasms) | 2 | (2.4%) | 1 | (1.2%) | 3 | (1.8%) | 4 | (1.6%) | 1 | (0.4%) | 5 | (1.0%) |
| HLT: Bronchospasm and obstruction | 1 | (1.2%) | 1 | (1.2%) | 2 | (1.2%) | 4 | (1.6%) | 1 | (0.4%) | 5 | (1.0%) |
| Asthma | 0 | | 0 | | 0 | | 3 | (1.2%) | 0 | | 3 | (0.6%) |
| HLGT: Respiratory disorders NEC | 5 | (5.9%) | 8 | (9.4%) | 13 | (7.6%) | 23 | (9.0%) | 19 | (7.5%) | 42 | (8.2%) |
| HLT: Coughing and associated symptoms | 3 | (3.5%) | 6 | (7.1%) | 9 | (5.3%) | 12 | (4.7%) | 10 | (3.9%) | 22 | (4.3%) |
| Cough | 2 | (2.4%) | 5 | (5.9%) | 7 | (4.1%) | 10 | (3.9%) | 9 | (3.5%) | 19 | (3.7%) |
| HLT: Respiratory tract disorders NEC | 1 | (1.2%) | 1 | (1.2%) | 2 | (1.2%) | 0 | | 3 | (1.2%) | 3 | (0.6%) |
| Respiratory disorder | 1 | (1.2%) | 0 | | 1 | (0.6%) | 0 | | 3 | (1.2%) | 3 | (0.6%) |
| HLT: Upper respiratory tract signs and | 2 | (2.4%) | 2 | (2.4%) | 4 | (2.4%) | 11 | (4.3%) | 7 | (2.7%) | 18 | (3.5%) |
| symptoms | | | | | | | | | | | | |
| Dysphonia | 1 | (1.2%) | 1 | (1.2%) | 2 | (1.2%) | 4 | (1.6%) | 0 | | 4 | (0.8%) |
| Oropharyngeal pain | 1 | (1.2%) | 1 | (1.2%) | 2 | (1.2%) | 6 | (2.4%) | 6 | (2.4%) | 12 | (2.4%) |
| Rhinorrhoea | 0 | | 0 | | 0 | | 3 | (1.2%) | 0 | | 3 | (0.6%) |
| HLGT: Upper respiratory tract disorders (excl | 2 | (2.4%) | 5 | (5.9%) | 7 | (4.1%) | 4 | (1.6%) | 2 | (0.8%) | 6 | (1.2%) |
| infections) | | | | | | | | | | | | |
| HLT: Nasal congestion and inflammations | 1 | (1.2%) | 2 | (2.4%) | 3 | (1.8%) | 2 | (0.8%) | 0 | | 2 | (0.4%) |
| Nasal congestion | 1 | (1.2%) | 1 | (1.2%) | 2 | (1.2%) | 0 | | 0 | | 0 | |
| HLT: Nasal disorders NEC | 0 | | 3 | (3.5%) | 3 | (1.8%) | 2 | (0.8%) | 2 | (0.8%) | 4 | (0.8%) |
| Epistaxis | 0 | | 3 | (3.5%) | 3 | (1.8%) | 2 | (0.8%) | 1 | (0.4%) | 3 | (0.6%) |
| GASTROINTESTINAL DISORDERS | 26 | (30.6%) | 31 | (36.5%) | 57 | (33.5%) | 129 | (50.6%) | 122 | (47.8%) | 251 | (49.2%) |
| HLGT: Dental and gingival conditions | 3 | (3.5%) | 5 | (5.9%) | 8 | (4.7%) | 7 | (2.7%) | 13 | (5.1%) | 20 | (3.9%) |
| HLT: Dental pain and sensation disorders | 2 | (2.4%) | 3 | (3.5%) | 5 | (2.9%) | 4 | (1.6%) | 7 | (2.7%) | 11 | (2.2%) |
| Toothache | 2 | (2.4%) | 3 | (3.5%) | 5 | (2.9%) | 4 | (1.6%) | 7 | (2.7%) | 11 | (2.2%) |
| HLT: Gingival disorders NEC | 0 | | 2 | (2.4%) | 2 | (1.2%) | 1 | (0.4%) | 4 | (1.6%) | 5 | (1.0%) |
| Gingivitis | 0 | | 2 | (2.4%) | 2 | (1.2%) | 0 | | 4 | (1.6%) | 4 | (0.8%) |
| HLGT: Gastrointestinal conditions NEC | 0 | | 0 | | 0 | | 4 | (1.6%) | 1 | (0.4%) | 5 | (1.0%) |
| HLT: Gastrointestinal disorders NEC | 0 | | 0 | | 0 | | 4 | (1.6%) | 0 | | 4 | (0.8%) |
| Food poisoning | 0 | | 0 | | 0 | | 4 | (1.6%) | 0 | | 4 | (0.8%) |
| HLGT: Gastrointestinal inflammatory conditions | 2 | (2.4%) | 1 | (1.2%) | 3 | (1.8%) | 8 | (3.1%) | 10 | (3.9%) | 18 | (3.5%) |
| HLT: Gastritis (excl infective) | 1 | (1.2%) | 0 | | 1 | (0.6%) | 5 | (2.0%) | 9 | (3.5%) | 14 | (2.7%) |
| Gastritis | 1 | (1.2%) | 0 | | 1 | (0.6%) | 5 | (2.0%) | 9 | (3.5%) | 14 | (2.7%) |
| HLGT: Gastrointestinal motility and defaecation | 12 | (14.1%) | 11 | (12.9%) | 23 | (13.5%) | 47 | (18.4%) | 46 | (18.0%) | 93 | (18.2%) |
| conditions | | | | | | | | | | | | |
| HLT: Diarrhoea (excl infective) | 10 | (11.8%) | 10 | (11.8%) | 20 | (11.8%) | 39 | (15.3%) | 36 | (14.1%) | 75 | (14.7%) |
| Diarrhoea | 10 | (11.8%) | 10 | (11.8%) | 20 | (11.8%) | 39 | (15.3%) | 36 | (14.1%) | 75 | (14.7%) |
| HLT: Gastrointestinal atonic and | 3 | (3.5%) | 1 | (1.2%) | 4 | (2.4%) | 8 | (3.1%) | 14 | (5.5%) | 22 | (4.3%) |
| hypomotility disorders NEC | | | | | | | | | | | | |
| Constipation | 1 | (1.2%) | 1 | (1.2%) | 2 | (1.2%) | 6 | (2.4%) | 7 | (2.7%) | 13 | (2.5%) |
| Gastrooesophageal reflux disease | 2 | (2.4%) | 0 | | 2 | (1.2%) | 2 | (0.8%) | 7 | (2.7%) | 9 | (1.8%) |
| HLT: Gastrointestinal spastic and | 1 | (1.2%) | 0 | | 1 | (0.6%) | 3 | (1.2%) | 1 | (0.4%) | 4 | (0.8%) |
| hypermotility disorders | | | | | | | | | | | | |
| Irritable bowel syndrome | 1 | (1.2%) | 0 | | 1 | (0.6%) | 3 | (1.2%) | 1 | (0.4%) | 4 | (0.8%) |
| HLGT: Gastrointestinal signs and symptoms | 14 | (16.5%) | 20 | (23.5%) | 34 | (20.0%) | 93 | (36.5%) | 98 | (38.4%) | 191 | (37.5%) |
| HLT: Dyspeptic signs and symptoms | 1 | (1.2%) | 1 | (1.2%) | 2 | (1.2%) | 16 | (6.3%) | 14 | (5.5%) | 30 | (5.9%) |
| Dyspepsia | 1 | (1.2%) | 0 | | 1 | (0.6%) | 15 | (5.9%) | 13 | (5.1%) | 28 | (5.5%) |
| HLT: Flatulence, bloating and distension | 2 | (2.4%) | 3 | (3.5%) | 5 | (2.9%) | 10 | (3.9%) | 8 | (3.1%) | 18 | (3.5%) |
| Abdominal distension | 2 | (2.4%) | 1 | (1.2%) | 3 | (1.8%) | 6 | (2.4%) | 5 | (2.0%) | 11 | (2.2%) |
| Flatulence | 0 | | 2 | (2.4%) | 2 | (1.2%) | 5 | (2.0%) | 3 | (1.2%) | 8 | (1.6%) |
| HLT: Gastrointestinal and abdominal pains | 5 | (5.9%) | 7 | (8.2%) | 12 | (7.1%) | 23 | (9.0%) | 16 | (6.3%) | 39 | (7.6%) |
| (excl oral and throat) | | | | | | | | | | | | |
| Abdominal pain | 2 | (2.4%) | 2 | (2.4%) | 4 | (2.4%) | 14 | (5.5%) | 6 | (2.4%) | 20 | (3.9%) |
| Abdominal pain upper | 3 | (3.5%) | 6 | (7.1%) | 9 | (5.3%) | 11 | (4.3%) | 10 | (3.9%) | 21 | (4.1%) |
| HLT: Gastrointestinal signs and symptoms | 0 | | 0 | | 0 | | 3 | (1.2%) | 6 | (2.4%) | 9 | (1.8%) |
| NEC | | | | | | | | | | | | |
| Abdominal discomfort | 0 | | 0 | | 0 | | 3 | (1.2%) | 5 | (2.0%) | 8 | (1.6%) |
| HLT: Nausea and vomiting symptoms | 11 | (12.9%) | 12 | (14.1%) | 23 | (13.5%) | 76 | (29.8%) | 79 | (31.0%) | 155 | (30.4%) |
| Nausea | 7 | (8.2%) | 9 | (10.6%) | 16 | (9.4%) | 64 | (25.1%) | 63 | (24.7%) | 127 | (24.9%) |
| Vomiting | 6 | (7.1%) | 3 | (3.5%) | 9 | (5.3%) | 35 | (13.7%) | 40 | (15.7%) | 75 | (14.7%) |
| HLGT: Oral soft tissue conditions | 1 | (1.2%) | 2 | (2.4%) | 3 | (1.8%) | 6 | (2.4%) | 2 | (0.8%) | 8 | (1.6%) |
| HLT: Oral soft tissue pain and paraesthesia | 0 | | 1 | (1.2%) | 1 | (0.6%) | 4 | (1.6%) | 1 | (0.4%) | 5 | (1.0%) |
| Odynophagia | 0 | | 1 | (1.2%) | 1 | (0.6%) | 4 | (1.6%) | 1 | (0.4%) | 5 | (1.0%) |
| HEPATOBILIARY DISORDERS | 3 | (3.5%) | 8 | (9.4%) | 11 | (6.5%) | 6 | (2.4%) | 10 | (3.9%) | 16 | (3.1%) |
| HLGT: Gallbladder disorders | 0 | | 4 | (4.7%) | 4 | (2.4%) | 3 | (1.2%) | 3 | (1.2%) | 6 | (1.2%) |
| HLT: Cholecystitis and cholelithiasis | 0 | | 4 | (4.7%) | 4 | (2.4%) | 3 | (1.2%) | 2 | (0.8%) | 5 | (1.0%) |
| Cholelithiasis | 0 | | 3 | (3.5%) | 3 | (1.8%) | 3 | (1.2%) | 2 | (0.8%) | 5 | (1.0%) |
| HLGT: Hepatic and hepatobiliary disorders | 3 | (3.5%) | 4 | (4.7%) | 7 | (4.1%) | 2 | (0.8%) | 6 | (2.4%) | 8 | (1.6%) |
| HLT: Hepatocellular damage and hepatitis | 2 | (2.4%) | 3 | (3.5%) | 5 | (2.9%) | 2 | (0.8%) | 6 | (2.4%) | 8 | (1.6%) |
| NEC | | | | | | | | | | | | |
| Hepatic steatosis | 2 | (2.4%) | 3 | (3.5%) | 5 | (2.9%) | 2 | (0.8%) | 6 | (2.4%) | 8 | (1.6%) |
| SKIN AND SUBCUTANEOUS TISSUE | 5 | (5.9%) | 15 | (17.6%) | 20 | (11.8%) | 30 | (11.8%) | 31 | (12.2%) | 61 | (12.0%) |
| DISORDERS | | | | | | | | | | | | |
| HLGT: Angioedema and urticaria | 1 | (1.2%) | 1 | (1.2%) | 2 | (1.2%) | 2 | (0.8%) | 1 | (0.4%) | 3 | (0.6%) |
| HLT: Urticarias | 1 | (1.2%) | 1 | (1.2%) | 2 | (1.2%) | 1 | (0.4%) | 1 | (0.4%) | 2 | (0.4%) |
| Urticaria | 1 | (1.2%) | 1 | (1.2%) | 2 | (1.2%) | 1 | (0.4%) | 1 | (0.4%) | 2 | (0.4%) |
| HLGT: Epidermal and dermal conditions | 4 | (4.7%) | 7 | (8.2%) | 11 | (6.5%) | 16 | (6.3%) | 20 | (7.8%) | 36 | (7.1%) |
| HLT: Pruritus NEC | 0 | | 3 | (3.5%) | 3 | (1.8%) | 2 | (0.8%) | 4 | (1.6%) | 6 | (1.2%) |
| Pruritus | 0 | | 3 | (3.5%) | 3 | (1.8%) | 1 | (0.4%) | 3 | (1.2%) | 4 | (0.8%) |
| HLT: Rashes, eruptions and exanthems NEC | 0 | | 1 | (1.2%) | 1 | (0.6%) | 8 | (3.1%) | 8 | (3.1%) | 16 | (3.1%) |
| Rash | 0 | | 1 | (1.2%) | 1 | (0.6%) | 5 | (2.0%) | 7 | (2.7%) | 12 | (2.4%) |
| HLGT: Skin appendage conditions | 0 | | 6 | (7.1%) | 6 | (3.5%) | 12 | (4.7%) | 10 | (3.9%) | 22 | (4.3%) |
| HLT: Apocrine and eccrine gland disorders | 0 | | 3 | (3.5%) | 3 | (1.8%) | 8 | (3.1%) | 6 | (2.4%) | 14 | (2.7%) |
| Hyperhidrosis | 0 | | 2 | (2.4%) | 2 | (1.2%) | 6 | (2.4%) | 4 | (1.6%) | 10 | (2.0%) |
| MUSCULOSKELETAL AND CONNECTIVE | 19 | (22.4%) | 21 | (24.7%) | 40 | (23.5%) | 83 | (32.5%) | 76 | (29.8%) | 159 | (31.2%) |
| TISSUE DISORDERS | | | | | | | | | | | | |
| HLGT: Joint disorders | 6 | (7.1%) | 4 | (4.7%) | 10 | (5.9%) | 33 | (12.9%) | 21 | (8.2%) | 54 | (10.6%) |
| HLT: Joint related signs and symptoms | 4 | (4.7%) | 2 | (2.4%) | 6 | (3.5%) | 18 | (7.1%) | 11 | (4.3%) | 29 | (5.7%) |
| Arthralgia | 3 | (3.5%) | 2 | (2.4%) | 5 | (2.9%) | 18 | (7.1%) | 9 | (3.5%) | 27 | (5.3%) |
| HLT: Osteoarthropathies | 1 | (1.2%) | 2 | (2.4%) | 3 | (1.8%) | 14 | (5.5%) | 10 | (3.9%) | 24 | (4.7%) |
| Osteoarthritis | 1 | (1.2%) | 2 | (2.4%) | 3 | (1.8%) | 14 | (5.5%) | 10 | (3.9%) | 24 | (4.7%) |
| HLGT: Muscle disorders | 4 | (4.7%) | 4 | (4.7%) | 8 | (4.7%) | 13 | (5.1%) | 14 | (5.5%) | 27 | (5.3%) |
| HLT: Muscle pains | 3 | (3.5%) | 2 | (2.4%) | 5 | (2.9%) | 4 | (1.6%) | 9 | (3.5%) | 13 | (2.5%) |
| Myalgia | 3 | (3.5%) | 2 | (2.4%) | 5 | (2.9%) | 3 | (1.2%) | 9 | (3.5%) | 12 | (2.4%) |
| HLT: Muscle related signs and symptoms | 1 | (1.2%) | 2 | (2.4%) | 3 | (1.8%) | 6 | (2.4%) | 6 | (2.4%) | 12 | (2.4%) |
| NEC | | | | | | | | | | | | |
| Muscle spasms | 1 | (1.2%) | 2 | (2.4%) | 3 | (1.8%) | 6 | (2.4%) | 5 | (2.0%) | 11 | (2.2%) |
| HLGT: Musculoskeletal and connective tissue | 9 | (10.6%) | 10 | (11.8%) | 19 | (11.2%) | 52 | (20.4%) | 42 | (16.5%) | 94 | (18.4%) |
| disorders NEC | | | | | | | | | | | | |
| HLT: Musculoskeletal and connective tissue | 9 | (10.6%) | 10 | (11.8%) | 19 | (11.2%) | 49 | (19.2%) | 41 | (16.1%) | 90 | (17.6%) |
| pain and discomfort | | | | | | | | | | | | |
| Back pain | 6 | (7.1%) | 6 | (7.1%) | 12 | (7.1%) | 21 | (8.2%) | 21 | (8.2%) | 42 | (8.2%) |
| Musculoskeletal pain | 2 | (2.4%) | 3 | (3.5%) | 5 | (2.9%) | 9 | (3.5%) | 7 | (2.7%) | 16 | (3.1%) |
| Neck pain | 1 | (1.2%) | 0 | | 1 | (0.6%) | 6 | (2.4%) | 5 | (2.0%) | 11 | (2.2%) |
| Pain in extremity | 2 | (2.4%) | 1 | (1.2%) | 3 | (1.8%) | 18 | (7.1%) | 12 | (4.7%) | 30 | (5.9%) |
| HLGT: Tendon, ligament and cartilage disorders | 0 | | 2 | (2.4%) | 2 | (1.2%) | 10 | (3.9%) | 6 | (2.4%) | 16 | (3.1%) |
| HLT: Tendon disorders | 0 | | 2 | (2.4%) | 2 | (1.2%) | 9 | (3.5%) | 5 | (2.0%) | 14 | (2.7%) |
| Tendonitis | 0 | | 2 | (2.4%) | 2 | (1.2%) | 7 | (2.7%) | 2 | (0.8%) | 9 | (1.8%) |
| RENAL AND URINARY DISORDERS | 2 | (2.4%) | 7 | (8.2%) | 9 | (5.3%) | 13 | (5.1%) | 13 | (5.1%) | 26 | (5.1%) |
| HLGT: Urinary tract signs and symptoms | 2 | (2.4%) | 2 | (2.4%) | 4 | (2.4%) | 10 | (3.9%) | 9 | (3.5%) | 19 | (3.7%) |
| HLT: Urinary abnormalities | 1 | (1.2%) | 0 | | 1 | (0.6%) | 3 | (1.2%) | 4 | (1.6%) | 7 | (1.4%) |
| Microalbuminuria | 0 | | 0 | | 0 | | 0 | | 3 | (1.2%) | 3 | (0.6%) |
| HLT: Urinary tract signs and symptoms NEC | 1 | (1.2%) | 1 | (1.2%) | 2 | (1.2%) | 3 | (1.2%) | 3 | (1.2%) | 6 | (1.2%) |
| Renal colic | 0 | | 0 | | 0 | | 2 | (0.8%) | 3 | (1.2%) | 5 | (1.0%) |
| HLGT: Urolithiases | 0 | | 2 | (2.4%) | 2 | (1.2%) | 2 | (0.8%) | 2 | (0.8%) | 4 | (0.8%) |
| HLT: Renal lithiasis | 0 | | 2 | (2.4%) | 2 | (1.2%) | 1 | (0.4%) | 2 | (0.8%) | 3 | (0.6%) |
| Nephrolithiasis | 0 | | 2 | (2.4%) | 2 | (1.2%) | 1 | (0.4%) | 2 | (0.8%) | 3 | (0.6%) |
| REPRODUCTIVE SYSTEM AND BREAST | 5 | (5.9%) | 2 | (2.4%) | 7 | (4.1%) | 14 | (5.5%) | 7 | (2.7%) | 21 | (4.1%) |
| DISORDERS | | | | | | | | | | | | |
| HLGT: Menstrual cycle and uterine bleeding | 1 | (1.2%) | 0 | | 1 | (0.6%) | 5 | (2.0%) | 2 | (0.8%) | 7 | (1.4%) |
| disorders | | | | | | | | | | | | |
| HLT: Menstruation and uterine bleeding | 1 | (1.2%) | 0 | | 1 | (0.6%) | 5 | (2.0%) | 0 | | 5 | (1.0%) |
| NEC | | | | | | | | | | | | |
| Metrorrhagia | 0 | | 0 | | 0 | | 3 | (1.2%) | 0 | | 3 | (0.6%) |
| HLGT: Sexual function and fertility disorders | 2 | (2.4%) | 0 | | 2 | (1.2%) | 1 | (0.4%) | 1 | (0.4%) | 2 | (0.4%) |
| HLT: Erection and ejaculation conditions and | 2 | (2.4%) | 0 | | 2 | (1.2%) | 1 | (0.4%) | 1 | (0.4%) | 2 | (0.4%) |
| disorders | | | | | | | | | | | | |
| Erectile dysfunction | 2 | (2.4%) | 0 | | 2 | (1.2%) | 0 | | 1 | (0.4%) | 1 | (0.2%) |
| GENERAL DISORDERS AND | 8 | (9.4%) | 15 | (17.6%) | 23 | (13.5%) | 63 | (24.7%) | 56 | (22.0%) | 119 | (23.3%) |
| ADMINISTRATION SITE CONDITIONS | | | | | | | | | | | | |
| HLGT: Administration site reactions | 3 | (3.5%) | 1 | (1.2%) | 4 | (2.4%) | 18 | (7.1%) | 17 | (6.7%) | 35 | (6.9%) |
| HLT: Injection site reactions | 3 | (3.5%) | 1 | (1.2%) | 4 | (2.4%) | 17 | (6.7%) | 15 | (5.9%) | 32 | (6.3%) |
| Injection site haematoma | 1 | (1.2%) | 0 | | 1 | (0.6%) | 4 | (1.6%) | 3 | (1.2%) | 7 | (1.4%) |
| Injection site pain | 2 | (2.4%) | 1 | (1.2%) | 3 | (1.8%) | 6 | (2.4%) | 4 | (1.6%) | 10 | (2.0%) |
| Injection site reaction | 0 | | 0 | | 0 | | 6 | (2.4%) | 2 | (0.8%) | 8 | (1.6%) |
| HLGT: Body temperature conditions | 0 | | 4 | (4.7%) | 4 | (2.4%) | 3 | (1.2%) | 4 | (1.6%) | 7 | (1.4%) |
| HLT: Febrile disorders | 0 | | 4 | (4.7%) | 4 | (2.4%) | 3 | (1.2%) | 4 | (1.6%) | 7 | (1.4%) |
| Pyrexia | 0 | | 4 | (4.7%) | 4 | (2.4%) | 3 | (1.2%) | 4 | (1.6%) | 7 | (1.4%) |
| HLGT: General system disorders NEC | 6 | (7.1%) | 10 | (11.8%) | 16 | (9.4%) | 43 | (16.9%) | 40 | (15.7%) | 83 | (16.3%) |
| HLT: Asthenic conditions | 4 | (4.7%) | 5 | (5.9%) | 9 | (5.3%) | 28 | (11.0%) | 25 | (9.8%) | 53 | (10.4%) |
| Asthenia | 1 | (1.2%) | 2 | (2.4%) | 3 | (1.8%) | 11 | (4.3%) | 11 | (4.3%) | 22 | (4.3%) |
| Fatigue | 3 | (3.5%) | 2 | (2.4%) | 5 | (2.9%) | 14 | (5.5%) | 9 | (3.5%) | 23 | (4.5%) |
| Malaise | 0 | | 1 | (1.2%) | 1 | (0.6%) | 5 | (2.0%) | 6 | (2.4%) | 11 | (2.2%) |
| HLT: Feelings and sensations NEC | 0 | | 2 | (2.4%) | 2 | (1.2%) | 5 | (2.0%) | 6 | (2.4%) | 11 | (2.2%) |
| Chills | 0 | | 0 | | 0 | | 4 | (1.6%) | 3 | (1.2%) | 7 | (1.4%) |
| HLT: Oedema NEC | 1 | (1.2%) | 1 | (1.2%) | 2 | (1.2%) | 5 | (2.0%) | 8 | (3.1%) | 13 | (2.5%) |
| Oedema peripheral | 0 | | 1 | (1.2%) | 1 | (0.6%) | 4 | (1.6%) | 8 | (3.1%) | 12 | (2.4%) |
| HLT: Pain and discomfort NEC | 1 | (1.2%) | 1 | (1.2%) | 2 | (1.2%) | 10 | (3.9%) | 4 | (1.6%) | 14 | (2.7%) |
| Chest pain | 1 | (1.2%) | 0 | | 1 | (0.6%) | 3 | (1.2%) | 4 | (1.6%) | 7 | (1.4%) |
| Non-cardiac chest pain | 0 | | 1 | (1.2%) | 1 | (0.6%) | 3 | (1.2%) | 0 | | 3 | (0.6%) |
| INVESTIGATIONS | 6 | (7.1%) | 10 | (11.8%) | 16 | (9.4%) | 28 | (11.0%) | 29 | (11.4%) | 57 | (11.2%) |
| HLGT: Cardiac and vascular investigations (excl | 1 | (1.2%) | 0 | | 1 | (0.6%) | 4 | (1.6%) | 2 | (0.8%) | 6 | (1.2%) |
| enzyme tests) | | | | | | | | | | | | |
| HLT: Vascular tests NEC (incl blood | 0 | | 0 | | 0 | | 3 | (1.2%) | 2 | (0.8%) | 5 | (1.0%) |
| pressure) | | | | | | | | | | | | |
| Blood pressure increased | 0 | | 0 | | 0 | | 3 | (1.2%) | 1 | (0.4%) | 4 | (0.8%) |
| HLGT: Endocrine investigations (incl sex | 0 | | 3 | (3.5%) | 3 | (1.8%) | 5 | (2.0%) | 4 | (1.6%) | 9 | (1.8%) |
| hormones) | | | | | | | | | | | | |
| HLT: Gastrointestinal, pancreatic and APUD | 0 | | 3 | (3.5%) | 3 | (1.8%) | 5 | (2.0%) | 4 | (1.6%) | 9 | (1.8%) |
| hormone analyses | | | | | | | | | | | | |
| Blood calcitonin increased | 0 | | 3 | (3.5%) | 3 | (1.8%) | 5 | (2.0%) | 4 | (1.6%) | 9 | (1.8%) |
| HLGT: Gastrointestinal investigations | 1 | (1.2%) | 4 | (4.7%) | 5 | (2.9%) | 7 | (2.7%) | 11 | (4.3%) | 18 | (3.5%) |
| HLT: Digestive enzymes | 1 | (1.2%) | 4 | (4.7%) | 5 | (2.9%) | 7 | (2.7%) | 11 | (4.3%) | 18 | (3.5%) |
| Blood amylase increased | 1 | (1.2%) | 2 | (2.4%) | 3 | (1.8%) | 3 | (1.2%) | 5 | (2.0%) | 8 | (1.6%) |
| Lipase increased | 1 | (1.2%) | 3 | (3.5%) | 4 | (2.4%) | 6 | (2.4%) | 9 | (3.5%) | 15 | (2.9%) |
| HLGT: Hepatobiliary investigations | 1 | (1.2%) | 1 | (1.2%) | 2 | (1.2%) | 2 | (0.8%) | 10 | (3.9%) | 12 | (2.4%) |
| HLT: Liver function analyses | 1 | (1.2%) | 1 | (1.2%) | 2 | (1.2%) | 2 | (0.8%) | 10 | (3.9%) | 12 | (2.4%) |
| Gamma-glutamyltransferase increased | 1 | (1.2%) | 1 | (1.2%) | 2 | (1.2%) | 1 | (0.4%) | 3 | (1.2%) | 4 | (0.8%) |
| Hepatic enzyme increased | 0 | | 0 | | 0 | | 1 | (0.4%) | 5 | (2.0%) | 6 | (1.2%) |
| HLGT: Lipid analyses | 2 | (2.4%) | 1 | (1.2%) | 3 | (1.8%) | 1 | (0.4%) | 3 | (1.2%) | 4 | (0.8%) |
| HLT: Cholesterol analyses | 1 | (1.2%) | 1 | (1.2%) | 2 | (1.2%) | 1 | (0.4%) | 0 | | 1 | (0.2%) |
| Blood cholesterol increased | 1 | (1.2%) | 1 | (1.2%) | 2 | (1.2%) | 1 | (0.4%) | 0 | | 1 | (0.2%) |
| HLT: Triglyceride analyses | 1 | (1.2%) | 1 | (1.2%) | 2 | (1.2%) | 0 | | 3 | (1.2%) | 3 | (0.6%) |
| Blood triglycerides increased | 1 | (1.2%) | 1 | (1.2%) | 2 | (1.2%) | 0 | | 3 | (1.2%) | 3 | (0.6%) |
| HLGT: Renal and urinary tract investigations and | 0 | | 0 | | 0 | | 3 | (1.2%) | 0 | | 3 | (0.6%) |
| urinalyses | | | | | | | | | | | | |
| HLT: Renal function analyses | 0 | | 0 | | 0 | | 3 | (1.2%) | 0 | | 3 | (0.6%) |
| Blood creatinine increased | 0 | | 0 | | 0 | | 3 | (1.2%) | 0 | | 3 | (0.6%) |
| INJURY, POISONING AND PROCEDURAL | 12 | (14.1%) | 13 | (15.3%) | 25 | (14.7%) | 30 | (11.8%) | 26 | (10.2%) | 56 | (11.0%) |
| COMPLICATIONS | | | | | | | | | | | | |
| HLGT: Bone and joint injuries | 3 | (3.5%) | 5 | (5.9%) | 8 | (4.7%) | 11 | (4.3%) | 10 | (3.9%) | 21 | (4.1%) |
| HLT: Limb injuries NEC (incl traumatic | 3 | (3.5%) | 1 | (1.2%) | 4 | (2.4%) | 7 | (2.7%) | 4 | (1.6%) | 11 | (2.2%) |
| amputation) | | | | | | | | | | | | |
| Limb injury | 1 | (1.2%) | 0 | | 1 | (0.6%) | 3 | (1.2%) | 4 | (1.6%) | 7 | (1.4%) |
| HLT: Thoracic cage fractures and | 0 | | 2 | (2.4%) | 2 | (1.2%) | 1 | (0.4%) | 2 | (0.8%) | 3 | (0.6%) |
| dislocations | | | | | | | | | | | | |
| Rib fracture | 0 | | 2 | (2.4%) | 2 | (1.2%) | 1 | (0.4%) | 2 | (0.8%) | 3 | (0.6%) |
| HLGT: Injuries NEC | 9 | (10.6%) | 11 | (12.9%) | 20 | (11.8%) | 19 | (7.5%) | 19 | (7.5%) | 38 | (7.5%) |
| HLT: Non-site specific injuries NEC | 5 | (5.9%) | 5 | (5.9%) | 10 | (5.9%) | 8 | (3.1%) | 8 | (3.1%) | 16 | (3.1%) |
| Animal scratch | 0 | | 2 | (2.4%) | 2 | (1.2%) | 0 | | 0 | | 0 | |
| Fall | 1 | (1.2%) | 3 | (3.5%) | 4 | (2.4%) | 5 | (2.0%) | 5 | (2.0%) | 10 | (2.0%) |
| HLT: Skin injuries NEC | 5 | (5.9%) | 5 | (5.9%) | 10 | (5.9%) | 5 | (2.0%) | 8 | (3.1%) | 13 | (2.5%) |
| Contusion | 4 | (4.7%) | 4 | (4.7%) | 8 | (4.7%) | 3 | (1.2%) | 6 | (2.4%) | 9 | (1.8%) |
| SURGICAL AND MEDICAL PROCEDURES | 1 | (1.2%) | 2 | (2.4%) | 3 | (1.8%) | 9 | (3.5%) | 4 | (1.6%) | 13 | (2.5%) |
| HLGT: Head and neck therapeutic procedures | 1 | (1.2%) | 1 | (1.2%) | 2 | (1.2%) | 5 | (2.0%) | 2 | (0.8%) | 7 | (1.4%) |
| HLT: Dental and gingival therapeutic | 1 | (1.2%) | 1 | (1.2%) | 2 | (1.2%) | 5 | (2.0%) | 2 | (0.8%) | 7 | (1.4%) |
| procedures | | | | | | | | | | | | |
| Tooth extraction | 1 | (1.2%) | 1 | (1.2%) | 2 | (1.2%) | 5 | (2.0%) | 1 | (0.4%) | 6 | (1.2%) |

TEAE: Treatment emergent adverse event,
SOC: System Organ Class,
HLGT: High Level Group Term,
HLT: High Level Term,
PT: Preferred Term.
On-treatment period of the whole study = the time from the first dose of double-blind study medication up to 3 days after the last dose administration.
MedDRA version: 13.1.
n (%) = number and percentage of patients with at least one TEAE.
Note:
Table sorted by SOC internationally agreed order and HLGT, HLT, PT alphabetic order.
Only SOC with at least one PT ≧1% (i.e. common TEAE) in the placebo combined group or any lixisenatide morning or evening injection arm are presented.

1. A pharmaceutical combination for use in the treatment of a diabetes type 2 patient, said combination comprising
(a) desPro36Exendin-4(1-39)-Lys6—NH2 or/and a pharmaceutically acceptable salt thereof, and
(b) metformin or/and a pharmaceutically acceptable salt thereof,
wherein compound (a) is administered once daily before an evening meal.

2-10. (canceled)

11. The pharmaceutical combination of claim 1, wherein the desPro³⁶Exendin-4(1-39)-Lys₆-NH₂ or/and the pharmaceutically acceptable salt thereof is prepared for parenteral administration.

12. The pharmaceutical combination according to claim 1, wherein the desPro³⁶Exendin-4(1-39)-Lys₆-NH₂ or/and the pharmaceutically acceptable salt thereof is prepared for administration in a daily dose selected from the range of 10 μg to 20 μg.

13. The pharmaceutical combination of claim 1, wherein the metformin or/and the pharmaceutically acceptable salt thereof is prepared for oral administration.

14. A method of treating a diabetes type 2 patient, said method comprising administering desPro³⁶Exendin-4(1-39)-Lys₆-NH₂ or/and a pharmaceutically acceptable salt thereof, in combination with metformin to said patient in need thereof, comprising administering the pharmaceutical combination of claim 1, wherein compound (a) is administered once daily before an evening meal.

15. The method of claim 14, wherein said patient is obese.

16. The method according to claim 14, wherein said patient has a body mass index of at least 30 kg/m².

17. The method of claim 14, wherein said patient is an adult subject.

18. The method of claim 14, wherein said patient does not receive an antidiabetic treatment.

19. The method of claim 14, wherein diabetes mellitus type 2 has been diagnosed in said patient at least 1 year or at least 2 years before onset of therapy.

20. The method of claim 14 wherein said patient has a HbA_{1c} value of about 7 to about 10%.

21. The method of claim 14, wherein said patient has a fasting plasma glucose concentration of at least 8 mmol/L.

22. The method of claim 14, wherein said patient has a 2 hour postprandial plasma glucose concentration of at least 10, 12 or 14 mmol/L.

23. The method of claim 22, wherein said patient has a glucose excursion of at least 2, 3, 4 or 5 mmol/L, wherein the glucose excursion is the difference of the 2 hour postprandial plasma glucose concentration and plasma glucose concentration 30 minutes prior to a meal test.
